(19) United States
(12) Patent Application Publication
(10) Pub. No.: US 2026/0074057 A1
(43) Pub. Date: Mar. 12, 2026

(54) DENSITY-BASED IMMUNOPHENOTYPING

(71) Applicants: Genentech, Inc., South San Francisco, CA (US); Roche Sequencing Solutions, Inc., Pleasanton, CA (US)

(72) Inventors: Hauke KOLSTER, Fresno, CA (US); Cleopatra KOZLOWSKI, Woodside, CA (US); Daniel Lee RUDERMAN, San Francisco, CA (US); Patrick Joseph LEO, San Francisco, CA (US)

(21) Appl. No.: 19/391,554

(22) Filed: Nov. 17, 2025

Related U.S. Application Data
(63) Continuation of application No. PCT/US2024/030048, filed on May 17, 2024.
(60) Provisional application No. 63/503,144, filed on May 18, 2023.

(51) Int. Cl. G16H 30/40; G06T 7/00; G16H 20/17

(52) U.S. Cl. CPC G16H 30/40; G06T 7/0014; G16H 20/17; G06T 2207/10056; G06T 2207/20021; G06T 2207/20112; G06T 2207/30024; G06T 2207/30242

(57) ABSTRACT

Described herein are methods, systems, and programming for determining a tumor immunophenotype of an image of a tumor. Some embodiments include dividing an image into tiles depicting tumor epithelium and/or tumor stroma. For each tile, an epithelium-immune cell density and a stroma-immune cell density may be calculated based on a number of immune cells identified in the tumor epithelium and the tumor stroma, respectively. Based on the epithelium-immune cell density and the stroma-immune cell density, an inflammation type of the type may be determined, and a tumor immunophenotype may be determined based on each tile's inflammation type.

### CROSS-REFERENCE TO RELATED APPLICATION

This application is a continuation of International Application No. PCT/US2024/030048, filed on May 17, 2024, which claims the benefit of U.S. Provisional Application 63/503,144 filed on May 18, 2023, the entire content of which is incorporated herein by reference for all purposes.

### TECHNICAL FIELD

This application relates generally to processing digital pathology images of tumor lesions, and more particularly to computational-based methods for detecting a tumor lesion and determining an immunophenotype of the tumor lesion.

### BACKGROUND

One form of cancer immunotherapy involves checkpoint inhibitors (CIs), such as anti-PD-1/PD-L1 antibodies (e.g., atezolizumab, nivolumab, or docetaxel). However, immuno-oncology (IO) therapeutics, such as CIs, may have limited effect, which has triggered efforts to identify predictive biomarkers for IO therapies. To identify patients most likely to respond to such CIs, it may be useful to develop low-cost, simple, and reproducible biomarkers. One such biomarker is the PD-1/PD-L1 interaction. Immunohistochemistry (IHC) assays for the PD-1/PD-L1 molecule have achieved companion diagnostic status and are commonly used to identify patients likely to respond to CI therapeutics targeting the PD-1/PD-L1 axis, including CI therapeutics targeting non-small cell lung cancer. However, studies have demonstrated that a fraction of PD-L1 positive patients do not respond to CI-based therapy and a fraction of PD-L1 negative patients do respond, suggesting that the currently used biomarkers for IO therapies may be imperfect predictors of patient outcome.

The success of IO therapeutics relies on generating/facilitating anti-tumor immunity in the tumor microenvironment (TME). The TME may represent the spatial structure of tissue components and their microenvironment interactions. The complexity and plasticity of the TME poses a challenge to identify a single parameter with sufficient predictive power. Infiltration of the tumor bed by various cellular components of the immune system has been shown to carry prognostic value in solid tumor types.

The predictive/prognostic power of the density and spatial distribution of tumor-infiltrating lymphocytes (TILs) has been shown to be correlated with prognosis and/or treatment response. Evaluation of these characteristics may ignore local heterogeneity or the dynamics that lead up to the distribution of the TILs. Further, the method of TIL evaluation focuses on the stromal compartment of tumors and may not consider intra-epithelial immune cells. Yet, spatial localizations such as the local heterogeneity or the dynamics of the immune cells, in particular cytotoxic CD8+ T cells, may be an important factor in predicting patient response to immunotherapy. Evaluation of the pattern and density of immune infiltrates is, in most cases, based on visual inspection of a stained tissue section by a pathologist. This form of manual analysis is labor-intensive, subjective, error-prone, and associated with poor inter- and intra-observer concordance. It may be useful to provide one or more computational-based techniques for the automation of the evaluation of the pattern and density of immune infiltrates.

### SUMMARY

Some embodiments of the present disclosure include a method for determining an immunophenotype of a tumor using a computing system. The method may include receiving an image of a tumor and dividing the image into a plurality of tiles each depicting at least one of tumor epithelium or tumor stroma. For each of the plurality of tiles: an epithelium-immune cell density of the tile may be calculated based on a number of immune cells identified in the tumor epithelium, a stroma-immune cell density of the tile may be calculated based on a number of immune cells identified in the tumor stroma, and an inflammation type of the tile as being a first inflammation type or a second inflammation type may be determined based on the stroma-immune cell density and the epithelium-immune cell density. The method may further include determining a tumor immunophenotype for the image based on the inflammation type of the plurality of tiles.

Some embodiments of the present disclosure include a method for determining an immunophenotype of a tumor using a computing system. The method may include receiving an image of a tumor. Based on the image, an epithelium-stroma interface separating tumor epithelium and tumor stroma may be identified using one or more machine learning models. An epithelium-stroma interface immune cell density may be determined based on the image, where the epithelium-stroma immune cell density represents a number of immune cells within a threshold distance of the epithelium-stroma interface. An immune cell infiltration probability of immune cells in the tumor stroma infiltrating the tumor epithelium may be determined and determining a tumor immunophenotype of the image may be determined based on the epithelium-stroma interface immune cell density and the immune cell infiltration probability.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed can be resorted to by those skilled in the art, and that such modifications and variations are considered to be within the scope of this invention as defined by the appended claims.

According to some embodiments, an exemplary method for determining an immunophenotype of a tumor using a computing system can comprise: receiving an image of a tumor; dividing the image into a plurality of tiles each depicting at least one of tumor epithelium or tumor stroma; for each of the plurality of tiles: calculating an epithelium-immune cell density of the tile based on a number of immune cells identified in the tumor epithelium or calculating a stroma-immune cell density of the tile based on a number of immune cells identified in the tumor stroma; and determining based on the stroma-immune cell density and/or the epithelium-immune cell density, an inflammation type of the tile as being a first inflammation type or a second inflammation type; and determining a tumor immunophenotype for the image based on the inflammation type of the plurality of tiles.

In some embodiments, the tumor immunophenotype comprises: desert based on a number of tiles of the plurality of tiles of the first inflammation type being less than a first threshold and a number of tiles of the plurality of tiles of the second inflammation type being less than a second threshold; excluded based on the number of tiles of the plurality of tiles of the first inflammation type being greater than or equal to the first threshold and the number of tiles of the plurality of tiles of the second inflammation type being less than the second threshold; or inflamed based on the number of tiles of the plurality of tiles of the first inflammation type being greater than or equal to the first threshold and the number of tiles of the plurality of tiles of the second inflammation type being greater than or equal to the second threshold.

In some embodiments, the inflammation type comprises: the first inflammation type based on (i) a first stroma criterion for the stroma-immune cell density being met and (ii) a second stroma criterion for the epithelium-immune cell density being met; or the second inflammation type based on (iii) a first epithelium criterion for the stroma-immune cell density being met and (iv) a second epithelium criterion for the epithelium-immune cell density being met.

In some embodiments, the first stroma criterion for the stroma-immune cell density being met comprises the stroma-immune cell density being greater than or equal to a stroma-immune cell density threshold; the second stroma criterion for the epithelium-immune cell density being met comprises the epithelium-immune cell density being less than or equal to an epithelium-immune cell density threshold; the first epithelium criterion for the stroma-immune cell density being met comprises the stroma-immune cell density being less than the stroma-immune cell density threshold; and the second epithelium criterion for the epithelium-immune cell density being met comprises the epithelium-immune cell density being less than the epithelium-immune cell density threshold. In some embodiments, a first stroma criterion may comprise the stroma-immune cell density being less than or equal to a stroma-immune cell density threshold; a second stroma criterion may comprise an epithelium-immune cell density being greater than or equal to an epithelium-immune cell density threshold; a first epithelium criterion may include a stroma-immune cell density being greater than the stroma-immune cell density threshold, and a second epithelium criterion may include an epithelium-immune cell density being greater than the epithelium-immune cell density threshold.

In some embodiments, the stroma-immune cell density threshold and the epithelium-immune cell density threshold are based on a number of immune cells at an epithelium-stroma interface. In some embodiments, the stroma-immune cell density threshold and the epithelium-immune cell density threshold are based on a number of immune cells at an epithelium-stroma interface divided by a total number of tiles of the plurality of tiles.

In some embodiments, the stroma-immune cell density threshold is based on a distribution of immune cells in the tumor stroma and the epithelium-immune cell density threshold is based on a distribution of immune cells in the tumor epithelium, wherein the distribution of immune cells in the tumor stroma and the distribution of immune cells in the tumor epithelium is based on a plurality of distance measurements. In some embodiments, the method for determining an immunophenotype of a tumor further comprises: determining the plurality of distance measurements, comprising: performing a color deconvolution to generate a color channel highlighting cell nuclei; identifying based on the color channel, a plurality of immune cell nuclei; and calculating the plurality of distance measurements each representing a distance from one of the plurality of immune cell nuclei to an epithelium-stroma interface.

In some embodiments, the method for determining an immunophenotype of a tumor further comprises: performing a color deconvolution to generate a plurality of color channels from the image, the plurality of color channels including at least a first color channel and a second color channel, wherein the first color channel highlights immune cells and the second color channel distinguishes the tumor epithelium from the tumor stroma.

In some embodiments, the method for determining an immunophenotype of a tumor further comprises: determining a correction factor based on a number of immune cells at an epithelium-stroma interface; and modifying based on the correction factor, at least one of the calculated stroma-immune cell density or the calculated epithelium-immune cell density of at least some of the plurality of tiles.

In some embodiments, at least some of the plurality of tiles are overlapping. In some embodiments, at least one of the plurality of tiles contains a unique portion of the image. In some embodiments, at least one of the plurality of tiles comprises a random or pseudo-random subset of the plurality of tiles of the image. In some embodiments, the image comprises the tumor stained with one or more stains. The one or more stains can comprise at least one of: a pan-cytokeratin (panCK) stain used for highlighting the tumor epithelium; a cluster of differentiation 8 (CD8) stain used for highlighting immune cells; or a hematoxylin stain used for highlighting one or more of: cell nuclei, an extracellular matrix, or cell cytoplasm.

In some embodiments, the method for determining an immunophenotype of a tumor further comprises: identifying a boundary of the tumor in a digital pathology image; and extracting based on the boundary, the image of the tumor from the digital pathology image.

In some embodiments, identifying the boundary comprises: providing the digital pathology image to a computer vision model trained to detect the boundary of the tumor; and receiving an indication of the boundary from the computer vision model.

In some embodiments, the method for determining an immunophenotype of a tumor further comprises: selecting based on the tumor immunophenotype, an immunotherapy for a patient. In some embodiments, the method for determining an immunophenotype of a tumor further comprises: identifying artifacts in the image; and removing the artifacts from the image.

According to some embodiments, an exemplary system for determining an immunophenotype of a tumor can comprise: a computing system comprising one or more non-transitory computer-readable storage media storing computer program instructions; and one or more processors coupled to the one or more non-transitory computer-readable storage media, the one or more processors being configured to execute the computer program instructions to: receive an image of a tumor region; divide the image into a plurality of tiles each depicting at least one of tumor epithelium or tumor stroma; for each of the plurality of tiles; calculate an epithelium-immune cell density of the tile based on a number of immune cells identified in the tumor epithelium or calculate a stroma-immune cell density of the tile based on a number of immune cells identified in the tumor stroma; and determine, based on the stroma-immune cell density and/or the epithelium-immune cell density, an inflammation type of the tile as being a first inflammation type or a second inflammation type; and determine a tumor immunophenotype for the image based on the inflammation type of the plurality of tiles.

According to some embodiments, an exemplary non-transitory computer-readable medium can comprise computer program instructions that, when executed by one or more processors of a computing system, effectuate operations comprising: receiving an image of a tumor region; dividing the image into a plurality of tiles each depicting at least one of tumor epithelium or tumor stroma; for each of the plurality of tiles: calculating an epithelium-immune cell density of the tile based on a number of immune cells identified in the tumor epithelium or calculating a stroma-immune cell density of the tile based on a number of immune cells identified in the tumor stroma; and determining, based on the stroma-immune cell density and/or the epithelium-immune cell density, an inflammation type of the tile as being a first inflammation type or a second inflammation type; and determine a tumor immunophenotype for the image based on the inflammation type of the plurality of tiles.

According to some embodiments, an exemplary method for determining an immunophenotype of a tumor using a computing system can comprise: receiving an image of a tumor; identifying, based on the image, an epithelium-stroma interface separating tumor epithelium and tumor stroma using one or more machine learning models; determining an epithelium-stroma interface immune cell density based on the image, wherein the epithelium-stroma immune cell density represents a number of immune cells within a threshold distance of the epithelium-stroma interface; determining an immune cell infiltration probability of immune cells in the tumor stroma infiltrating the tumor epithelium; and determining a tumor immunophenotype of the image based on the epithelium-stroma interface immune cell density and the immune cell infiltration probability.

In some embodiments, identifying the epithelium-stroma interface comprises: separating the image into a plurality of color channels, wherein a first color channel of the plurality of color channels highlighting the immune cells and a second color channel distinguishing the tumor epithelium from the tumor stroma.

In some embodiments, identifying the epithelium-stroma interface comprises: identifying a boundary of the tumor in a digital pathology image the second color channel distinguishing the tumor epithelium and the tumor stroma, wherein the boundary comprises the epithelium-stroma interface; and extracting the image of the tumor from the digital pathology image based on the boundary.

In some embodiments, the one or more machine learning models comprise a computer vision model, the method further comprises: providing the image to the computer vision model trained to identify pixels highlighting the tumor epithelium and pixels highlighting the tumor stroma; and receiving an indication of the epithelium-stroma interface from the computer vision model.

In some embodiments, the image of the tumor is stained with one or more stains, wherein the one or more stains comprise at least one of: a pan-cytokeratin (panCK) stain used for highlighting the tumor epithelium; a cluster of differentiation 8 (CD8) stain used for highlighting immune cells; or a hematoxylin stain used for highlighting one or more of: cell nuclei, an extracellular matrix, or cell cytoplasm.

In some embodiments, determining the epithelium-stroma interface immune cell density comprises: separating the image into a plurality of color channels, wherein the plurality of color channels comprises a first color channel of the plurality of color channels highlighting the immune cells and at least a second color channel of the plurality of color channels distinguishing the tumor epithelium from the tumor stroma; and determining, based on the plurality of color channels, the number of immune cells within the threshold distance of the epithelium-stroma interface. In some embodiments, the threshold distance of the epithelium-stroma interface defines: a first distance from the epithelium-stroma interface into the tumor stroma; and a second distance from the epithelium-stroma interface into the tumor epithelium, and wherein the number of immune cells within the threshold distance comprises immune cells located within the first distance from the epithelium-stroma interface and immune cells located within the second distance from the epithelium-stroma interface. In some embodiments, at least one of the first distance or the second distance comprises 1 micron or less from the epithelium-stroma interface, 2 microns or less from the epithelium-stroma interface, 5 microns or less from the epithelium-stroma interface, 10 microns or less of the epithelium-stroma interface, or 20 microns or less from the epithelium-stroma interface.

In some embodiments, determining the immune cell infiltration probability comprises: computing a ratio of a number of immune cells depicted in the image that are located within the tumor stroma and a number of immune cells depicted in the image that are located within the tumor epithelium, wherein the immune cell infiltration probability is based on the ratio.

In some embodiments, the method for determining an immunophenotype of a tumor further comprises: accessing tumor immunophenotype classification data representing a tumor immunophenotype classification of each of a plurality of images of tumors based on an epithelium-stroma interface immune cell density and an immune cell infiltration probability of each of the plurality of images, wherein determining the tumor immunophenotype of the image comprises: classifying the image of the tumor into one of a set of tumor immunophenotypes based on the tumor immunophenotype classification data, the epithelium-stroma interface immune cell density of the image, and the immune cell infiltration probability of the image. In some embodiments, a trained classifier is used for classifying the image.

In some embodiments, the method for determining an immunophenotype of a tumor further comprises: for each of the plurality of images: identifying an epithelium-stroma interface; determining an epithelium-stroma interface immune cell density; determining an immune cell infiltration probability; and determining a tumor immunophenotype of the respective image based on the epithelium-stroma interface immune cell density of the respective image and the immune cell infiltration probability of the respective image. In some embodiments, the epithelium-stroma interface immune cell density of each of the plurality of images is determined using the one or more machine learning models.

In some embodiments, the set of tumor immunophenotypes comprises a first tumor immunophenotype and a second tumor immunophenotype. In some embodiments, the method for determining an immunophenotype of a tumor further comprises: computing a median epithelium-stroma interface immune cell density based the epithelium-stroma interface immune cell density of each of the plurality of images, wherein the image of the tumor is classified into one of the set of tumor immunophenotypes based on the median epithelium-stroma interface immune cell density and the immune cell infiltration probability. In some embodiments, the image is classified into one of the set of tumor immunophenotypes based on the median epithelium-stroma interface immune cell density. In some embodiments, the median epithelium-stroma interface immune cell density is less than 40 immune cells/mm², less than 60 immune cells/mm², or less than 100 immune cells/mm². In some embodiments, the median epithelium-stroma interface immune cell density is approximately 56 immune cells/mm².

In some embodiments, the method for determining an immunophenotype of a tumor further comprises: the first tumor immunophenotype is based on the epithelium-stroma interface immune cell density being less than the median epithelium-stroma interface immune cell density; and the second tumor immunophenotype is based on the epithelium-stroma interface immune cell density being greater than or equal to the median epithelium-stroma interface immune cell density. In some embodiments, the method for determining an immunophenotype of a tumor further comprises: the first tumor immunophenotype is based on the epithelium-stroma interface immune cell density being greater than or equal to the median epithelium-stroma interface immune cell density; and the second tumor immunophenotype is based on the epithelium-stroma interface immune cell density being less than or equal to the median epithelium-stroma interface immune cell density.

a

In some embodiments, the set of tumor immunophenotypes comprises: desert based on the epithelium-stroma interface immune cell density and the immune cell infiltration probability satisfying desert immunophenotype classification criteria; excluded based on the epithelium-stroma interface immune cell density and the immune cell infiltration probability satisfying excluded immunophenotype classification criteria; or inflamed based on the epithelium-stroma interface immune cell density and the immune cell infiltration probability satisfying inflamed immunophenotype classification criteria. In some embodiments, the desert immunophenotype classification criteria being satisfied comprises: the epithelium-stroma interface immune cell density being within a first threshold range of epithelium-stroma interface immune cell densities; and the immune cell infiltration probability being within a first threshold range of immune cell infiltration probabilities. In some embodiments, the excluded immunophenotype classification criteria being satisfied comprises: the epithelium-stroma interface immune cell density being within a second threshold range of epithelium-stroma interface immune cell densities; and the immune cell infiltration probability being within a second threshold range of immune cell infiltration probabilities. In some embodiments, the inflamed immunophenotype classification criteria being satisfied comprises: the epithelium-stroma interface immune cell density being within a third threshold range of epithelium-stroma interface immune cell densities; and the immune cell infiltration probability being within a third threshold range of immune cell infiltration probabilities.

In some embodiments, the method for determining an immunophenotype of a tumor further comprises: selecting an immunotherapy for a patient based on the tumor immunophenotype.

According to some embodiments, an exemplary system for determining an immunophenotype of a tumor can comprise: a computing system comprising one or more non-transitory computer-readable storage media storing computer program instructions; and one or more processors coupled to the one or more non-transitory computer-readable storage media, the one or more processors being configured to execute the computer program instructions to: receive an image of a tumor; identify, based on the image, an epithelium-stroma interface separating tumor epithelium and tumor stroma using one or more machine learning models; determine an epithelium-stroma interface immune cell density based on the image, wherein the epithelium-stroma interface immune cell density represents a number of immune cells within a threshold distance of the epithelium-stroma interface; determine an immune cell infiltration probability of immune cells in the tumor stroma infiltrating the tumor epithelium; and determine a tumor immunophenotype of the image based on the epithelium-stroma interface immune cell density and the immune cell infiltration probability.

According to some embodiments, an exemplary non-transitory computer-readable medium can comprise computer program instructions that, when executed by one or more processors of a computing system, effectuate operations comprising: receiving an image of a tumor; identifying, based on the image, an epithelium-stroma interface separating tumor epithelium and tumor stroma using one or more machine learning models; determining an epithelium-stroma interface immune cell density based on the image, wherein the epithelium-stroma interface immune cell density represents a number of immune cells within a threshold distance of the epithelium-stroma interface; determining an immune cell infiltration probability of immune cells in the tumor stroma infiltrating the tumor epithelium; and determining a tumor immunophenotype of the image based on the epithelium-stroma interface immune cell density and the immune cell infiltration probability.

According to some embodiments, an exemplary method for predicting a response to an anti-PD-L1 treatment by a patient can comprise: receiving an image of a tumor of the patient; identifying, based on the image, a plurality of immune cells in the image; identifying, based on the image, an epithelium-stroma interface (ESI) separating tumor epithelium and tumor stroma in the image; determining an ESI immune cell density based on the image, wherein the epithelium-stroma immune cell density represents a measure of immune cells within a threshold distance of the ESI; determining immune cell infiltration based on the image, wherein the immune cell infiltration represents a measure of infiltration into the tumor epithelium by immune cells; and predicting the response to the anti-PD-L1 treatment by inputting the ESI immune cell density and the immune cell infiltration into a model.

In some embodiments, the ESI immune cell density comprises a ratio of a number of immune cells within the threshold distance of the ESI and a total number of cells within the threshold distance of the ESI. In some embodiments, the threshold distance is 8 microns. In some embodiments, the immune cell infiltration comprises a ratio between a first ratio and a second ratio, wherein the first ratio is between a number of immune cells and a total number of cells within an area of the tumor epithelium, and wherein the second ratio is between a number of immune cells and a total number of cells within an area of the tumor stroma. In some embodiments, the area of the tumor epithelium is between a first distance and a second distance from the ESI in the tumor epithelium. In some embodiments, the area of the tumor stroma is between the first distance and the second distance from the ESI in the tumor stroma. In some embodiments, the first distance is 24 microns and the second distance is 8 microns.

In some embodiments, identifying the plurality of immune cells comprises: identifying a plurality of cells in the image; and determining whether each cell of the plurality of cells in the image is an immune cell based on a color channel of the image, the color channel highlighting immune cells in the image. In some embodiments, the color channel highlighting immune cells in the image corresponds to a CD8 stain map of the image.

In some embodiments, identifying the ESI in the image comprises: identifying a tumor stroma area in the image. In some embodiments, identifying the tumor stroma area in the image comprises: identifying a first group of pixels in the image based on a luminosity threshold and a color channel distinguishing between the tumor stroma and the tumor epithelium. In some embodiments, the color channel distinguishing between the tumor stroma and the tumor epithelium corresponds to a panCK stain map of the image. In some embodiments, identifying the tumor stroma area in the image further comprises: identifying a second group of pixels in the image based on a plurality of cell nuclei in the tumor stroma according to the color channel distinguishing between the tumor stroma and the tumor epithelium. In some embodiments, the tumor stroma area is a union of the first group of pixels and the second group of pixels.

In some embodiments, the model comprises a machine-learning model or a statistical model. In some embodiments, the model comprises a fitted Cox proportional hazards model.

In some embodiments, predicting the response to the anti-PD-L1 treatment comprises: obtaining a treatment response prediction score from the model; and comparing the treatment response against a predefined threshold. In some embodiments, the anti-PD-L1 treatment comprises: atezolizumab, avelumab, or durvalumab.

According to some embodiments, an exemplary system for determining an immunophenotype of a tumor can comprise: one or more non-transitory computer-readable storage media storing computer program instructions; and one or more processors coupled to the one or more non-transitory computer-readable storage media, the one or more processors being configured to execute the computer program instructions to: receive an image of a tumor of the patient; identify, based on the image, a plurality of immune cells in the image; identify, based on the image, an epithelium-stroma interface (ESI) separating tumor epithelium and tumor stroma in the image; determine an ESI immune cell density based on the image, wherein the epithelium-stroma immune cell density represents a measure of immune cells within a threshold distance of the ESI; determine immune cell infiltration based on the image, wherein the immune cell infiltration represents a measure of infiltration into the tumor epithelium by immune cells; and predict the response to the anti-PD-L1 treatment by inputting the ESI immune cell density and the immune cell infiltration into a model.

According to some embodiments, an exemplary non-transitory computer-readable medium can comprise computer program instructions that, when executed by one or more processors of a computing system, effectuate operations comprising: receiving an image of a tumor of the patient; identifying, based on the image, a plurality of immune cells in the image; identifying, based on the image, an epithelium-stroma interface (ESI) separating tumor epithelium and tumor stroma in the image; determining an ESI immune cell density based on the image, wherein the epithelium-stroma immune cell density represents a measure of immune cells within a threshold distance of the ESI; determining immune cell infiltration based on the image, wherein the immune cell infiltration represents a measure of infiltration into the tumor epithelium by immune cells; and predicting the response to the anti-PD-L1 treatment by inputting the ESI immune cell density and the immune cell infiltration into a model.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example interaction system for generating and processing digital pathology images, in accordance with various embodiments.

FIG. 2 illustrates an example digital pathology image generation subsystem, in accordance with various embodiments.

FIG. 3 illustrates an example epithelium/stroma image processing subsystem, in accordance with various embodiments.

FIG. 4 illustrates an example tile generation module, in accordance with various embodiments.

FIG. 5 illustrates an example tile analysis module, in accordance with various embodiments.

FIG. 6 illustrates an example inflammation type determination module, in accordance with various embodiments.

FIG. 7 illustrates an example stroma criterion determination module/epithelium criterion determine module, in accordance with various embodiments.

FIG. 8 illustrates an example color deconvolution module, in accordance with various embodiments.

FIG. 9 illustrates an example correction factor module, in accordance with various embodiments.

FIG. 10 illustrates an example immunophenotype classification module, in accordance with various embodiments.

FIG. 11 illustrates an example model training process implemented by a model training subsystem, in accordance with various embodiments.

FIG. 12 illustrates an example epithelium-stroma interface image processing subsystem, in accordance with various embodiments.

FIG. 13 illustrates an example tile analysis module, in accordance with various embodiments.

FIG. 14 illustrates an example epithelium-stroma interface detection module, in accordance with various embodiments.

FIG. 15 illustrates an example epithelium-stroma interface immune cell density determination module, in accordance with various embodiments.

FIG. 16 illustrates an example immune cell infiltration determination module, in accordance with various embodiments.

FIG. 17 illustrates an example immunophenotype classification module, in accordance with various embodiments.

FIG. 18A illustrates a flowchart of an example method for performing immunophenotyping using an epithelium/stroma image processing system, in accordance with various embodiments.

FIG. 18B illustrates a flowchart of another example method for performing immunophenotyping using an epithelium-stroma interface image processing subsystem, in accordance with various embodiments.

FIG. 19A illustrates an exemplary method for predicting a response to an anti-PD-L1 treatment by a patient, in accordance with various embodiments.

FIG. 19B illustrates an exemplary method for predicting a response to an anti-PD-L1 treatment by a patient, in accordance with various embodiments.

FIGS. 20A-20C illustrate example visualizations of immune cell density in a whole slide image, tumor stroma, and tumor epithelium, respectively, in accordance with various embodiments.

FIGS. 21A-21C illustrate example graph-based representations of immunophenotype classifications, in accordance with various embodiments.

FIG. 22 illustrates a plot of images based on an epithelium-immune cell density and a stroma-immune cell density for determining tumor immunophenotypes, in accordance with various embodiments.

FIG. 23 illustrates an example tumor region representing a tumor's epithelium and stroma, in accordance with various embodiments.

FIGS. 24A-24D illustrates example feature correlations, in accordance with various embodiments.

FIG. 25 illustrates plots describe the relationship of relative infiltration with CD8+ immune cells and checkpoint inhibitor status, in accordance with various embodiments.

FIG. 26 illustrates an image depicting a biological sample stained using a panCK-CD8 dual stain, in accordance with various embodiments.

FIG. 27 illustrates an example clustering map generated via a color deconvolution process, in accordance with various embodiments.

FIG. 28A illustrates an example image tile depicting a biological sample stained with multiple stains, in accordance with various embodiments.

FIGS. 28B-28C illustrate example histograms describing an epithelium-immune cell density, an epithelium-stroma interface immune cell density, and a stroma-immune cell density, in accordance with various embodiments.

FIG. 29 illustrates tumor immunophenotypes based on infiltration score (Pes) and epithelium-stroma immune cell density (ESI Density), in accordance with various embodiments.

FIG. 30 illustrates the same data of FIG. 29 computed using HK tumor immunophenotypes biomarkers, in accordance with various embodiments.

FIGS. 31A-31C illustrate example overall survival numbers for patients treated with different therapeutics, in accordance with various embodiments.

FIGS. 32A-32B illustrate distributions of immune cells fit using exponentials, in accordance with various embodiments.

FIG. 33 illustrates an example image depicting a tumor region split into image tiles, in accordance with various embodiments.

FIG. 34A illustrates example feature correlations, in accordance with various embodiments.

FIG. 34B illustrates a UMAP of clusters of features indicating correlations, in accordance with various embodiments.

FIG. 35 illustrates example RNA analysis results, in accordance with various embodiments.

FIGS. 36A-36B illustrates an example whole slide image and a region of interest identified therein, in accordance with various embodiments.

FIGS. 37A-37D illustrate tiles depicting various HK tumor immunophenotypes, in accordance with various embodiments.

FIGS. 38A-38E illustrate an example image, image tiles, and distance maps, in accordance with various embodiments.

FIG. 39 illustrates an example image tile may depicting a biological sample stained with hematoxylin, panCK, and CD8, in accordance with various embodiments.

FIGS. 40A-40B illustrate a fraction of tiles (Fs) classified as being inflamed in the tumor stroma and a fraction of tiles (Fe) classified as being inflamed in the tumor epithelium, in accordance with various embodiments.

FIGS. 41A-41B illustrate a flow diagram for patient enrollment in the study for the training set, in accordance with various embodiments.

FIGS. 42A-42J provide exemplary visualization of the digital pathology analysis process, in accordance with various embodiments.

FIGS. 43A-43D provide exemplary visualization of the ESI immune cell density TESI and the immune cell infiltration T_{inf} in a sample region of interest, in accordance with various embodiments.

FIG. 44A-44B illustrate the Spearman correlation coefficients between TESI, T_{Inf}, and measures of CD8+ cell density in the tumor overall, in the intra-tumoral stroma, and in the tumor epithelium in the training set and the validation set, in accordance with various embodiments.

FIG. 45A-45B illustrate the distribution of exemplary TESI and T_{Inf} values, in accordance with various embodiments.

FIG. 46A-46D illustrate exemplary RNA-seq gene set analyses, in accordance with various embodiments.

FIG. 47A-47B illustrate overall survival for patients in an exemplary validation set, in accordance with various embodiments.

FIG. 48A-48C illustrate performance of DACTI, in accordance with various embodiments.

FIG. 49 illustrates an example computer system used to implement some or all of the techniques described herein.

### DETAILED DESCRIPTION

Described herein are systems, methods, and programming describing a localized approach to determining an immunophenotype of a tumor in a digital pathology image based on spatial information of biological objects (e.g., stroma regions, epithelium regions, or immune cells) across all or part of the image. Localized metrics may be generated by identifying these biological objects, dividing the image into tiles, and characterizing the spatial distribution and interrelation of the identified biological objects within each tile. The immunophenotype of the tumor lesion may be determined based on the localized metrics. The disclosed techniques may quantitatively characterize the density and distribution of the biological objects (e.g., in a tumor biopsy). The behavior of immune cells (e.g., T-cells) may be determined by taking into account local heterogeneity and/or the dynamics that cause the spatial distribution, and then building highly predictive biomarkers for a patient's response to one or more IO therapeutics.

Cancer immunology has revolutionized many different cancer treatments, including non-small cell lung cancer (NSCLC). Unfortunately, most of these lung cancer patients do not respond to immunotherapy. As a result, lung cancer remains the leading cancer killer in the United States, causing over 130K deaths each year.

Cancer immunotherapy refers to a technique that harnesses a patient's own immune system to eliminate and/or prevent further recurrences of tumors. Immunotherapies generally include stimulating/boosting the body's natural defenses to work harder and smart to attack cancer cells or therapeutics used to restore/improve the body's immune system. An example of the latter includes checkpoint inhibitors (CIs). CIs refer to drugs designed to restore the immune system's ability to recognize and attack cancer cells. The immune system is designed to differentiate between normal cells and abnormal cells, such as germs, bacteria, and/or cancer cells. This differentiation allows the immune system to effectively attack abnormal cells. This differentiation also prevents the immune system from attacking normal cells.

The immune system performs the aforementioned cell differentiation using a variety of techniques, one of them being “checkpoint” proteins. These checkpoint proteins can switch on/off the immune system's response. Unfortunately, cancer cells can also figure out how to use these checkpoints to avoid the immune system's attacks.

Medicines exist that can block checkpoint proteins made by some types of immune system cells, such as T cells and/or some cancer cells. These checkpoint proteins can assist in keeping immune responses from being too strong. Additionally, these checkpoint proteins can prevent T cells from killing cancer cells. When these checkpoint proteins are blocked, T cells can kill cancer cells better. Some example checkpoint proteins include PD-1/PD-L1 and CTLA-4/B7-1/B7-2.

To address this, some cancer therapies (e.g., monoclonal antibodies) use immune checkpoint inhibitors. These checkpoint inhibitors do not directly kill cancer cells. Instead, checkpoint inhibitors can enable the immune system to more accurately identify cancer cells and mount an attack against the cancer cells. Therefore, it is an important aspect of cancer research to develop inexpensive, simple, and reproducible biomarkers to identify patients that are most likely to respond to checkpoint inhibition, as well as those patients that may require additional treatments. For example, some patients may require additional therapies to prepare the immune system to attack and kill cancer cells.

One checkpoint inhibition pathway is the PD1-PD-L1 interaction, which prevents cytotoxic T cells from killing tumor cells. This pathway can be a primary cause of a patient's tumor growth. To identify whether the PD1-PD-L1 interaction is the main driver, an immunohistochemistry (IHC) assay may be performed. The IHC assay may stain for the PD-L1 or PD1 molecule. A positive result may indicate that the primary driver of tumor growth is the PD1-PD-L1 interaction. Patients whose IHC assay yields a positive result are expected to respond to therapies that disrupt the PD1-PD-L1 interaction, such as atezolizumab or nivolumab. Indeed, PD-L1 IHCs was found to be predictive for checkpoint inhibition therapy in many cancers, such as NSCLC.

Some patients, however, do not respond to therapies that disrupt the PD1-PD-L1 interaction even though those patients' IHC assays are PD-L1 positive. Further complicating matters is that some patients who respond to therapies that disrupt the PD1-PD-L1 interaction have IHC assays that are PD-L1 negative. As an example, a clinical trial where patients were treated with either atezolizumab or docetaxel found that PD-L1 was not predictive for an atezolizumab response at a statistically significant level.

There are numerous theories as to why some patients do not respond to checkpoint inhibitors. One example relates to the cancer cells themselves, which can have low immunogenicity (e.g., the ability to produce an immune response to a pathogen). Cancer cells can also increase regulatory T cell (Tregs) production, which function to suppress immune response. Cancer cells can also rely on inhibitory signals other than PD1-PD-L1.

Another possible explanation as to why some patients do not respond to checkpoint inhibition therapy may be that there are too few immune cells, which actively kill tumor cells (e.g., CD8 cytotoxic T cells . . . ), near the tumor. Immune cells may also be unable to penetrate the tumor epithelium, instead remaining in the tumor stroma.

These immune cells can positively impact mortality rates for most cancers, such as NSCLC. A patient that does not respond to immunotherapy may not have enough CD8+ T cells in the vicinity of a tumor lesion. Alternatively, or additionally, unknown impediments can prevent T cells from reaching target tumor cells in a stromal component of a tumor lesion

Therefore, accurately predicting whether a patient will respond to one or more therapies can greatly improve that patient's outcome. Described herein are technical solutions to the aforementioned technical problems, including harnessing digital pathology to develop biomarkers for predicting whether a patient will respond to a particular therapy or therapies.

In some embodiments, digital pathology may be used to characterize a density, distribution, or other characteristics of tumor cells. From these characteristics, parameters may be extracted that can predict whether a patient will respond to a particular therapy (e.g., atezolizumab). Some embodiments include determining T cell behavior using local heterogeneity or the dynamics that lead up to the T cells distribution. These techniques may enable predictive biomarkers to be developed for identifying whether a patient will respond to the particular therapy.

The process of quantifying immune cell (e.g., CD8+ T-cell) infiltration into tumors includes quantifying a density of immune cells in a tumor epithelium or in the tumor stroma, and then averaging the two. However, no existing clinical trials employ the immune cell infiltration quantification mentioned above for analysis of non-small cell lung cancer (NSCLC). Part of the reason for this is that the heterogeneity of immune cell distributions was extensive for lung cancer studies. Averaging the densities across the entire tissue sample may not capture the localized effects of the immune cell distribution.

To capture immune cell densities while accounting for heterogeneity (e.g., no averaging across the slide), the present disclosure may include embodiments in which an image of the tissue sample may be divided into image tiles (also referred to as tiles). The immune cell density may be computed at an image tile-level so as to now capture localized effects.

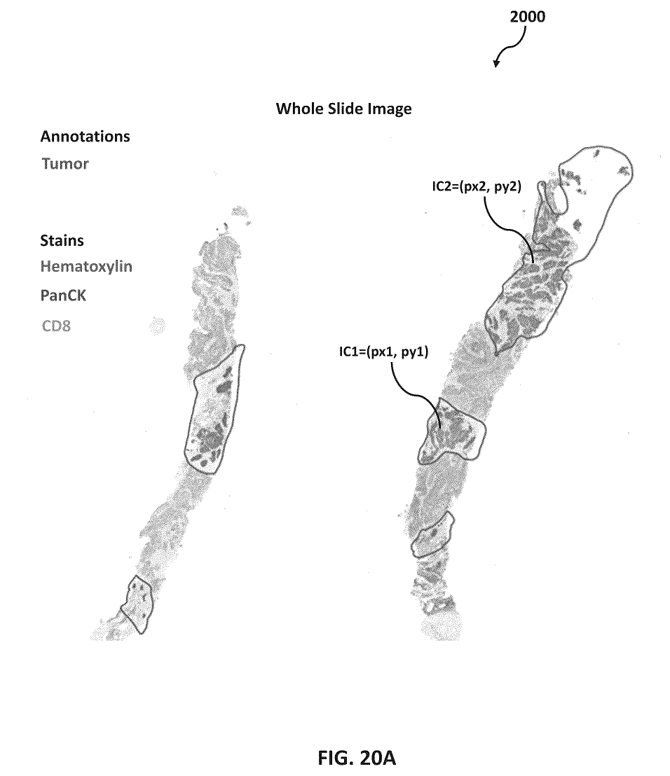

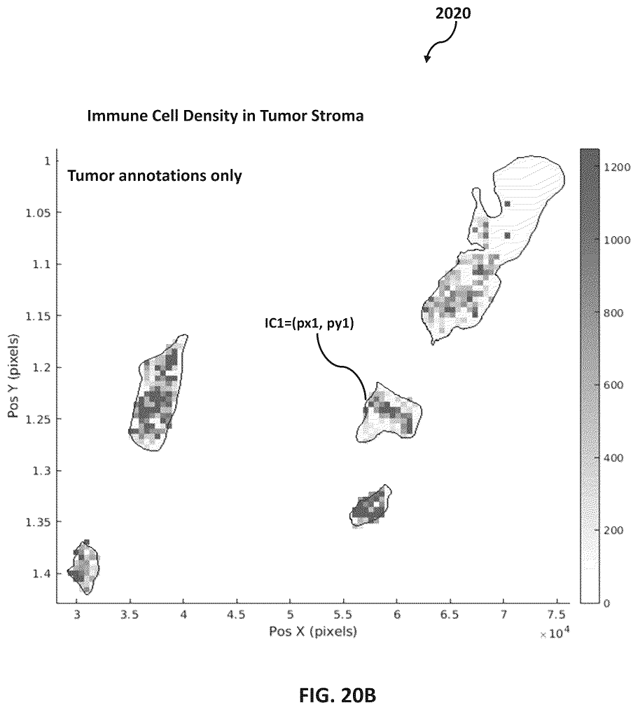

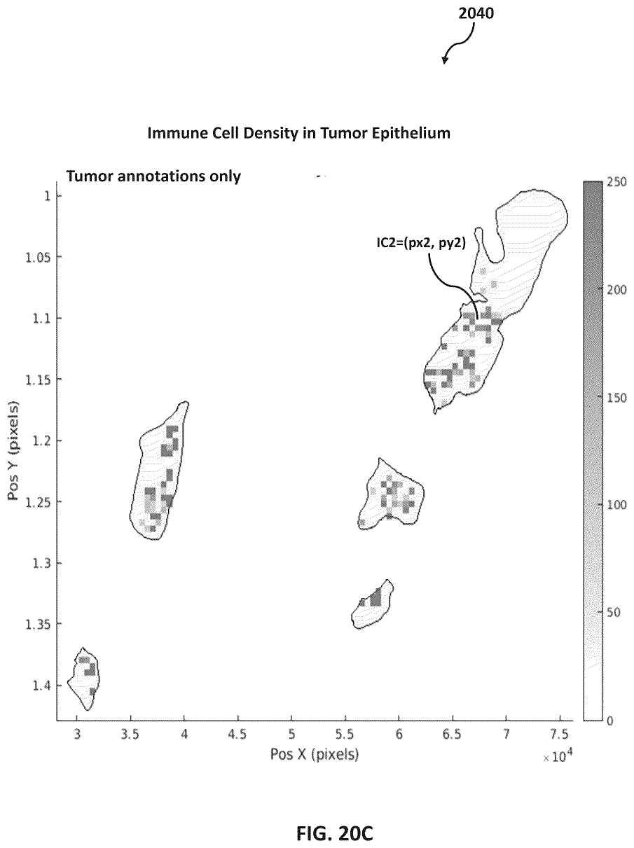

In some embodiments of the present disclosure, the fraction of tiles. Fs, where the density of the immune cells in the tumor stroma was greater than a threshold density and/or the fraction of tiles. Fe, where the density of the immune cells in the tumor epithelium was greater than a threshold density, can be computed for the whole slide image. This may be done instead of averaging these densities over the whole slide image. An example visualization and quantification of the immune cell distribution are illustrated in FIGS. 20A-20C.

In embodiments of the present disclosure, different immunophenotypes may be assigned to whole slide images based on an infiltration type of the tumor. For example, three immunophenotypes may include “inflamed.” “excluded.” and “desert.” An immunophenotype classification of inflamed refers to (i) a number of tiles of a first inflammation type being greater than or equal to a first threshold and (ii) a number of tiles of a second inflammation type being greater than or equal to a second threshold. The inflammation type may be a first inflammation type or a second inflammation type. The first inflammation type may be based on a first stroma criterion for a stroma-immune cell density and a second stroma criterion for an epithelium-immune cell density being met. The second inflammation type may be based on a first epithelium criterion for a stroma-immune cell density and a second epithelium criterion for an epithelium-immune cell density being met. The first stroma criterion for the stroma-immune cell density being met may include the stroma-immune cell density being greater than or equal to a stroma-immune cell density threshold. The second stroma criterion for the epithelium-immune cell density being met may include the epithelium-immune cell density being less than or equal to an epithelium-immune cell density threshold. The first epithelium criterion for the stroma-immune cell density being met may include the stroma-immune cell density being less than the stroma-immune cell density threshold. The second epithelium criterion for the epithelium-immune cell density being met may include the epithelium-immune cell density being less than the epithelium-immune cell density threshold. An immunophenotype classification of excluded refers to (i) a number of tiles of the first inflammation type being greater than or equal to the first threshold and (ii) a number of tiles of the second inflammation type being less than the second threshold. An immunophenotype classification of desert refers to a number of tiles of the first inflammation type being less than the first threshold, while a number of tiles of the second inflammation type being less than the second threshold (e.g., low Fs and low Fe). The number of tiles of the first inflammation type and the number of tiles of the second inflammation type may correspond to tiles from an image (e.g., a whole slide image) of a biological sample (e.g., a tumor including tumor stroma and tumor epithelium).

It is to be understood that in some embodiments, the various immunophenotype classifications, stroma criteria, and epithelium criteria, may be measured by any type of thresholds and are not limited to the comparative values listed above. For example, in some embodiments, the first stroma criterion for the stroma-immune cell density being met may include the stroma-immune cell density being less than a stroma-immune cell density threshold (instead of greater than or equal to the stroma-immune cell density threshold, as described above).

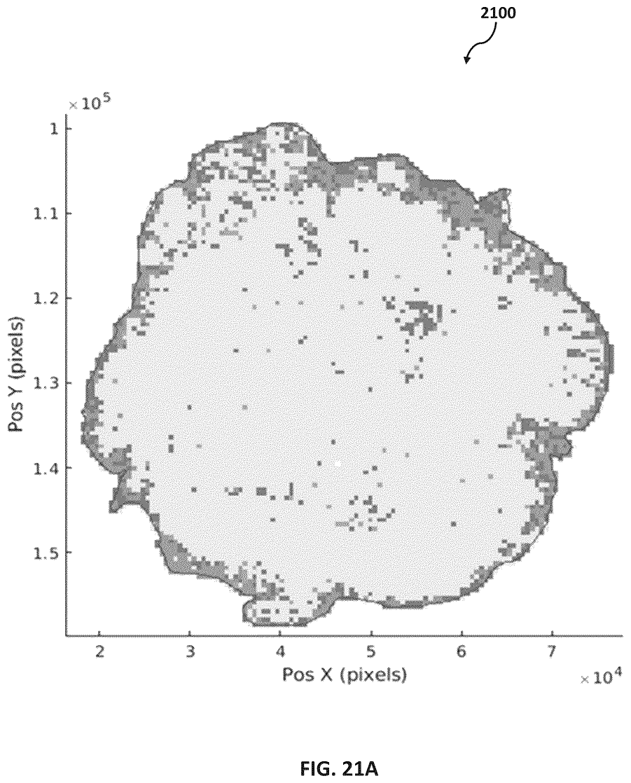

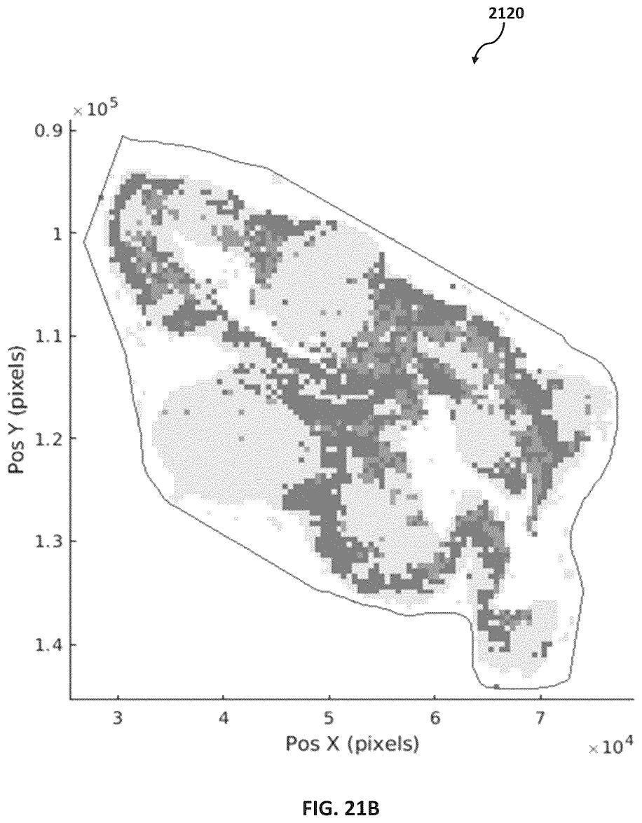

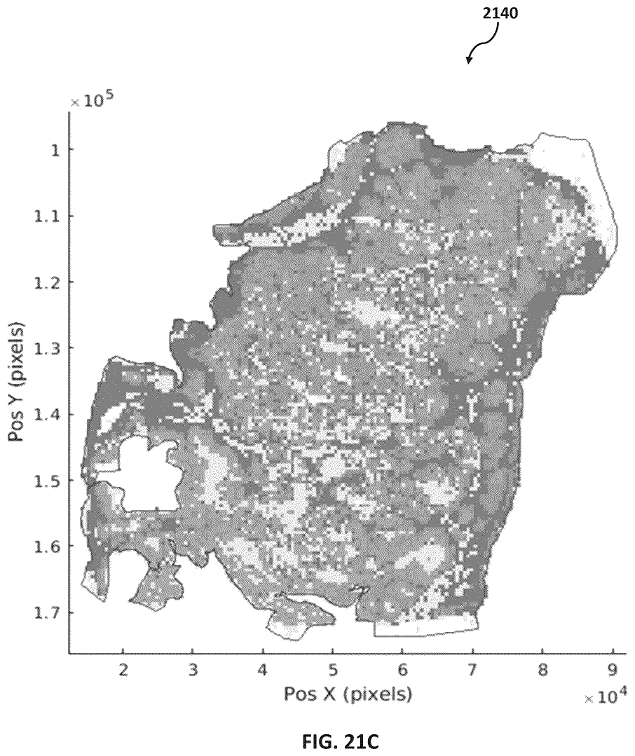

As an example, with reference to FIGS. 21A-21C, whole slide images 2100, 2120, and 2140 depict different immunophenotype classifications based on the density of the immune cells in tumor stroma and tumor epithelium. For instance, whole slide image 2100 of FIG. 21A depicts an example of a desert immunophenotype classification. In whole slide image 2100, Fs and Fe may both be low, indicating that the immune cell density in the tumor stroma and the tumor epithelium is low. Whole slide image 2120 of FIG. 21B depicts an example of an excluded immunophenotype classification. In whole slide image 2120, Fe may remain low, however Fs may be high, indicating that the immune cell density in the tumor stroma is high. Whole slide image 2140 of FIG. 21C depicts an example of an infiltrated or inflamed immunophenotype. In whole slide image 2140, Fe and Fs may be high, indicating that the immune cell density in the tumor stroma and the tumor epithelium is high.

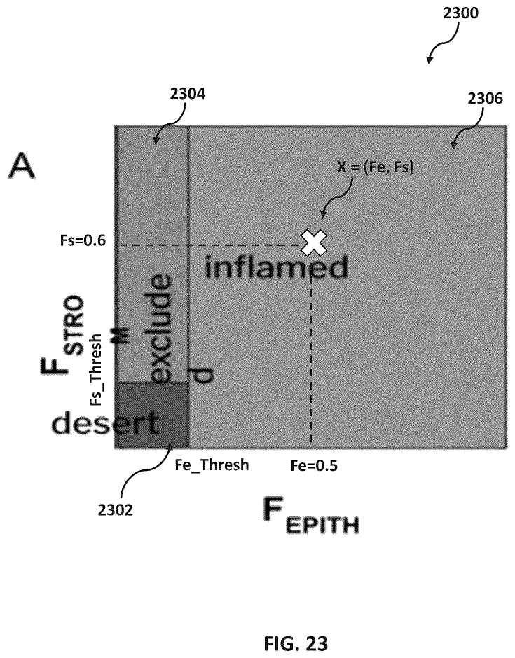

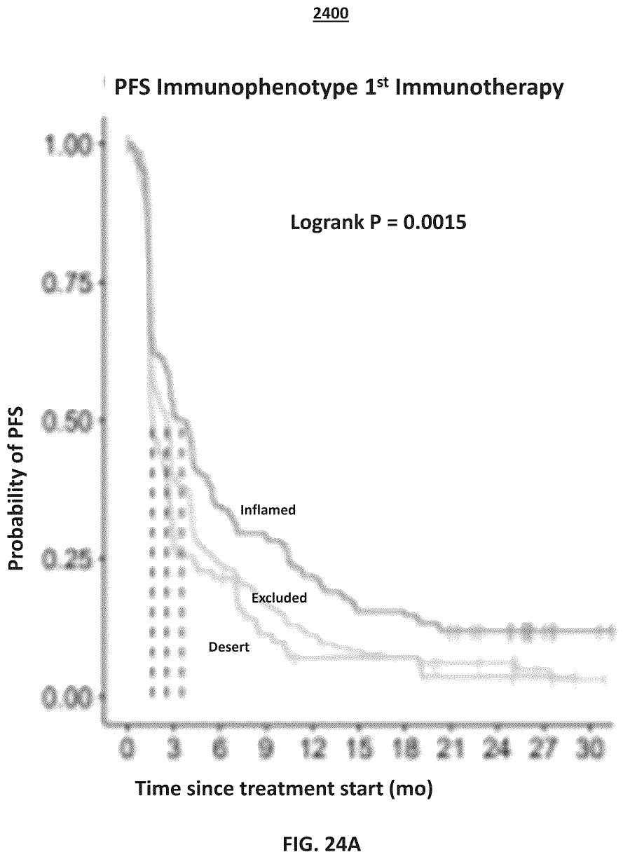

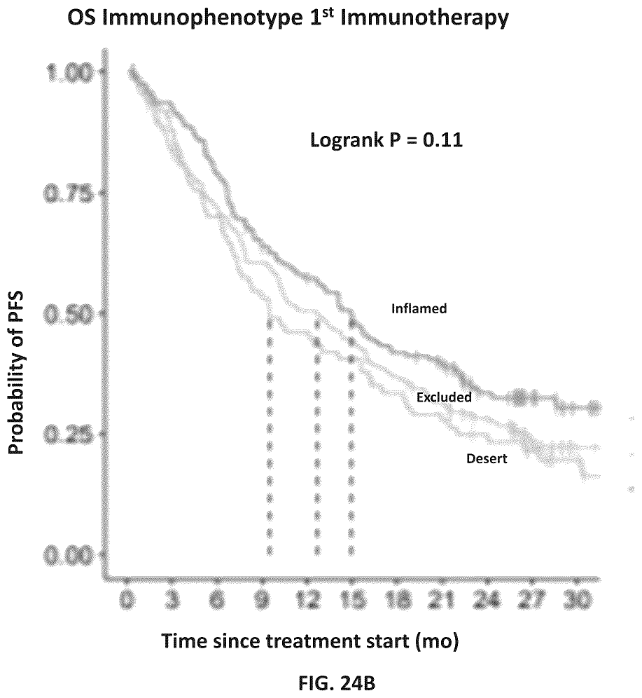

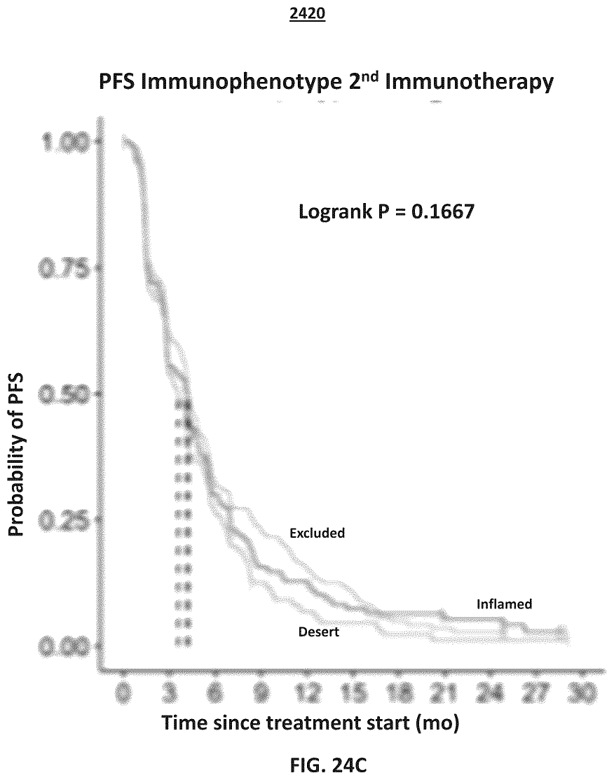

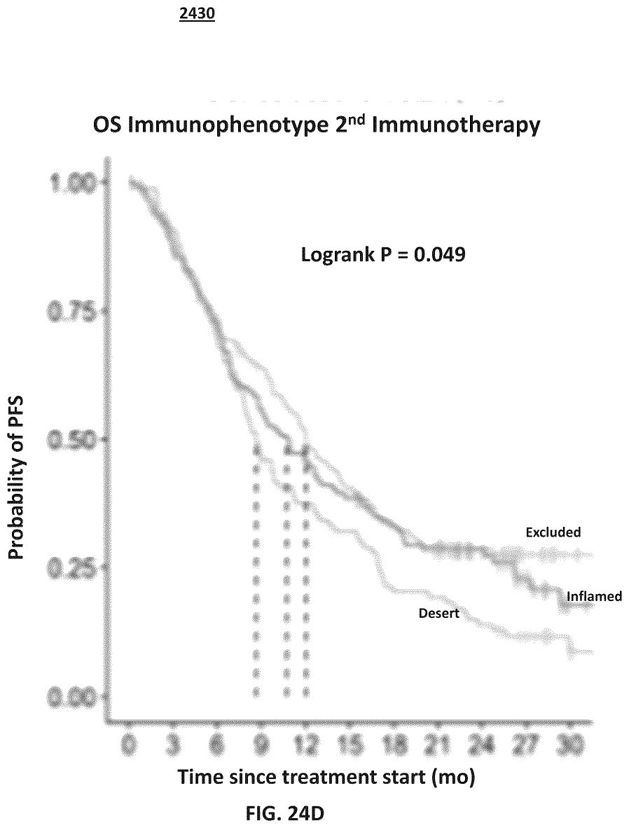

In some embodiments, a graph describing each patient's biological samples may be generated. As an example, plot 2200 of FIG. 2200 may depend on the number of tiles of the first inflammation type and/or the number of tiles of the second inflammation type. In plot 2200, the HK tumor immunophenotype classifications for patients may be plotted as a function of the epithelium-immune cell density and the stroma-immune cell density. In some embodiments, thresholds may be set for the immunophenotype classifications. For example, tumor immunophenotype classification data 2300 of FIG. 23 may indicate the values of/'s and F′e and the corresponding immunophenotype classifications associated with these values. In tumor immunophenotype classification data 2300, region 2302 (in red) may represent the immunophenotype classification of desert, region 2304 (in green) may represent the immunophenotype classification of excluded, and region 2306 (in blue) may represent the immunophenotype classification of inflamed. Survival plots for the whole patient population may also be produced, as illustrated by plots 2400-2430 of FIGS. 24A-24D.

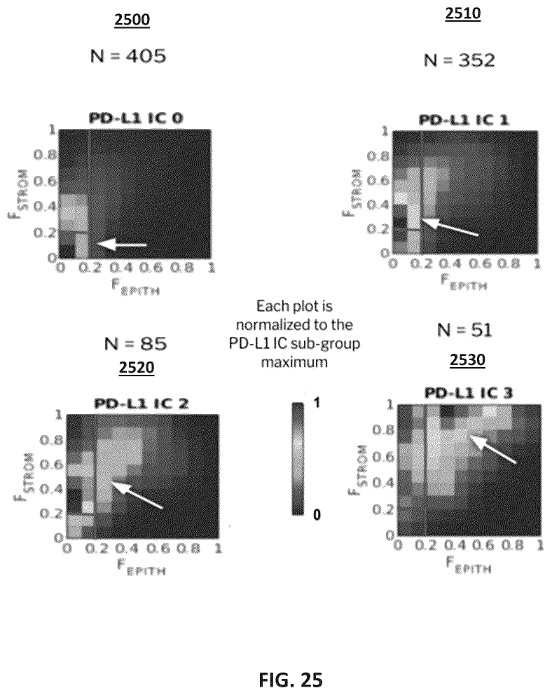

In some embodiments, sub-groups of patients may be identified, where each sub-group of patients indicates patients who were predictive of a response to a particular immunotherapy (e.g., atezolizumab, docetaxel). To identify the sub-groups of patients, a relationship between relative immune cell infiltration and checkpoint inhibitor status (e.g., PD-L1) must be established. As an example, with reference to FIG. 25, plots 2500-2530 describe the relationship of the relative infiltration with CD8+ immune cells and checkpoint inhibitor status. In FIG. 25, the variable N represents a number of patients and IC represents an immune cell score. As seen by plots 2500-2530, as the immune score IC increases, first the stromal compartment becomes more infiltrated (e.g., plots 2500-2510) and later the epithelial compartment as well (e.g., plots 2520-2530).

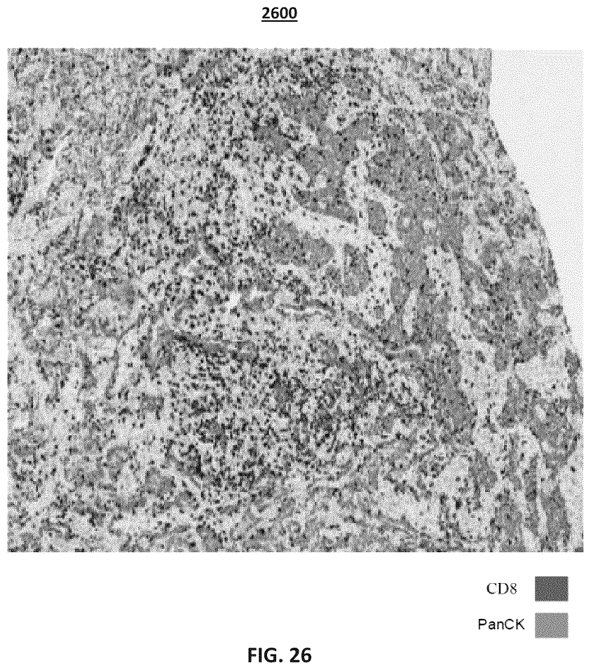

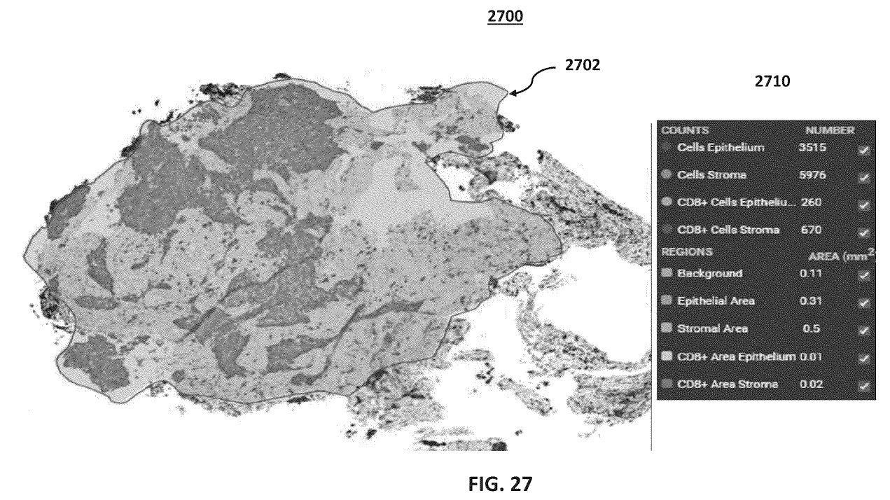

CD8+ immune cells relate to immune cells stained by the CD8 stain. CD8+ stains appear brown on the IHC image. CD8− (or CD8 negative) immune cells refer to immune cells that are not stained by the CD8 stain. For example, as seen in FIGS. 26 and 27, images 2600 and 2700 each depict a biological sample stained with the CD8 stain (highlighting immune cells in brown) and the panCK stain (highlighting tumor epithelium in pink). The whole tumor area depicted in images 2600 and 2700 may be determined by pathologists. These pathologists may manually annotate the images to depict the region of the whole slide image describing the tumor (e.g., tumor stroma and tumor epithelium). For example, as seen in FIG. 27, border 2702 may depict a border of a tumor. In some embodiments, computer vision models may be used (alone or in conjunction with pathologist input) to determine border 2702, as well as other biological structures depicted within whole slide image 2700.

In some embodiments, an immunophenotype of a tumor may be determined by analyzing an immune cell density at an interface of the tumor stroma and tumor epithelium. An image (e.g., a whole slide image) of a tumor may be received. Based on the image, an epithelium-stroma interface may be identified. The epithelium-stroma interface separates tumor epithelium and tumor stroma. In some embodiments, the epithelium-stroma interface may be identified based on the various color channels forming the image. For example, the tumor may be stained using one or more staining agents each configured to highlight different components of the tumor (e.g., a pan-cytokeratin (panCK) stain for highlighting tumor epithelium, a cluster of differentiation 8 (CD8) stain for highlighting immune cells (e.g., T cells), a hematoxylin stain for highlighting cell nuclei, an extracellular matrix, and/or cell cytoplasm). Separating the image into the different color channels may improve the ability to accurately identify tumor epithelium, tumor stroma, the epithelium-stroma interface separating the tumor epithelium and tumor stroma, immune cells, and other information. In some embodiments, one or more machine learning models (e.g., computer vision models) may analyze the color channels to identify the epithelium-stroma interface and the immune cells.

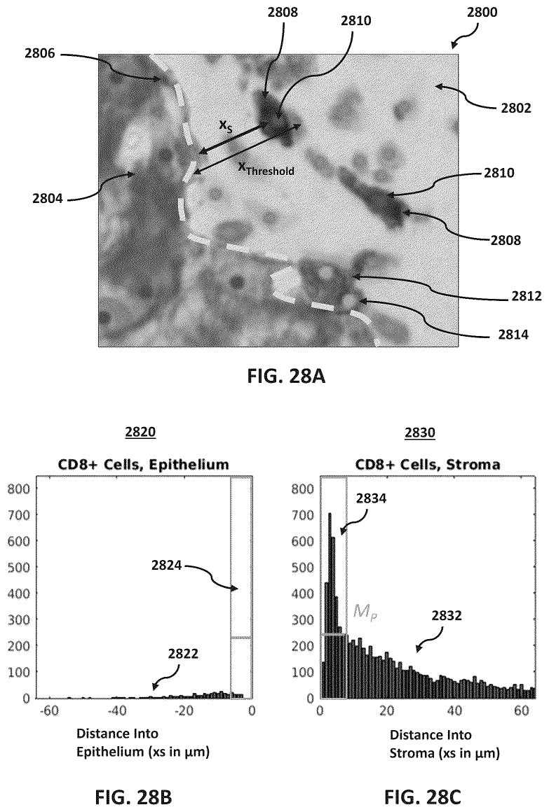

In some embodiments, an epithelium-stroma immune cell density M_{P} may be determined based on the image. The epithelium-stroma immune cell density M_{P} may represent a quantity of immune cells within a threshold distance of the epithelium-stroma interface. For example, with reference to FIG. 28A, immune cells 2808 may be identified within tile 2800. If immune cells 2808 are determined to be within a threshold distance x_{Threshold} of epithelium-stroma interface 2806, then those immune cells 2808 may be included in the computation of the epithelium-stroma immune cell density M_{P}. For example, immune cell 2808 may be a distance xₛ from epithelium-stroma interface 2806 where distance xₛ is less than threshold distance x_{Threshold}, and therefore immune cell 2808 may be included in the determination of the epithelium-stroma immune cell density M_{P}. The quantity of immune cells detected within tumor stroma 2802, tumor epithelium 2804, and at epithelium-stroma interface 2806 is illustrated in histograms 2820 and 2830 of FIGS. 28B and 28C.

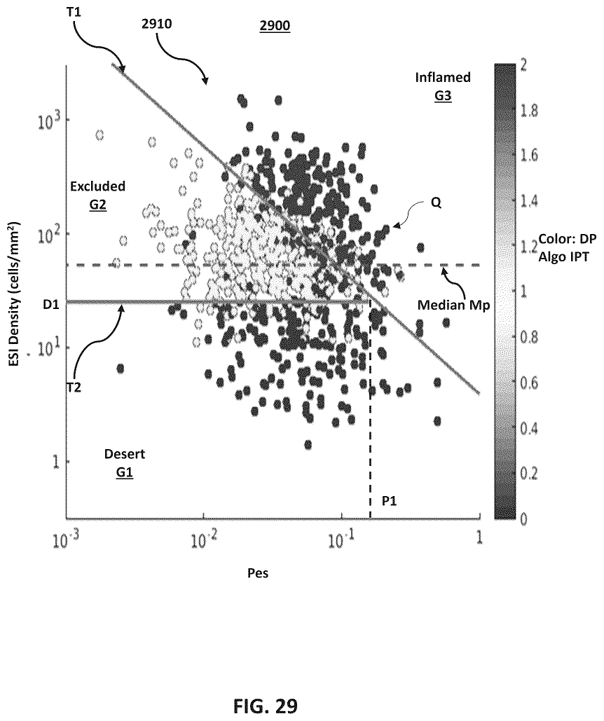

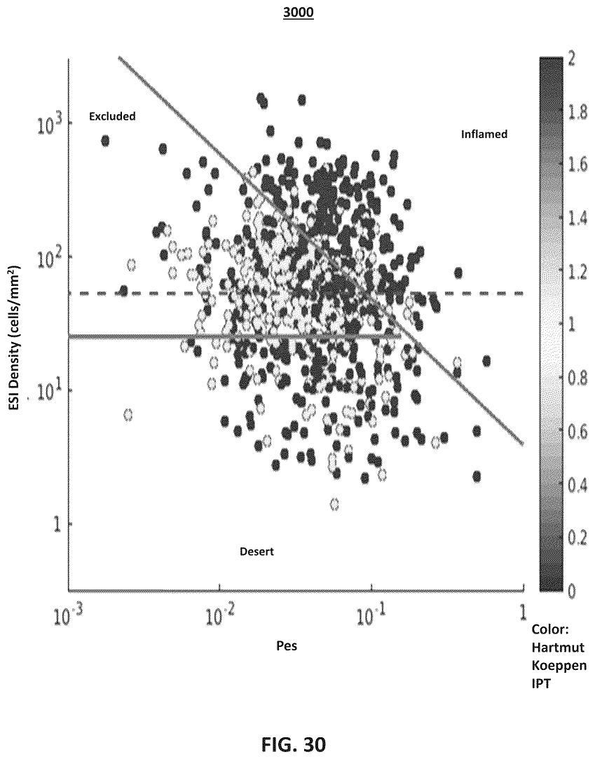

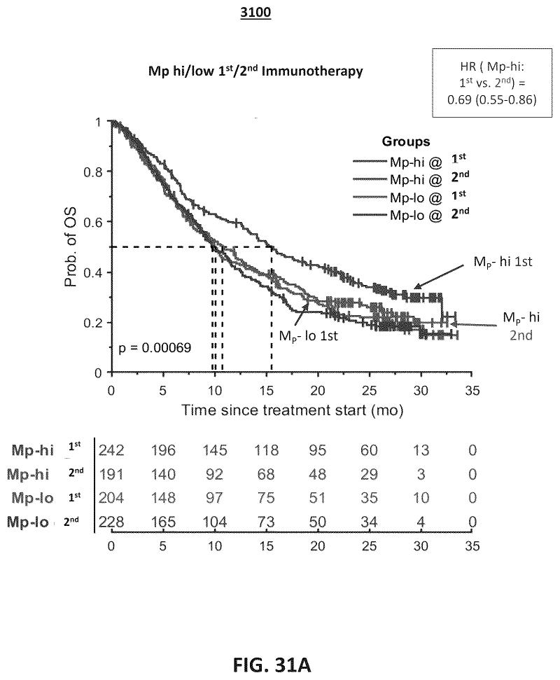

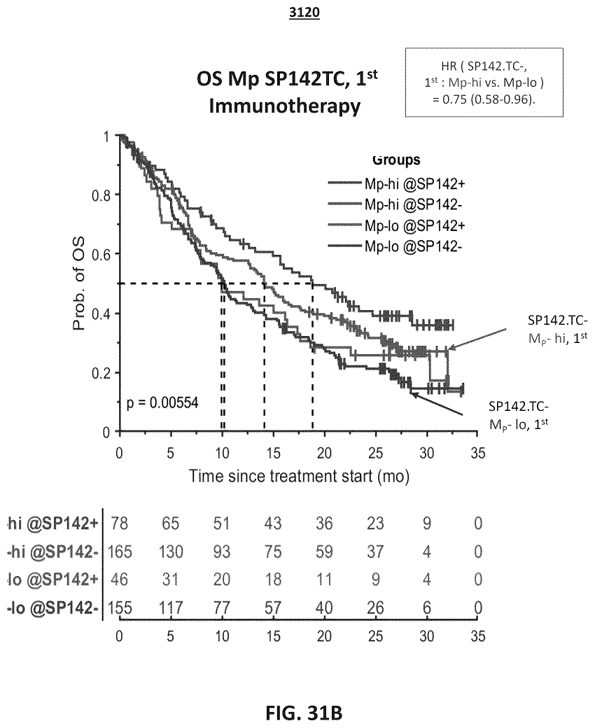

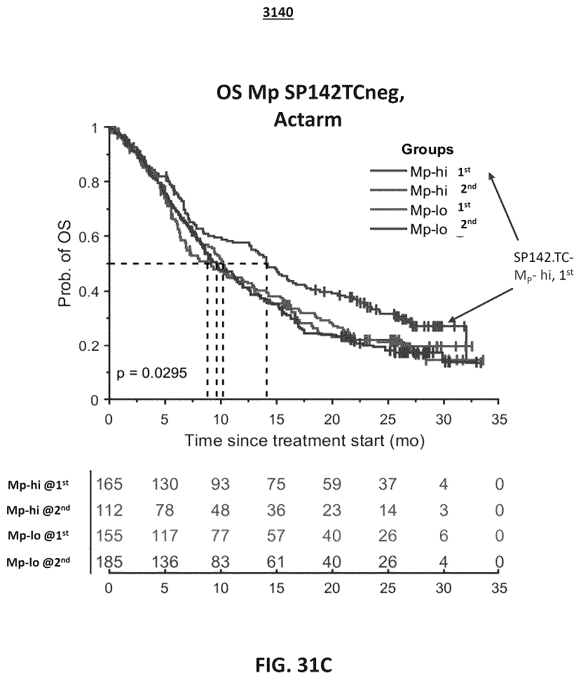

An immune cell infiltration probability may be determine based on a number of immune cells in the tumor stroma and a number of immune cells in the tumor epithelium. The immune cell infiltration probability may indicate a likelihood that an immune cell in the tumor stroma will infiltrate the tumor epithelium. Immune cells in the tumor epithelium can attack cancer cells, thus the more immune cells in the tumor epithelium the greater a patient's outcome can be. In contrast to the previously described technique where an epithelium-immune cell density and a stroma-immune cell density are determined when immunophenotyping, some embodiments may alternatively (or additionally) determine an epithelium-stroma immune cell density for the epithelium-stroma interface. The tumor immunophenotype may be determined based on the epithelium-stroma density and the immune cell infiltration probability. For example, with reference to FIG. 29, plot 2900 depicts data indicating tumor immunophenotypes based on infiltration score (Pes) and epithelium-stroma immune cell density (ESI Density). In some embodiments, the median epithelium-stroma immune cell density may function as a predictive biomarker. As indicated by plots 2900 and 3000 of FIGS. 29-30, use of the median epithelium-stroma density Mp as a predictive biomarker instead of or in addition to the Hartmut Koeppen immunophenotypes can result in identification of a same overall survival but with more patients being recommended for treatment. For example, with reference to Table 1 below and plots 3100-3140 of FIGS. 31A-31C, the median epithelium-stroma density Mp may behave the same or similar to the predictive biomarker used for certain clinical trials (e.g., non-small cell lung cancer clinical trials).

TABLE 1

| Criteria: | IP inflamed | >Median M_{P} |
|---|---|---|
| OS/months | 15.6 | 15.5 |
| Patients: N/Nₜₒₜ | 135/390 = 34% | 242/444 = 55% |

Described herein are technical solutions to the aforementioned technical problems. Some embodiments include determining whether there exists an optimized digital pathology marker that can be used.

As mentioned above, immune cells need to infiltrate the tumor epithelium to attack and kill the cancer cells. If the immune cells are not at the epithelium-stroma interface (ESI), then the immune cells have to travel to get into the tumor epithelium. In some embodiments, an immune cell's behavior can be characterized by the distribution of the immune cells at various distances from the epithelium-stroma interface (ESI). As an example, tile 2800 of FIG. 28A depicts epithelium-stroma interface (ESI) 2806 representing the boundary between tumor epithelium 2804 and the surrounding tumor stroma 2802. The behavior of an immune cell 2808 may be characterized based on the distribution of immune cells as a function of distance xₛ from ESI 2806. Features can be identified/extracted that can capture and predict a patient's response to an immunotherapy.

In some embodiments, there may be a gradient of attraction coming from the tumor cells. The gradient of attraction may be described by a particular shape including three separate distributions. Each distribution represents the distribution of immune cells in a particular region of the tumor. For example, a first distribution 2822 of histogram 2820 of FIG. 28B may correspond to a region inside the tumor epithelium (e.g., tumor stroma 2804), a second distribution 2832 of histogram 2830 of FIG. 28C may correspond to a region outside the tumor epithelium (e.g., tumor stroma 2802), and a third distribution including a portion 2824 in histogram 2820 of FIG. 28B and a portion 2834 in plot 2830 of FIG. 28C may correspond to a region at ESI 2806 (e.g., within a threshold distance x_{Threshold} of ESI 2806).

In some embodiments, the characteristics of the various immune cell distributions may be estimated using one or more predictive models. In some embodiments, the predictive models indicate that solutions to the diffusion equations are a class of functions that describe the T cell distribution in tumor stroma and tumor epithelium. The predictive models may use one or more assumptions.

For example, one assumption for the predictive model may be that a chemokine gradient from the ESI to which the immune cells (e.g., T cells) are attracted may be an input condition for the predictive model. A “chemokine” refers to any class of cytokines whose functions include attracting white blood cells (i.e., immune cells) to infection sites. The chemokine gradient may be referred to as a “stickiness” factor at the ESI. In other words, when the immune cells are in contact with tumor cells. After the immune cells reach the ESI, the stickiness factor may operate to reduce a likelihood that the immune cells will diffuse back into the tumor stroma. Within the tumor epithelial, immune cells may have a substantially even concentration of chemokine and may not be attracted in any particular direction. Therefore, the immune cells within the tumor epithelial can diffuse randomly.

An example assumption for the predictive model may be that there is a constant rate of immune cells entering the stroma. Another example assumption for the predictive model may be that all of the immune cells are said to be entering the stroma. An origin of the immune cells can be considered as a farthest distance from the ESI. Yet another example assumption may be that there is only a finite number of immune cells entering the tumor stroma. For example, the rate at which immune cells replicate or die within the tumor epithelium is assumed to be very low compared to the rate at which the immune cells travel to the ESI and into the tumor epithelium. As another example, there may be no immune cell loss, and a number of iterations of the model is a free parameter.

Still other assumptions may include that there may be a repellent component in the vicinity of the ESI that can slow down the diffusion of immune cells towards the ESI, and that the diffusion rate in the tumor epithelium can be different than the diffusion rate in tumor stroma.

The behavior of the immune cells can be described with three phases. A first phase may correspond to movement of the immune cells towards the ESI. The first phase may indicate whether the stickiness factor attracts cells or repels cells. A second phase may correspond to the immune cells transitioning into the tumor epithelium. The tumor epithelium has a high level of stickiness, and therefore the immune cells may not be able to turn back and exit. A third phase may correspond to movement of the immune cells within the tumor epithelium. In the tumor epithelium, the immune cells may have reduced motility.

In some embodiments, the predictive model may be developed with one or more boundary conditions. These boundary conditions may include a start position of the immune cells in the tumor stroma being initially set at a maximum distance from the ESI. These boundary conditions may also include that there are a finite number of immune cells that will enter the tumor stroma. These boundary conditions may also include that there are a finite number of steps or iterations. These boundary conditions may further include that immune cells are not removed, as steady state has not been reached. Finally, the boundary conditions may include those interactions with chemokine gradients, attractants, and/or repellents may be modified by modifying a likelihood of immune cell advancement or retreat.

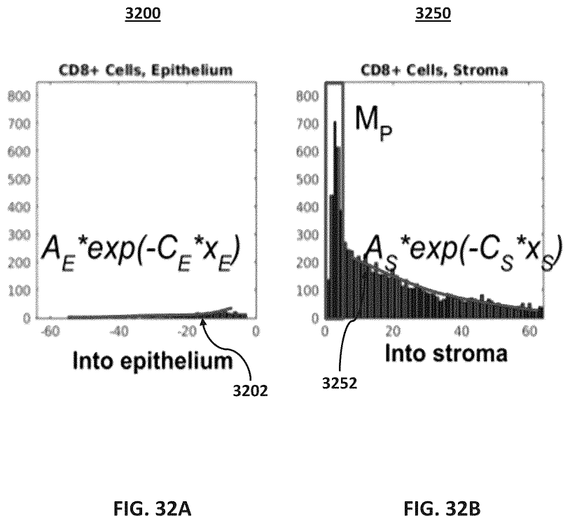

In some embodiments, an exponential curve fitting of the various distributions of the immune cells may be computed, as illustrated in FIGS. 32A and 32B. In some embodiments, an epithelium-immune cell distribution 3202, representing an epithelium-immune cell density of first distribution 2822, may be described by Equation 1:

$${A}_{E} * exp ( - {C}_{E} * {x}_{E} ) . \qquad Equation 1$$

In Equation 1, the term AF may represent an epithelium-immune cell amplitude, and the term C_{E} may represent an epithelium constant (e.g., decay/growth constant). In some embodiments, a stroma-immune cell distribution 3252, representing an epithelium-immune cell density of distribution 2832, may be described by Equation 2:

$${A}_{S} * exp ( - {C}_{S} * {x}_{S} ) . \qquad Equation 2$$

In Equation 1, the term A_{S} may represent a stroma immune cell amplitude, and the term C_{S} may represent a stroma constant (e.g., decay/growth constant).

In some embodiments, the epithelium-immune cell density may be computed by determining a number of immune cells within bins included in first distribution 2822 of FIG. 28B. The stroma-immune cell density may be computed based on a number of immune cells within bins included in distribution 2832 of FIG. 28C.

In some embodiments, an epithelium-stroma immune cell density may be described by a number of immune cells within a threshold distance of the ESI. For example, with reference to FIGS. 28B and 28C, the epithelium-stroma interface immune cell density (Mp) may be computed based on a number of immune cells included in the bins included in portions 2824 and 2834 of the third distribution. The size of each bin may be based on a size of the cells to be identified. For example, with reference to FIG. 28A, immune cells 2808 and 2812 and/or intra-cellular structures 2810 and 2814 (e.g., nuclei) may be approximately 2-10 microns in diameter. The bins included within histograms 2820 and 2830 may have a size of between 2-20 micros. The threshold distance from the ESI may be the same both into the tumor stroma and into the tumor epithelium. For example, the threshold distance may include a first distance from the ESI and a second distance from the ESI. The first distance from the ESI may include a distance from the ESI into the tumor stroma. The second distance from the ESI may include a distance from the ESI into the tumor epithelium. The first distance and the second distance may be the same or different. For example, the first distance and the second distance may be 1 micron or less from the ESI, 2 microns or less from the ESI, 5 microns or less from the ESI, 10 microns or less from the ESI, 20 microns or less from the ESI, or other distances.

In some embodiments, re-labeling or corrections of cell labels may be determined based on a distance of intra-cellular structures 2810 and 2814 to ESI 2806 of FIG. 28A. As an example, with reference to FIGS. 28B-28C, histograms 2820 and 2830 depict a distribution of immune cells (e.g., CD8+ cells) in the tumor epithelium and the tumor stroma, respectively. For instance, in FIG. 28B, as the distance xₛ to ESI 2806 decreases, the number of immune cells 2808 and 2812 may increase. In FIG. 28C, as the distance to ESI 2806 increases, the number of immune cells 2808 and 2812 may decrease.

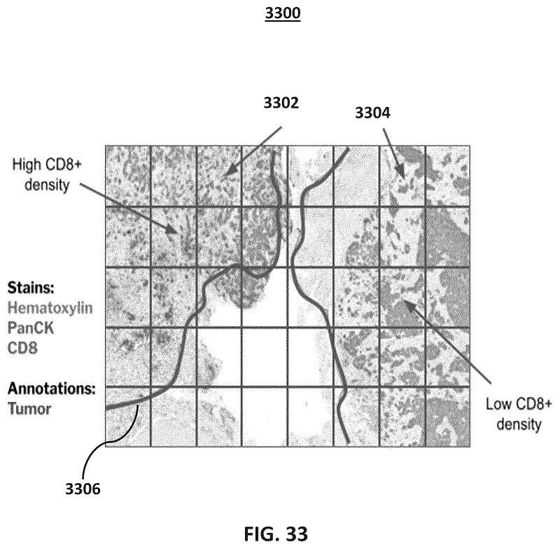

FIG. 33 illustrates an image 3300 depicting a tumor split into image tiles, in accordance with various embodiments. As seen in image 3300), an annotation process may be performed to indicate which portions of image 3300 depict tumor lesions. For example, annotations 3306 may demarcate portions 3302 and 3304, depicting tumor lesions. In some embodiments, annotations 3306 may be added by a human annotator (e.g., a pathologist), whereas other embodiments may implement machine learning techniques to annotate image 3300. In the illustrative example, the immune cells (e.g., CD8+ cells) located in portion 3302 may be more densely concentrated than the immune cells in portion 3304.

In some embodiments, digital pathology parameters were identified that described biologically meaningful phenomena. To identify these digital pathology parameters. RNAseq signatures (e.g., Mp) from a NSCLC clinical trial dataset may be correlated with digital pathology parameters.

Correlating the RNAseq signatures may include selecting a set of RNAseq signatures from the data. Sub-groups may be identified using one or more clustering methods. For example, a uniform manifold approximation and projection (UMAP) clustering technique may be used to identify clusters in the RNAseq data. The UMAP clustering technique allows patterns of clustering to be visualized, which is also focused on local clustering. Differing from other clustering techniques, like tSNE, UMAP clustering does not apply normalization and uses k-nearest neighbors. In the UMAP produced plot, each curated gene expression signature of interest is plotted as a point as the two-dimensional representation of its numerical value across all patients, where each data point may be assigned a color to indicate a classification or grouping among all such signatures evaluated. In some embodiments, UMAP clustering may be performed using various distance measures between gene expression signatures, including but not limited to cosine, Euclidian, and correlation measures.

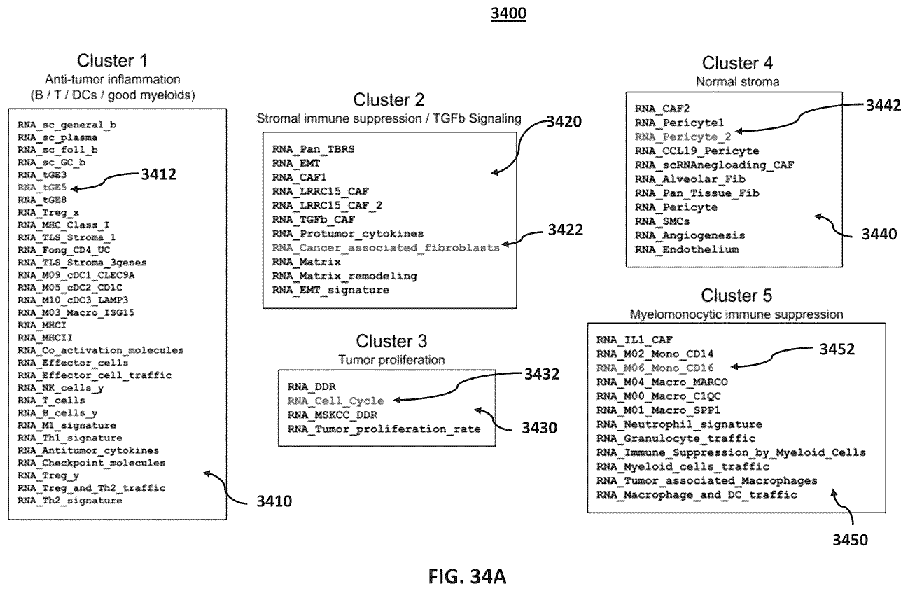

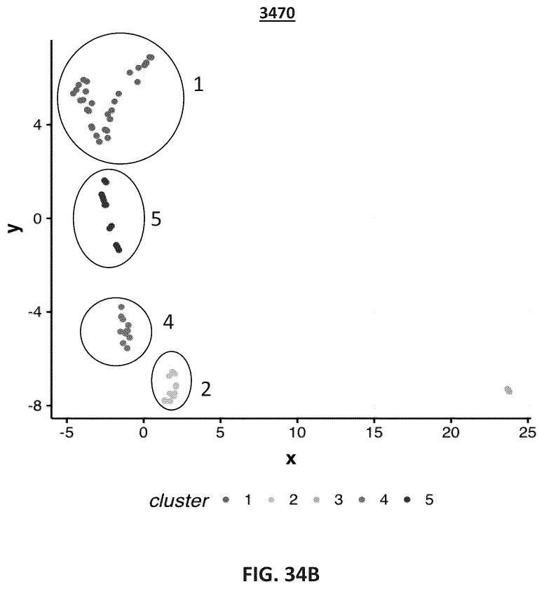

In some embodiments, the different UMAP identified sub-groups may include anti-tumor inflammation (B/T/DCs/good myeloids) sub-group, a stromal immune suppression/TGFb signaling sub-group, a tumor proliferation sub-group, a normal stroma sub-group, a myelomonocytic immune suppression, or other sub-groups. Persons of ordinary skill in the art will recognize that more, fewer, and/or different sub-groups may be used and the aforementioned is merely exemplary. The RNAseq-es identified from the sub-groups may be correlated with various digital pathology features to determine which digital pathology feature is most strongly correlated. As an example, with reference to FIG. 34A, feature correlations 3400 may include the anti-tumor inflammation sub-group represented as “Cluster 1,” which may include DP features 3410. The stromal immune suppression/TGFb signaling sub-group may be represented as “Cluster 2,” which may include DP features 3420. The tumor proliferation sub-group may be represented as “Cluster 3,” which may include DP features 3430. The normal stroma sub-group may be represented as “Cluster 4,” which may include DP features 3440. The myelomonocytic immune suppression sub-group may be represented as “Cluster 5,” which may include DP features 3450. An example UMAP producing Clusters 1-5 is illustrated by plot 3470 of FIG. 34B. Each cluster may include a feature, feature 3412 of DP features 3410, feature 3422 of DP features 3420, feature 3432 of DP features 3430), feature 3442 of DP features 3440), feature 3452 of DP features 3450), that is determined to be most strongly correlated with the RNAseq. The predictive model developed to describe the immune cells distribution indicates that true biological effects are being captured. With this, causes of the various immune cell distributions can further be analyzed. Furthermore, the digital pathology features can be correlated to bulk RNAseq from known biological pathways.

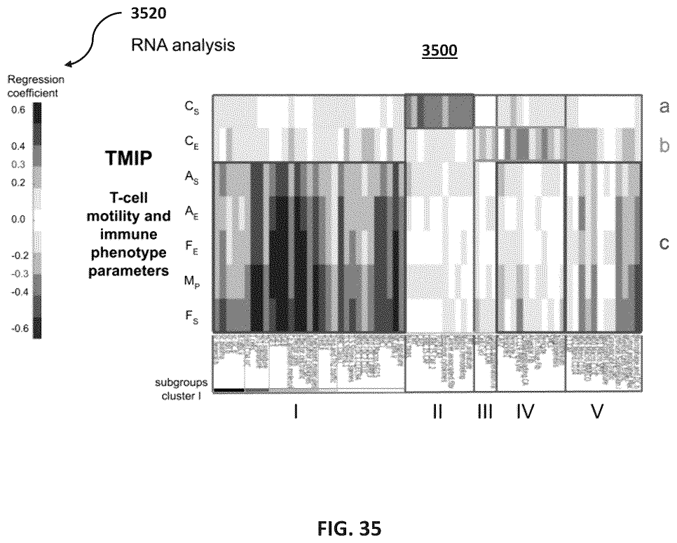

In some embodiments, an RNA analysis may be performed to identify clusters of RNAseq-es that include one or more regression factors. As an example, with reference to FIG. 35, RNA analysis results 3500 may depict RNAseq-es that have a regression factor magnitude greater than or equal to a threshold regression factor. The regression factor magnitude may be denoted by a color as indicated by regression coefficient key 3520. As an example, the threshold regression factor may be 0.3. In this example, only those clusters whose regression factor magnitude is 0.3 or greater may be indicated by RNA analysis result 3500. As seen in FIG. 35, three distinct immune cell parameter groups (e.g., T cell motility and immune phenotype parameters (TMIP)) are present. The three immune cell parameter groups are labeled by (a) the red square. (b) the green square, and (c) three blue squares. These three groups are presented with linearly independent combinations of RNseq clusters from the UMAP clustering. For example, TMIP group (a) may include the stromal immune suppression sub-group, sub-group II. Sub-group II may be associated with tumor stroma growth/decay constant Cs. As another example. TMIP group (b) may include the tumor proliferation sub-group, sub-group III, and the normal stroma sub-group, sub-group IV. Sub-groups III and IV may be associated with tumor epithelium growth/decay constant Ce. As yet another example. TMIP group (c) may include the anti-tumor inflammation sub-group, sub-group I, the normal stroma sub-group IV, and the myelomonocytic immune suppression sub-group, sub-group V. Sub-groups I, IV, and V may be associated with the stroma-amplitude As, the tumor epithelium amplitude Ae, the stroma-immune cell density (Fs), the epithelium-immune cell density (Fe), and/or an epithelium-stroma immune cell density (Mp).

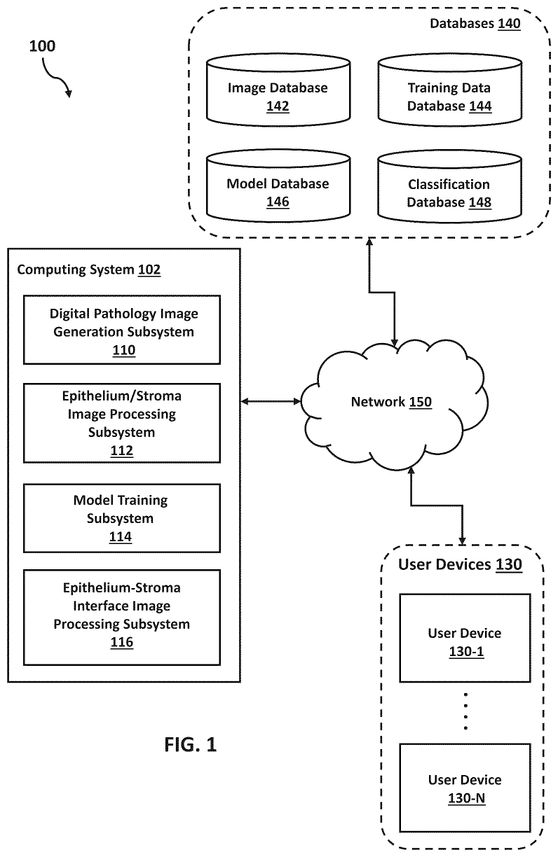

FIG. 1 illustrates an example system 100 for assessing a level of infiltration of immune cells to a tumor, in accordance with various embodiments. System 100 may include a computing system 102, user devices 130 (e.g., user device 130-1 to 130-N), databases 140 (e.g., image database 142, training data database 144, model database 146, classification data database 148), or other components. In some embodiments, components of system 100 may communicate with one another using network 150, such as the Internet.

User devices may be capable of communicating with one or more components of system 100 via network 150 and/or via a direct connection. User device 130 may refer to a computing device configured to interface with various components of system 100 to control one or more tasks, cause one or more actions to be performed, or effectuate other operations. For example, user device 130 may be configured to receive and display an image of a scanned biological sample. Example computing devices that user devices 130 may correspond to include, but are not limited to, which is not to imply that other listings are limiting, desktop computers, servers, mobile computers, smart devices, wearable devices, cloud computing platforms, or other client devices. In some embodiments, each user device 130 may include one or more processors, memory, communications components, display components, audio capture/output devices, image capture components, or other components, or combinations thereof. Each user device 130 may include any type of wearable device, mobile terminal, fixed terminal, or other device.

It should be noted that, while one or more operations are described herein as being performed by particular components of computing system 102, those operations may, in some embodiments, be performed by other components of computing system 102 or other components of system 100. As an example, while one or more operations are described herein as being performed by components of computing system 102, those operations may, in some embodiments, be performed by components user devices 130. It should also be noted that, although some embodiments are described herein with respect to machine learning models, other prediction models (e.g., statistical models or other analytics models) may be used in lieu of or in addition to machine learning models in other embodiments (e.g., a statistical model replacing a machine-learning model and a non-statistical model replacing a non-machine-learning model in one or more embodiments).

Although a single instance of computing system 102 and user device 130 are depicted within system 100, additional instances of one or more of computing system 102 and/or user device 130 may be included. Furthermore, computing system 102 may include a digital pathology image generation subsystem 110, an epithelium/stroma image processing subsystem 112, a model training subsystem 114, an epithelium-stroma interface image processing subsystem 116, or other components. Each of digital pathology image generation subsystem 110, epithelium/stroma image processing subsystem 112, model training subsystem 114, and epithelium-stroma interface image processing subsystem 116 may be configured to communicate with one another, one or more other devices, systems, servers, etc., using one or more communication networks (e.g., the Internet, an Intranet). System 100 may also include one or more databases 140 (e.g., image database 142, training data database 144, model database 146, classification data database 148) used to store data for training machine learning models, storing machine learning models, or storing other data used by one or more components of system 100. This disclosure anticipates the use of one or more of each type of system and component thereof without necessarily deviating from the teachings of this disclosure.

Although not illustrated, other intermediary devices (e.g., data stores of a server connected to Epithelium/stroma image processing subsystem 112 computing system 102) can also be used. Epithelium/stroma image processing subsystem 112

The components of system 100 of FIG. 1 can be used in a variety of contexts where scanning and evaluating digital pathology images, such as whole slide images, are essential components of the work. As an example, system 100 can be associated with a clinical environment where a user is evaluating the sample for possible diagnostic purposes. The user can review the image using user device 130 prior to providing the image to Epithelium/stroma image processing subsystem 112 computing system 102. The user can provide additional information to computing system 102 (e.g., epithelium/stroma image processing subsystem 112, epithelium-stroma interface image processing subsystem 116) that can be used to guide or direct the analysis of the image. For example, the user can provide a prospective diagnosis or preliminary assessment of features within the scan. The user can also provide additional context, such as the type of tissue being reviewed. As another example, system 100 can be associated with a laboratory environment where tissues are being examined, for example, to determine the efficacy or potential side effects of a drug. In this context, it can be commonplace for multiple types of tissues to be submitted for review to determine the effects on the whole body of said drug. This can present a particular challenge to human scan reviewers, who may need to determine the various contexts of the images, which can be highly dependent on the type of tissue being imaged. These contexts can optionally be provided to computing system 102 (e.g., epithelium/stroma image processing subsystem 112, epithelium-stroma interface image processing subsystem 116).

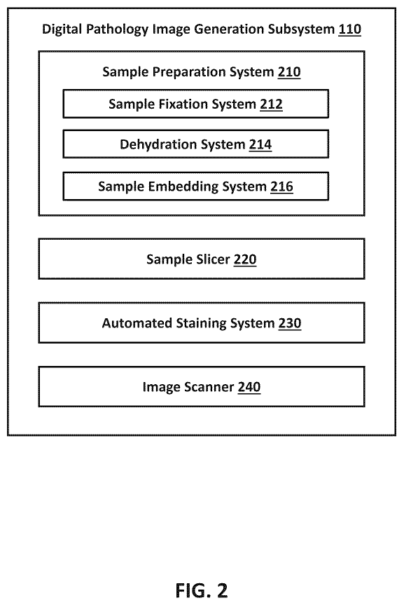

In some embodiments, digital pathology image generation subsystem 110 may be configured to generate one or more whole slide images or other related digital pathology images, corresponding to a particular sample. For example, an image generated by digital pathology image generation subsystem 110 may include a stained section of a biopsy sample. As another example, an image generated by digital pathology image generation subsystem 110 may include a slide image (e.g., a blood film) of a liquid sample. As yet another example, an image generated by digital pathology image generation subsystem 110 can include fluorescence microscopy such as a slide image depicting fluorescence in situ hybridization (FISH) after a fluorescent probe has been bound to a target DNA or RNA sequence. Digital pathology image generation subsystem 110 may include one or more systems, modules, devices, or other components. As an example, with reference to FIG. 2, digital pathology image generation subsystem 110 may include a sample preparation system 210, a sample slicer 220, an automated staining system 230, an image scanner 240, or other components.

Sample preparation system 210 may be configured to prepare a biological sample for digital pathology analyses. Some example types of samples include biopsies, solid samples, samples including tissue, or other biological samples. Sample preparation system 210 may be configured to fix and/or embed a sample. In some embodiments, sample preparation system 210 may facilitate infiltrating a sample with a fixating agent (e.g., liquid fixing agent, such as a formaldehyde solution) and/or embedding substance (e.g., a histological wax). Sample preparation system 210 may include one or more systems, subsystems, modules, or other components, such as a sample fixation system 212, a dehydration system 214, a sample embedding system 216, or other subsystems. Sample fixation system 212 may be configured to fix a biological sample. For example, sample fixation system 212 may expose a sample to a fixating agent for at least a threshold amount of time (e.g., at least 3 hours, at least 6 hours, at least 13 hours, etc.). Dehydration system 214 may be configured to dehydrate the biological sample. For example, dehydration system 214 may expose the fixed sample and/or a portion of the fixed sample to one or more ethanol solutions. In some embodiments, dehydration system 214 may also be configured to clear the dehydrated sample using a clearing intermediate agent. An example clearing intermediate agent may include ethanol and a histological wax. Sample embedding system 216 may be configured to infiltrate the biological sample. In some embodiments, sample embedding system 216 may infiltrate the biological sample using a heated histological wave (e.g., liquid). In some embodiments, sample embedding system 216 may perform the infiltration process one or more times for corresponding predefined time periods. The histological wax can include a paraffin wax and potentially one or more resins (e.g., styrene or polyethylene). Sample preparation system 210 may further be configured to cool the biological sample and wax or otherwise allow the biological sample and wax to be cooled. After cooling. Sample preparation system 210 may block out the wax-infiltrated biological sample.

Sample slicer 220 may be configured to receive the fixed and embedded sample and produce a set of sections. Sample slicer 220 can expose the fixed and embedded sample to cool or cold temperatures. Sample slicer 220 can then cut the chilled sample (or a trimmed version thereof) to produce a set of sections. For example, each section may have a thickness that is less than 100 μm, less than 50 μm, less than 10 μm, less than 5 μm, or other dimensions. As another example, each section may have a thickness that is greater than 0.1 μm, greater than 1 μm, greater than 2 μm, greater than 4 μm, or other dimensions. The sections may have the same or similar thickness as the other sections. For example, a thickness of each section may be within a threshold tolerance (e.g., less than 1 μm, less than 0.1 μm, less than 0.01 μm, or other values). The cutting of the chilled sample can be performed in a warm water bath (e.g., at a temperature of at least 30° C., at least 35° C., at least 40° C., or other temperatures).

Automated staining system 230) may be configured to stain one or more of the sample sections. Automated staining system 230 may expose each section to one or more staining agents. Example staining agents may include Hematoxylin, Eosin, PanCK, and CD8. In one example, a panCK-CD8 dual-stain may be used as the staining agent. As an example, with reference to FIG. 26, image 2600 represents a whole slide image, or a portion of a whole slide image, depicting a biological sample that has been stained using a panCK-CD8 dual stain. In image 2600, certain biological structures (e.g., immune cells) may be represented by the “brown” spots highlighted from the CD8 staining agent, while other biological structures (e.g., tumor epithelium) may be represented by the “purple” spots highlighted from the panCK staining agent. Each section can be exposed to a predefined volume of a staining agent for a predefined period of time. In some embodiments, automated staining system 230 may be configured to concurrently or sequentially expose a single section to multiple staining agents.

Each of one or more stained sections can be presented to image scanner 240, which can capture a digital image of that section. Image scanner 240) can include a microscope camera. Image scanner 240 may be configured to capture the digital image at one or more levels of magnification (e.g., using a 10× objective, a 20× objective, a 40× objective, or other magnification levels). Manipulation of the image can be used to capture a selected portion of the sample at the desired range of magnifications. Image scanner 240 can further capture annotations and/or morphometrics identified by a human operator. In some embodiments, a section may be returned to automated staining system 230 after one or more images are captured, such that the section can be washed, exposed to one or more other stains, and imaged again. In some embodiments, when multiple stains are used, these stains can be selected to have different color profiles. For example, a first region of an image corresponding to a first section that absorbed a large amount of a first staining agent can be distinguished from a second region of the image (or a different image) corresponding to a second section that absorbed a large amount of a second staining agent.

It will be appreciated that one or more components of digital pathology image generation subsystem 110 can, in some instances, operate in connection with human operators. For example, human operators can move the sample across various components of digital pathology image generation subsystem 110 Epithelium/stroma image processing subsystem 112 and/or initiate or terminate operations of one or more subsystems, systems, or components of digital pathology image generation subsystem 110. As another example, part or all of one or more components of the digital pathology image generation system (e.g., one or more subsystems of sample preparation system 210) can be partly or entirely replaced with actions of a human operator.

Further, it will be appreciated that, while various described and depicted functions and components of digital pathology image generation subsystem 110 pertain to processing of a solid and/or biopsy sample, other embodiments can relate to a liquid sample (e.g., a blood sample). For example, digital pathology image generation subsystem 110 can receive a liquid-sample (e.g., blood or urine) slide that includes a base slide, smeared liquid sample, and a cover. In some embodiments, image scanner 240 may capture an image of the sample slide. Furthermore, some embodiments of digital pathology image generation subsystem 110 include capturing images of samples using advancing imaging techniques, such as FISH, described herein. For example, after a fluorescent probe has been introduced to a sample and allowed to bind to a target sequence, appropriate imaging techniques can be used to capture images of the sample for further analysis.

A given sample can be associated with one or more users (e.g., one or more physicians, laboratory technicians and/or medical providers) during processing and imaging. An associated user can include, by way of example and not of limitation, a person who ordered a test or biopsy that produced a sample being imaged, a person with permission to receive results of a test or biopsy, or a person who conducted analysis of the test or biopsy sample, among others. For example, a user can correspond to a physician, a pathologist, a clinician, or a subject. A user can use one or more user devices 130 to submit one or more requests (e.g., that identify a subject) that a sample be processed by digital pathology image generation subsystem 110 and that a resulting image be processed by epithelium/stroma image processing subsystem 112 and/or epithelium-stroma interface image processing subsystem 116, or another component of system 100.

In some embodiments, the biological samples that will be prepared for imaging by image scanner 240 may include images collected from one or more clinical trials. In one example, the clinical trials may include an NSCLC clinical trial, which may include biological samples of adenocarcinomas and squamous cell carcinoma.

Digital pathology image generation subsystem 110 may be configured to transmit an image produced by image scanner 240 to user device 130. User device 130 may communicate with epithelium/stroma image processing subsystem 112 and/or epithelium-stroma interface image processing subsystem 116 to initiate automated processing of an image. In some embodiments, digital pathology image generation subsystem 110 may be configured to provide an image produced by image scanner 240 to epithelium/stroma image processing subsystem 112 and/or epithelium-stroma interface image processing subsystem 116 Epithelium/stroma image processing subsystem 112. For example, an image may be directed from image scanner 240 to epithelium/stroma image processing subsystem 112 and/or epithelium-stroma interface image processing subsystem 116 by a user of user device 130. In some embodiments, the clinical trials may evaluate an effectiveness of an immunotherapy in treating an underlying condition (e.g., NSCLC). For example, the immunotherapy being evaluated may be atezolizumab, docetaxel, or another immunotherapy. The number of patients involved in a clinical trial may vary. For example, the number of patients may be 100 or more, 250 or more, 1,000 or more, and the like.

In some embodiments, patient response to immunotherapies may be predicted based on immune cell distributions of tumor samples. Characteristics of the immune cell distribution may be identified using a digital pathology. Digital pathology can potentially provide more information than bulk RNAseq or manual immune cell (e.g., T cell) density evaluation because it allows a highly quantitative positional analysis of individual immune cells in the sample. If immune cells are absent or unable to reach their target (e.g., tumor cells), immunotherapy may fail. Therefore, it follows that immune cells need to be present to attack and kill tumor cells.

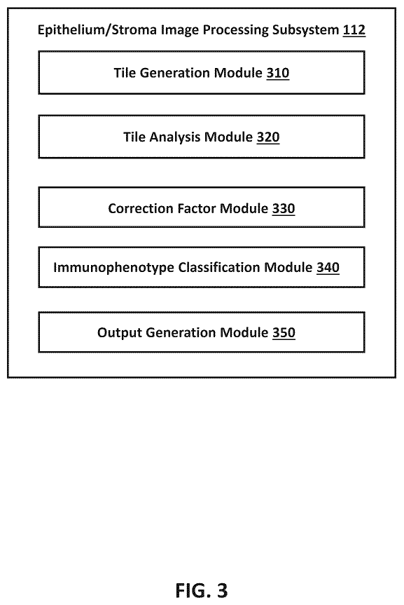

Epithelium/stroma image processing subsystem 112 may be configured to process digital pathology images (e.g., whole slide images). Epithelium/stroma image processing subsystem 112 may be configured to classify the digital pathology images and generate annotations for these digital pathology images and related output. As an example, with reference to FIG. 3, epithelium/stroma image processing subsystem 112 may include one or more subsystems, such as a tile generation module 310, a tile analysis module 320, a correction factor module 330, an immunophenotype classification module 340, an output generation module 350, or other components.

Tile generation module 310 may be configured to generate a set of tiles for each digital pathology image. Digital pathology can potentially provide more information than bulk RNAseq or manual T cell density evaluation because it allows the high quantitative positional analysis of individual immune cells in the sample. If T cells are absent or unable to reach their target, immunotherapy may fail. It stands to reason that the T cells need to be present to kill tumor cells. Epithelium/stroma image processing subsystem 112 may further be configured to manage requests to access whole slide images from other components of system 100, including user device 130 and/or image database 142. For example, epithelium/stroma image processing subsystem 112 may receive requests to identify a whole slide image based on a particular tile, an identifier for the tile, or an identifier for the whole slide image. Epithelium/stroma image processing subsystem 112 can perform tasks such as: confirming that the whole slide image is available to the requesting user, identifying the appropriate databases from which to retrieve the requested whole slide image (e.g., image database 142), and retrieving any metadata that may be of interest to the requesting user or module. Additionally, epithelium/stroma image processing subsystem 112 can efficiently handle streaming the appropriate data to the requesting device.

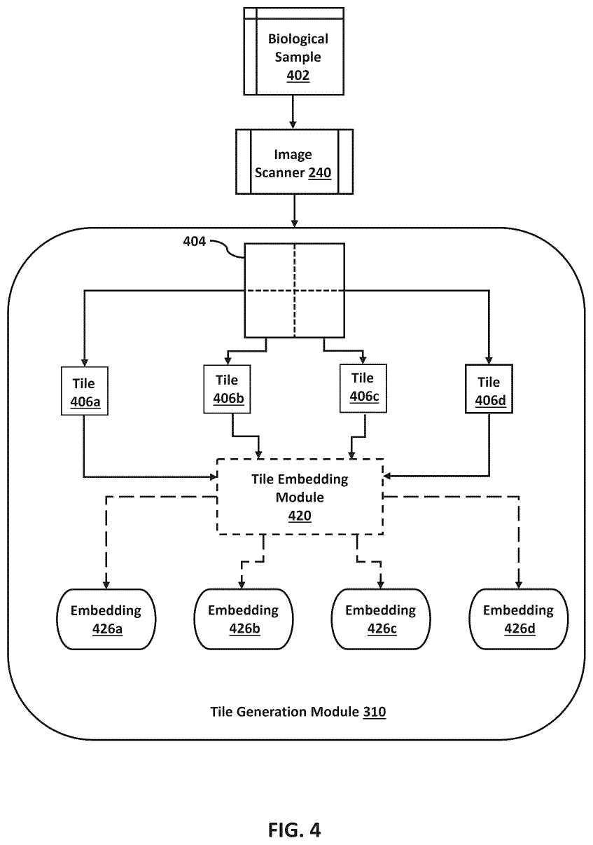

As an example, with reference to FIG. 4, tile generation module 310 may receive a digital pathology image 404 of a biological sample 402 that has been imaged by image scanner 240) (previously described with reference to FIG. 2). Tile generation module 310 may segment digital pathology image 404 into a set of tiles 406a-406d (collectively “tiles 406”). In some embodiments, tiles 406 can be non-overlapping (e.g., each tile includes pixels of digital pathology image 404 not included in any other tile) or overlapping (e.g., each tile includes some portion of pixels of digital pathology image 404 that are included in at least one other tile). Features such as whether tiles 406 overlap, in addition to a size of each tile and the stride window (e.g., the image distance or number of pixels between a tile and a subsequent tile) can increase or decrease the data set for analysis, with more tiles (e.g., through overlapping or smaller tiles) increasing the potential resolution of eventual outputs and visualizations. In some embodiments, tile generation module 310 may define a set of tiles for an image where each tile is of a predefined size and/or an offset between tiles is predefined. Furthermore, tile generation module 310 may generate multiple sets of tiles of varying size, overlap, step size, etc., for each digital pathology image. In some embodiments, digital pathology image 404 itself can contain tile overlap, which may result from the imaging technique. In some embodiments, tile segmentation without overlapping tiles can balance tile processing requirements and can avoid influencing the embedding generation and weighting value generation. A tile size or tile offset can be determined, for example, by calculating one or more performance metrics (e.g., precision, recall, accuracy, and/or error) for each size/offset and by selecting a tile size and/or offset associated with one or more performance metrics above a predetermined threshold and/or associated with one or more optimal (e.g., high precision, highest recall, highest accuracy, and/or lowest error) performance metric(s).

Tile generation module 310 may further be configured to define a tile size. The tile size may be determined based on a type of abnormality being detected. For example, tile generation module 310 may be configured to set the tile size for segmentation of digital pathology image 404 based on the types of tissue abnormalities present in biological sample 402. Tile generation module 310 may also customize the tile size based on the tissue abnormalities to be detected/searched for to optimize detection. In some embodiments, tile generation module 310 may determine that, when the tissue abnormalities include inflammation or necrosis in lung tissue, the tile size should be reduced to increase the scanning rate. In some embodiments, tile generation module 310 may determine that, when the tissue abnormalities include abnormalities with Kupffer cells in liver tissues, the tile size should be increased to increase the opportunities for epithelium/stroma image processing subsystem 112 to analyze the Kupffer cells holistically. In some embodiments, tile generation module 310 may define a set of tiles where a number of tiles in the set, a size of the tiles of the set, a resolution of the tiles for the set, or other related properties, for each image may be defined and held constant for each of one or more images.

In some embodiments, tile generation module 310 may be configured to receive digital pathology image 404 of biological sample 402 (e.g., a tumor). Digital pathology image 404 of biological sample 402 may include a whole slide image (WSI). For example, as mentioned above, digital pathology image generation subsystem 110 may be configured to produce a WSI of a biological sample (e.g., a tumor). In some embodiments, digital pathology image generation subsystem 110 may generate multiple digital pathology images of a biological sample at different settings. For example, image scanner 240 may capture images of biological sample 402 at multiple magnification levels (e.g., 5×, 10×, 20×, 40×, etc.). These images may be provided to epithelium/stroma image processing subsystem 112 as a stack, and an operator may determine which image or images from the stack to be used for the subsequent analysis. The biological sample may be prepared, sliced, stained, and subsequently imaged to produce the WSI. The biological sample may include a biopsy of a tumor. For example, the biological sample may include tumors from NSCLC clinical trials.

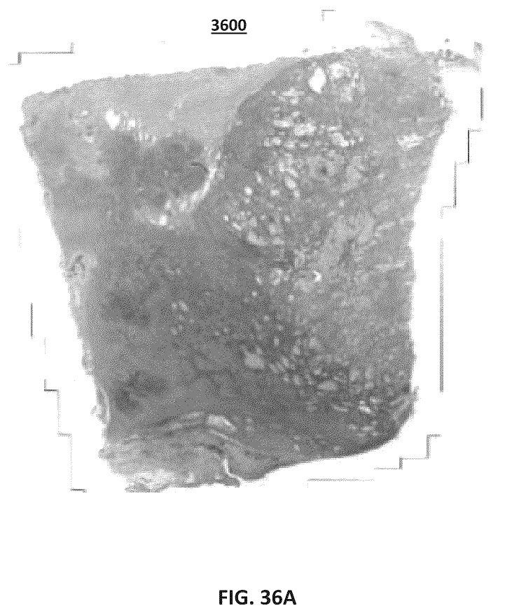

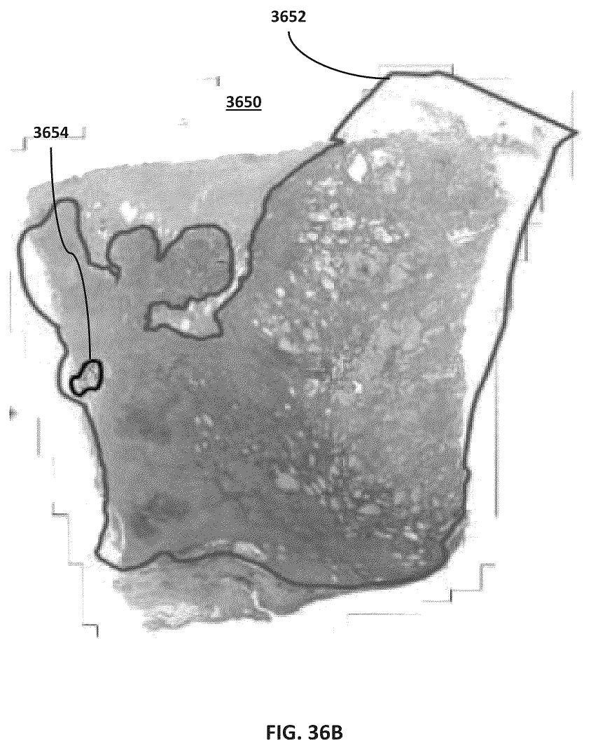

In some embodiments, a region of interest of digital pathology image 404 may be identified prior to tiling. For example, a pathologist may manually define the region of interest (ROI) in the tumor. The ROI may be defined using a digital pathology image viewing system at a particular magnification (e.g., 4x). As another example, a machine learning model may be used to define the ROI in the tumor lesion. In this example, a human (e.g., pathologist) may be able to review the machine-defined ROI (e.g., to confirm that the defined ROI is accurate). The defined ROI may exclude areas of necrosis. This is important because some staining agents can label normal epithelial cells, not just tumor epithelium. As an example, with reference to FIGS. 36A-36B, whole slide image 3600 may depict a biological sample, such as a tumor including tumor epithelium and surrounding tumor stroma, and whole slide image 3650 may include a region of interest (ROI) 3652. In some embodiments, ROI 3652 may correspond to a portion of the biological sample depicting the tumor, excluding other portions of the biological sample (e.g., areas of necrosis). In some embodiments, whole slide image 3650 may also depict a region of interest 3654. In some embodiments, the version of digital pathology image 404 that is tiled (e.g., producing tiles 406) corresponds to region of interest 3652 and/or 3654. Therefore, the total size of digital pathology image 404 that will be tiled can be smaller than that of the original image.

In some embodiments, a color deconvolution may be performed to tiles 406. The color deconvolution may separate out each color channel from the image tile, obtaining a version of the image tile for each color channel. Alternatively, color deconvolution may be performed at the whole slide image level. In this example, digital pathology image 404 may be deconvolved into a plurality of color channels (e.g., three color channels), and tiles 406 can be produced for each color channel. Different staining agents may cause biological sample 402 to turn different colors. Furthermore, certain staining agents may interact with specific portions of biological sample 402. For example, one staining agent (e.g., CD8) may cause immune cells to turn one color, while another staining agent (e.g., panCK) may cause tumor stroma to turn another color.

In some embodiments, digital pathology images received by epithelium/stroma image processing subsystem 112 can include large-format multi-color channel images having pixel color values for each pixel of the image specified for one of several color channels. Example color specifications or color spaces that can be used include the RGB, CMYK, HSL, HSV, or HSB color specifications. Tiles 406 can be defined based on segmenting the color channels and/or generating a brightness map or grayscale equivalent for each tile. For example, for each (multi-color channel) tile, a red tile, a blue tile, a green tile, and/or a brightness tile, or the equivalent for the color specification used, may be provided. As explained herein, segmenting the digital pathology images based on segments of the image and/or color values of the segments can improve the accuracy and recognition rates of the networks used to generate embeddings for the tiles and image and to produce classifications of the image. Additionally, tile generation module 310 may be configured to convert between color specifications and/or prepare copies of the tiles using multiple color specifications. Color specification conversions can be selected based on a desired type of image augmentation (e.g., accentuating or boosting particular color channels, saturation levels, brightness levels, etc.). Color specification conversions can also be selected to improve compatibility between digital pathology image generation subsystem 110 and epithelium/stroma image processing subsystem 112. For example, a particular image scanning component can provide output in the HSL color specification, and the models used by epithelium/stroma image processing subsystem 112 can be trained using RGB images. Converting tiles 406 to the compatible color specification can ensure that tiles 406 can still be analyzed. Additionally, epithelium/stroma image processing subsystem 112 can up-sample or down-sample images that are provided in particular color depth (e.g., 8-bit, 16-bit, etc.) to be usable by epithelium/stroma image processing subsystem 112. Furthermore, epithelium/stroma image processing subsystem 112 can cause tiles 406 to be converted according to the type of image that has been captured (e.g., fluorescent images may include greater detail on color intensity or a wider range of colors).

In some embodiments, the color deconvolution process may be used to generate a clustering map 2710, as seen in FIG. 27. Clustering map 2710 may include a red outline defining a region of a tumor, the tumor including areas of tumor epithelium and areas of surrounding tumor stroma. In clustering map 2710, different biological structures may be highlighted using different staining agents and may be depicted by different colored data points. For example, tumor epithelium cells may be depicted by “blue” data points, while tumor stroma cells may be depicted by “turquoise” data points. Clustering map 2710 may also indicate an amount of area occupied by each of the biological structures. For example, the areas of tumor epithelium may comprise approximately 31% of the tumor depicted, while the areas of tumor stroma may comprise approximately 50% of the tumor depicted.

In some embodiments, artifacts from tiles 406 may be corrected. For example, artifacts may be present in a tile where the tumor epithelial tissue shrank away from the tumor stromal areas. A correction factor may be used to remove these artifacts from the image tile. Removal of the artifacts can reduce the likelihood of the artifacts impacting downstream classification tasks of the digital pathology analysis pipeline.

In some embodiments, tiles 406 including tumor epithelium and tumor stroma may be identified. Tiles 406 including tumor epithelium and tumor stroma may be identified using computer vision models (e.g., convolutional neural networks), by a trained pathologist, or a combination thereof. For example, a computer vision model may identify tiles 406 including tumor epithelium and tumor stroma, and the trained pathologist can confirm the selection and/or edit the selection. In some embodiments, at least one of tiles 406 may include a unique portion of the image. In some embodiments, a random subset or pseudo-random subset of tiles 406 that include tumor epithelium and tumor stroma may be selected.

In some embodiments, tile generation module 310 may also include a tile embedding module 420. Tile embedding module 420 may receive tiles 406 and generate embeddings 426a-426d (collectively “embeddings 426”). In some embodiments, tile embedding module 420 may be configured to generate an embedding for each tile in a corresponding feature embedding space. Embedding 426a-426d can be represented by epithelium/stroma image processing subsystem 112 as a feature vector for the tile. Tile embedding module 420 may implement a neural network (e.g., a convolutional neural network, residual neural network, etc.). For each of tiles 406, the neural network implemented by tile embedding module 420) may be trained to generate an embedding representing that tile in a multi-dimensional feature space. In some embodiments, the neural network can be based on the ResNet image network. The neural network may be trained on a dataset of medical images and/or a dataset based on natural (e.g., non-medical) images, such as the ImageNet dataset. By using a non-specialized tile embedding network, tile embedding module 420 can leverage known advances in efficiently processing images to generate embeddings. Furthermore, using a natural image dataset allows the neural network to learn to discern differences between tiles on a holistic level.

In other embodiments, the neural network used by tile embedding module 420) can be an embedding network customized to handle large numbers of tiles of large format images, such as tiles derived from digital pathology whole slide images. Additionally, the neural network used by tile embedding module 420 can be trained using custom training data.

For example, the tile embedding neural network can be trained using a variety of samples of whole slide images or even trained using samples relevant to the subject matter for which the embedding network will be generating embeddings (e.g., scans of particular tissue types). Training the tile embedding neural network using specialized or customized sets of images can allow the tile embedding neural network to identify fine differences between tiles which can result in more detailed and accurate distances between tiles in the feature embedding space at the cost of additional time to acquire the images and the computational and economic cost of training multiple tile-generating networks for use by tile embedding module 420. Tile embedding module 420 can select from a library of tile embedding networks based on the type of images being processed by epithelium/stroma image processing subsystem 112.

As described herein, embeddings 426 can be generated from a deep learning neural network using visual features of tiles 406. Embeddings 426 can be further generated from contextual information associated with tiles 406 or from the content shown in tiles 406. For example, a tile embedding can include one or more features that indicate and/or correspond to a size of depicted objects (e.g., sizes of depicted cells or aberrations) and/or density of depicted objects (e.g., a density of depicted cells or aberrations). Size and density can be measured absolutely (e.g., width expressed in pixels or converted from pixels to nanometers) or relative to other tiles from the same digital pathology image, from a class of digital pathology images, or from a related family of digital pathology images. Furthermore, tiles 406 can be classified prior to tile embedding module 420 generating embeddings 426 such that tile embedding module 420 considers the pre-determined classifications when preparing embeddings 426.

In some embodiments, tile embedding module 420 may be configured to produce embeddings 426 of a predefined size (e.g., vectors of 512 elements, vectors of 2048 bytes, etc.). This can increase consistency across tiles when being analyzed for downstream classifications. In some embodiments, tile embedding module 420 may be configured to produce embeddings 426 of various and arbitrary sizes. Some embodiments include tile embedding module 420 being configured to adjust the sizes of embeddings 426 based on user direction or can be selected, for example, to optimize computation efficiency, accuracy, or other parameters. In some embodiments, the embedding size can be selected based on the limitations or specifications of the machine learning model (e.g., a CNN) that generated embeddings 426. Larger embedding sizes can be used to increase the amount of information captured in embedding 426 and improve the quality and accuracy of results, while smaller embedding sizes can be used to improve computational efficiency.

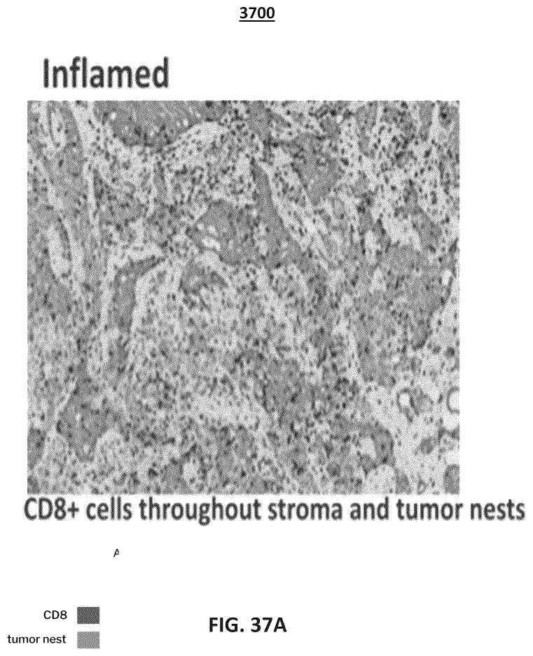

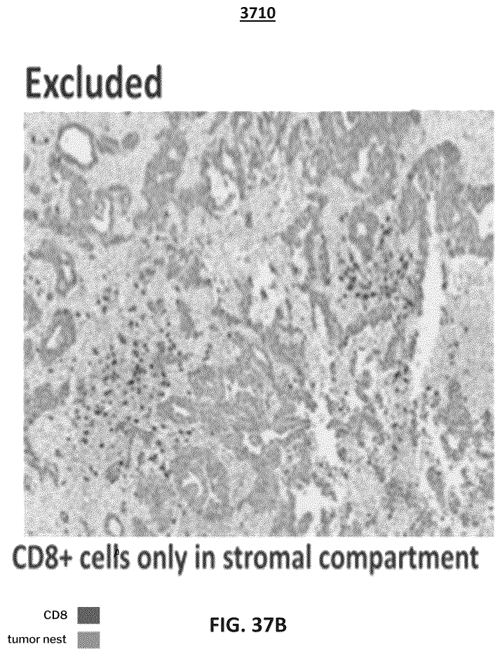

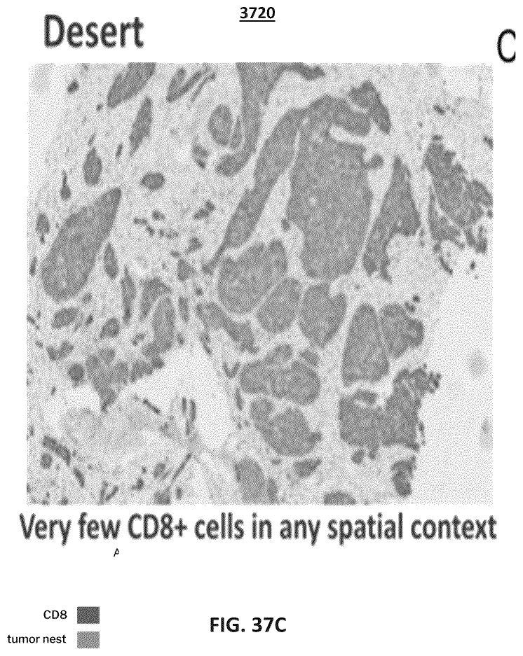

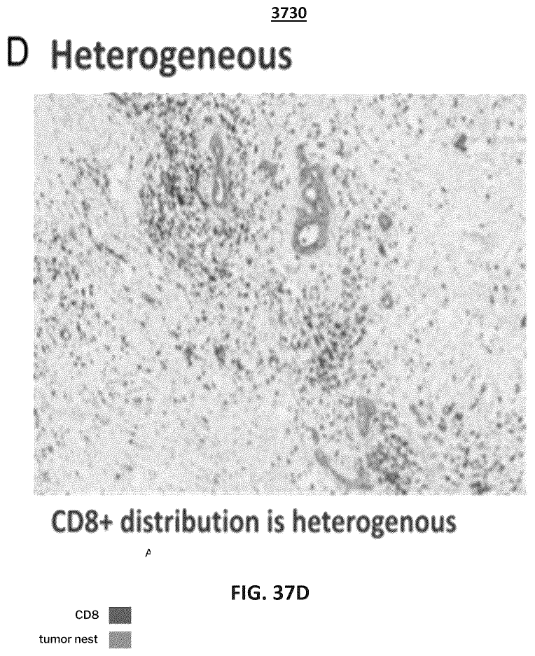

In some embodiments, tile analysis module 320 may be configured to analyze tiles 406 and/or embeddings 426 representing tiles 406 to determine immune cell behavior within a biological sample. As an example, with reference to FIG. 5, tile analysis module 320) may receive tiles 406 and/or embeddings 426 representing tiles 406. Although a single tile 406 and embedding 426 are depicted in FIG. 5, multiple tiles and/or embeddings may be input to tile analysis module 320 sequentially or in parallel. Tiles 406 and/or embeddings 426 selected may include tiles that depict tumor epithelium and tumor stroma. Traditionally, identifying immune cells (e.g., CD8+ T cells) and computing immune cell density occurs separately for tumor epithelium and tumor stroma. The densities were then averaged across each compartment. A high immune cell presence in both the tumor epithelium and the tumor stroma can indicate immune cell infiltration. In this example, the tumor immunophenotype may be classified as “inflamed” or “infiltrated.” For example, tile 3700 of FIG. 37A illustrates an example image tile depicting the “inflamed” tumor immunophenotype classification. In this classification, as seen from image tile 3700, immune cells—highlighted in brown—are distributed throughout the tumor stroma and the tumor epithelium—highlighted in pink. High infiltration in only the tumor stroma but not the tumor epithelium can correspond to a tumor immunophenotype classification of “excluded.” For example, with reference to FIG. 37B, image tile 3710 may include immune cells distributed only in the tumor stroma. Few immune cells found in the tumor stroma and the tumor epithelium can correspond to a tumor immunophenotype classification of “desert.” For example, with reference to FIG. 37C, image tile 3720 may include few immune cells in the tumor stroma or the tumor epithelium. As a comparison, image tile 3730 of FIG. 37D depicts a heterogeneous distribution of immune cells in the tumor stroma and the tumor epithelium.

In some embodiments, tile analysis module 320 may be configured to identify a boundary of a tumor in a digital pathology image. In some embodiments, tile analysis module 320 may use one or more machine learning models to identify tumors within digital pathology images. The machine learning models may include computer vision models trained to recognize objects (e.g., tumor lesions) within an image of a biological sample. In some embodiments, the machine learning models may identify a portion or portions of the image including tumor lesions. The portions of the image forming the tumor lesions may be extracted. In some embodiments, the image tiles forming the image may be analyzed to determine which tiles depict a boundary of the tumor. The tiles associated with the boundary may be extracted. These tiles may form an image to be analyzed for immunophenotyping.

Tile analysis module 320 may include an epithelium-immune cell density calculation module 510) and a stroma-immune cell density calculation module 520. Epithelium-immune cell density calculation module 510 and stroma-immune cell density calculation module 520 may be configured to compute a density of immune cells in the tumor epithelium and the tumor stroma, respectively. In some embodiments, epithelium-immune cell density calculation module 510 and stroma-immune cell density calculation module 520 may compute a density of immune cells in the tumor epithelium and the tumor stroma on a tile-by-tile basis.

Epithelium-immune cell density calculation module 510 may be configured to determine an epithelium-immune cell density 512. Epithelium-immune cell density 512 refers to a density of immune cells in the tumor epithelium (i.e., or the portion of the tumor included in the image tile being analyzed). Epithelium-immune cell density calculation module 510) may be configured to determine epithelium-immune cell density 512 based on a number of immune cells identified in the tumor epithelium depicted by an image tile. For example, epithelium-immune cell density calculation module 510 may determine a number of immune cells in the tumor epithelium depicted by tiles 406. In some embodiments, epithelium-immune cell density calculation module 510 may determine the number of immune cells in the tumor epithelium based on an image tile (e.g., one of tiles 406) and/or an embedding (e.g., one of embeddings 426) associated with that image tile. The types of immune cells that may be found in the tumor epithelium may include T cells, B cells, dendritic cells, macrophages, fibroblasts, hepatocytes, or other immune cells, or combinations thereof.

Stroma-immune cell density calculation module 520 may be configured to determine a stroma-immune cell density 522. Stroma-immune cell density 522 refers to a density of immune cells in the tumor stroma. Stroma-immune cell density calculation module 520) may be configured to determine stroma-immune cell density 522 based on a number of immune cells identified in the tumor stroma depicted by an image tile. For example, stroma-immune cell density calculation module 520 may determine a number of immune cells in the stroma depicted by tiles 406. In some embodiments, stroma-immune cell density calculation module 520 may determine the number of immune cells in the tumor stroma based on an image tile (e.g., one of tiles 406) and/or an embedding (e.g., one of embeddings 426) associated with that image tile. The types of immune cells that may be found in the tumor stroma may include T cells, B cells, dendritic cells, macrophages, fibroblasts, hepatocytes, or other immune cells, or combinations thereof.

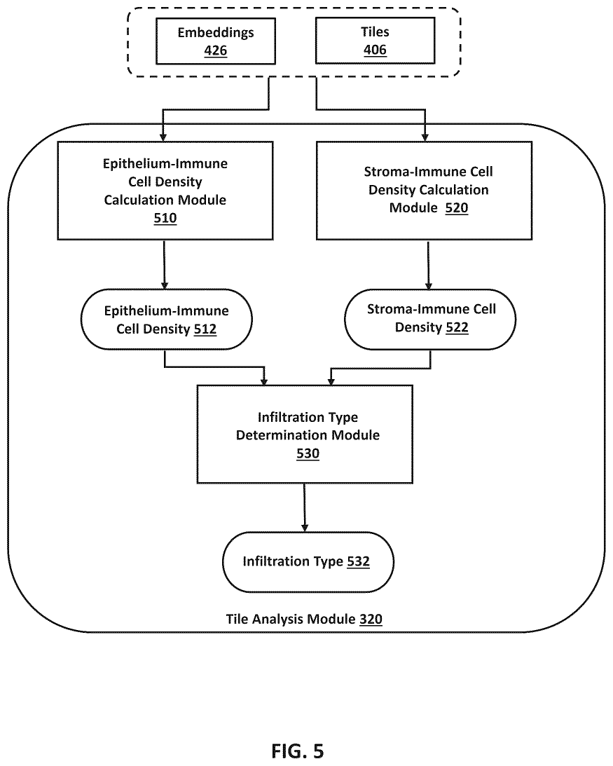

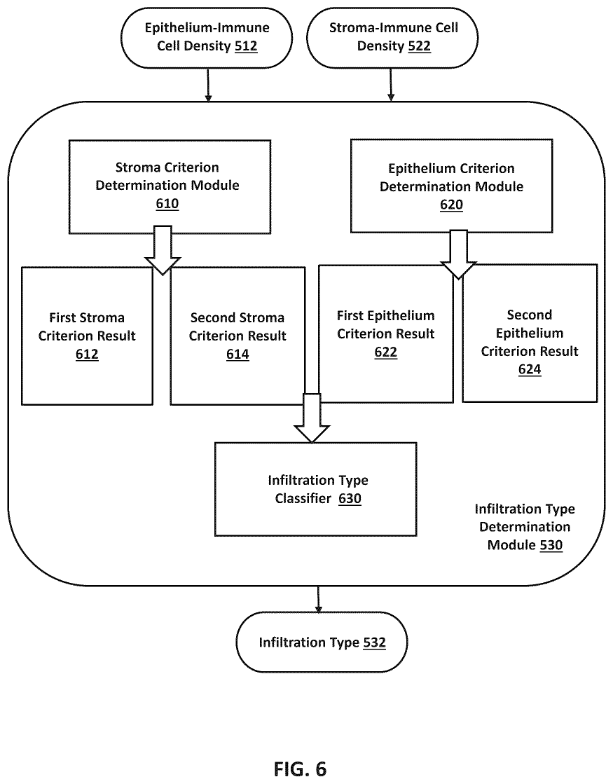

In some embodiments, epithelium-immune cell density 512 and stroma-immune cell density 522 may be provided to an inflammation type determination module 530. Inflammation type determination module 530 may be configured to determine an inflammation type 532 of tiles 406 and/or embeddings 426 based on epithelium-immune cell density 512 and stroma-immune cell density 522. In some embodiments, inflammation type 532 may a first inflammation type or a second inflammation type. Additional details regarding inflammation type determination module 530 is described with reference, for example, to FIG. 6. As seen in FIG. 6, inflammation type determination module 530 may include a stroma criterion determination module 610, an epithelium criterion determination module 620, and an infiltration type classifier 630. As seen above, epithelium-immune cell density 512 and stroma-immune cell density 522 may be input to inflammation type determination module 530.

In some embodiments, stroma criterion determination module 610 may be configured to output a first stroma criterion result 612 for stroma-immune cell density 522. First stroma criterion result 612 may indicate whether a first stroma criterion for stroma-immune cell density 522 has been met. The first stroma criterion being met may include a determination that stroma-immune cell density 522 is greater than or equal to a stroma-immune cell density threshold. As an example, the stroma-immune cell density threshold may be determined based on a number of immune cells determined to be at the ESI. As another example, the stroma-immune cell density threshold may be determined based on a number of immune cells determined to be at the ESI divided by a total number of image tiles. As still another example, the stroma-immune cell density threshold may be determined based on a distribution of the immune cells. The distribution of the immune cells, as described in greater detail below, may be computed based on distance measurements, where the distance measurements represent a distance from immune cell nuclei to the ESI.

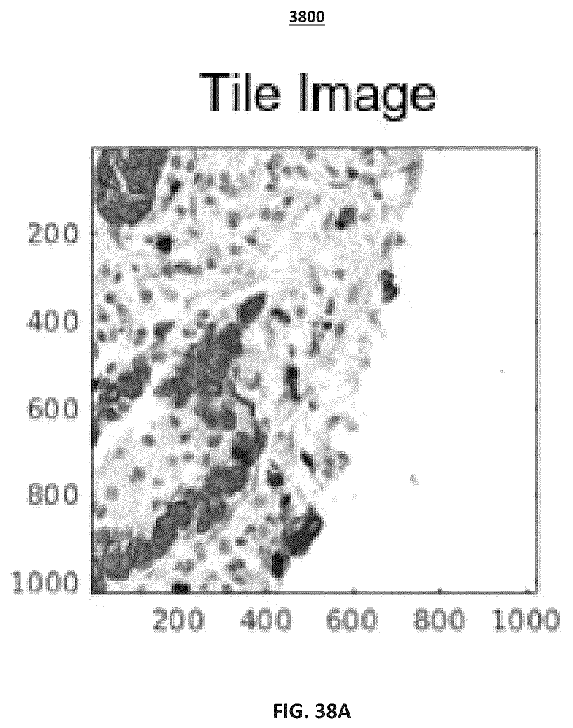

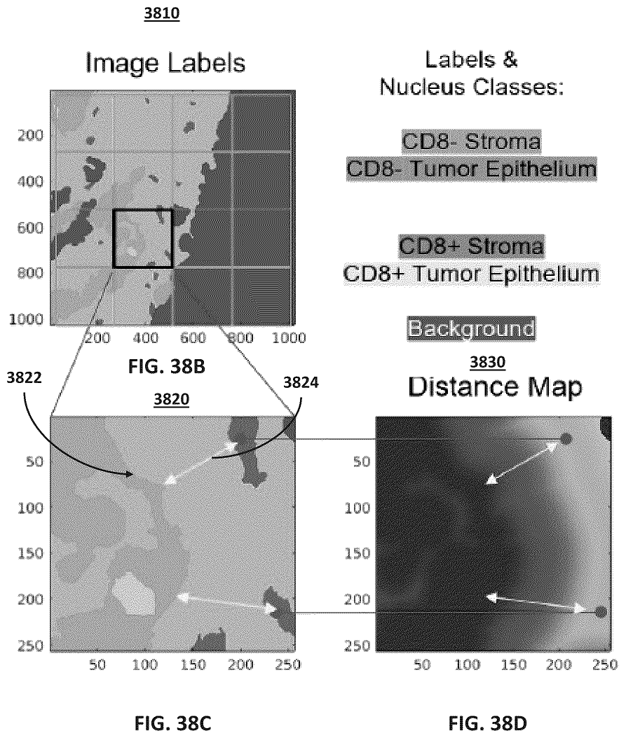

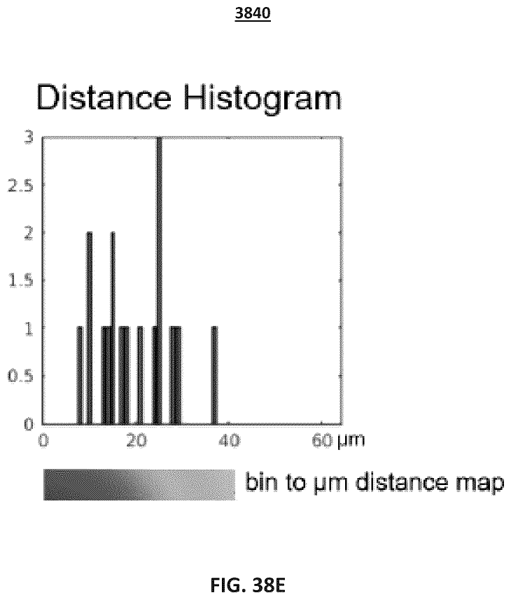

As an example, with reference to FIG. 38A, image 3800 may depict a region of a tumor tissue sample. The tissue sample may have had one or more staining agents applied thereto, which can cause different cellular structures to be highlighted in different colors. FIG. 38B illustrates image tiles 3810 including image labels indicating the various cellular structures identified by application of different staining agents. Furthermore, image tiles 3810 may denote the image tiles obtained by splitting image 3800 into tiles. FIG. 38C illustrates an example image tile 3820 selected from image tiles 3810. In some embodiments, image tile 3820 may be selected based on its inclusion of tumor stroma and tumor epithelium. Image tile 3820 may be analyzed to determine a distance 3824 of CD8+ nuclei to ESI 3822 indicating a boundary of the tumor epithelium from the tumor stroma. For example, distance 3824 may represent a distance from a CD8+ nuclei, represented by the black dot within CD8+ stroma in red, to a stroma-epithelium interface (e.g., the interface of the CD8− stroma to the CD8− tumor epithelium). FIG. 38D illustrates distance map 3830 describing a relationship between the identified cellular structures from image tile 3820. FIG. 38E illustrates a distance histogram 3840.

In some embodiments, stroma criterion determination module 610 may be configured output a second stroma criterion result 614 for epithelium-immune cell density 512. Second stroma criterion result 614 may indicate whether a second stroma criterion for stroma-immune cell density 522 has been met. The second stroma criterion being met may include epithelium-immune cell density 512 being greater than or equal to an epithelium-immune cell density threshold. As an example, the epithelium-immune cell density threshold may be determined based on a number of immune cells determined to be at the ESI. As another example, the stroma-immune cell density threshold may be determined based on a number of immune cells determined to be at the ESI divided by a total number of image tiles. As still another example, the stroma-immune cell density threshold may alternatively be determined based on a distribution of the immune cells. The distribution of the immune cells, as described in greater detail below, may be computed based on distance measurements, where the distance measurements represent a distance from immune cell nuclei to the ESI.

In some embodiments, epithelium criterion determination module 620 may be configured to output a first epithelium criterion result 622 for stroma-immune cell density. First epithelium criterion result 622 may indicate whether a first epithelium criterion for stroma-immune cell density 522 has been met. The first epithelium criterion being met may include stroma-immune cell density 522 being less than or equal to the stroma-immune cell density threshold. As an example, the stroma-immune cell density threshold may be determined based on a number of immune cells determined to be at the ESI. As another example, the stroma-immune cell density threshold may be determined based on a number of immune cells determined to be at the ESI divided by a total number of image tiles. As still another example, the stroma-immune cell density threshold may alternatively be determined based on a distribution of the immune cells. The distribution of the immune cells, as described in greater detail below, may be computed based on distance measurements, where the distance measurements represent a distance from immune cell nuclei to the ESI.

In some embodiments, epithelium criterion determination module 620 may be configured to output a second epithelium criterion result 624 for epithelium-immune cell density 512. Second epithelium criterion result 624 may indicate whether a second epithelium criterion for epithelium-immune cell density 512 has been met. The second epithelium criterion for epithelium-immune cell density 512 being met may include epithelium-immune cell density 512 being less than or equal to the epithelium-immune cell density threshold. As an example, the epithelium-immune cell density threshold may be determined based on a number of immune cells determined to be at the ESI. As another example, the stroma-immune cell density threshold may be determined based on a number of immune cells determined to be at the ESI divided by a total number of image tiles. As still another example, the stroma-immune cell density threshold may alternatively be determined based on a distribution of the immune cells. The distribution of the immune cells, as described in greater detail below, may be computed based on distance measurements, where the distance measurements represent a distance from immune cell nuclei to the ESI.

In some embodiments, first stroma criterion result 612 and second stroma criterion result 614 may be provided to infiltration type classifier 630. Additionally, first epithelium criterion result 622 and second epithelium criterion result 624 may be provided to infiltration type classifier 630. In some embodiments, infiltration type classifier 630 may be configured to classify the tumor region depicted by the input image tile (e.g., the image tile represented by embedding 426) into one or more inflammation types. For example, the inflammation types may include a first inflammation type and a second inflammation type. Infiltration type classifier 630 may be configured to determine whether the inflammation type of the tumor depicted by each of tiles 406 is of the first inflammation type or the second inflammation type. In some embodiments, infiltration type classifier 630 may classify the image (e.g., digital pathology image 404) as being the first inflammation type based on (i) the first stroma criterion for stroma-immune cell density 522 being met and (ii) the second stroma criterion for epithelium-immune cell density 512 being met. In some embodiments, infiltration type classifier 630 may classify the image (e.g., digital pathology image 404) as being of the second inflammation type based on (iii) the first epithelium criterion for stroma-immune cell density 522 being met and (iv) the second epithelium criterion for epithelium-immune cell density 512 being met.

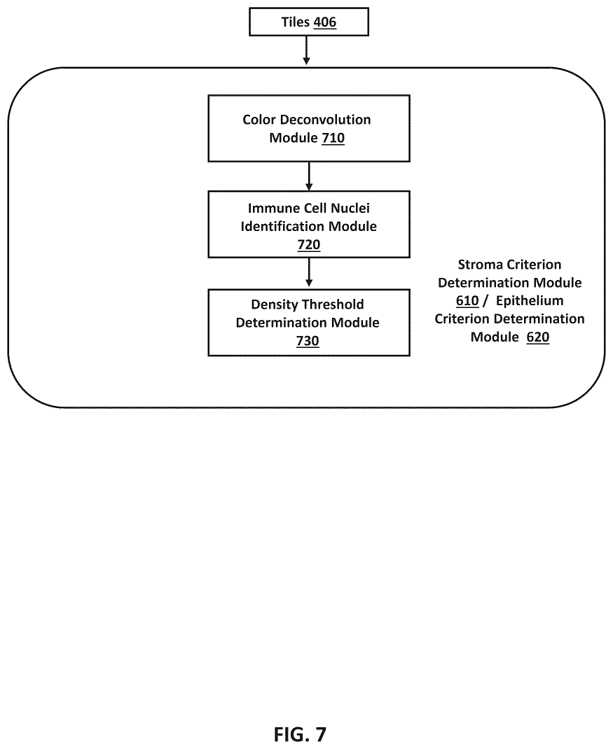

In some embodiments, stroma criterion determination module 610 and epithelium criterion determination module 620 may include one or more modules used to determine first stroma criterion result 612, second stroma criterion result 614, first epithelium criterion result 622, and second epithelium criterion result 624. As an example, with reference to FIG. 7, stroma criterion determination module 610/epithelium criterion determination module 620 is depicted. For simplicity, a single module 610/620 is displayed, however it should be understood that each of stroma criterion determination module 610 and epithelium criterion determination module 620 may include the same or similar components as described in FIG. 7. For example, both stroma criterion determination module 610 and epithelium criterion determination module 620 may include a color deconvolution module 710, an immune cell nuclei identification module 720, a density threshold determination module 730, or other components.

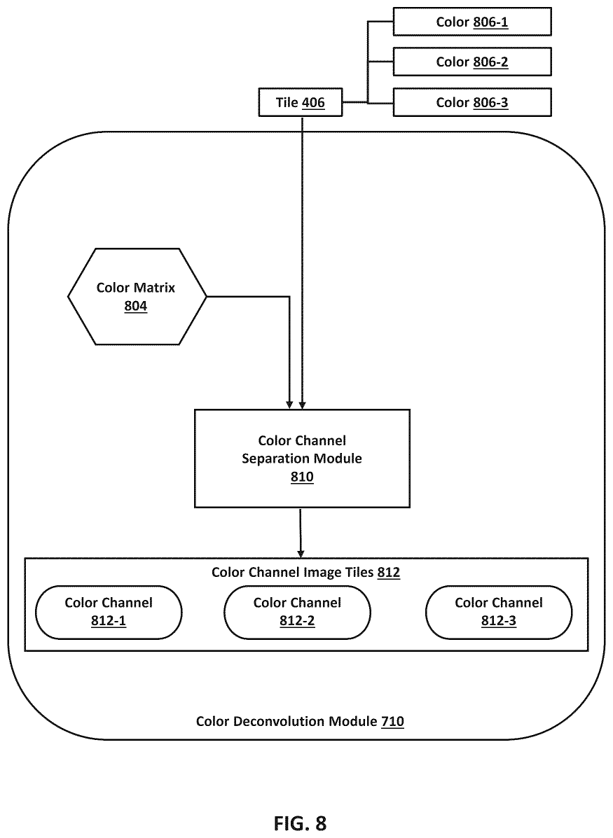

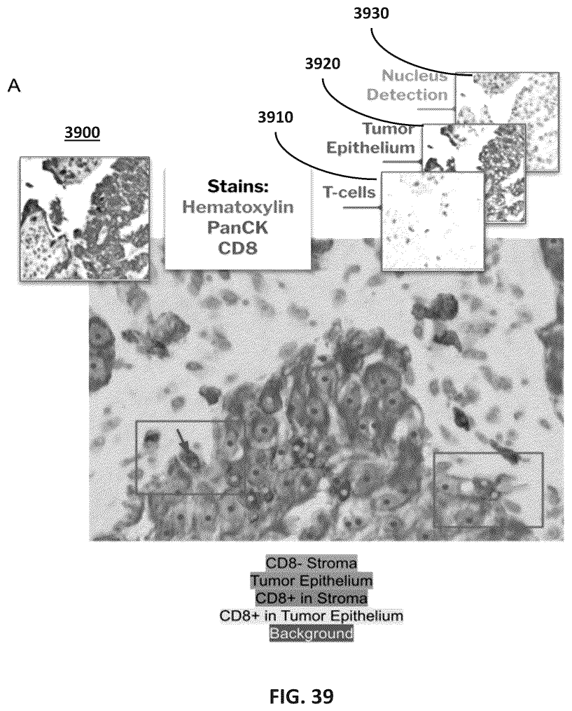

Color deconvolution module 710 may be configured to de-convolve tiles 406 or a whole slide image (e.g., digital pathology image 404) to obtain separate image tiles for each color channel. In some embodiments, color deconvolution module 710 may perform a color deconvolution to generate a plurality of color channels from an input image tile, where a first color channel highlights immune cells and a second color channel distinguishes tumor epithelium from tumor stroma. As an example, with reference to FIG. 8, color deconvolution module 710 may take tile 406, formed of L colors, and may output I color channels. For example, tile 406 may be composed of three main colors or color groups 806-1, 806-2, and 806-3 (collectively “colors 806”). Each of color channels 812 may correspond to one of colors 806 forming tile 406. Color channels 812 may be of a same size and shape as tile 406. The number of color channels may depend on a number or type of staining agent used to stain the biological sample from which image tile 406 is derived. For example, if three staining agents are used (e.g., hematoxylin, panCK, CD) then color channel separation module 810 may generate three color channels each highlighting a different aspect of tile 406. As another example, different staining agents, such as H&E, panCK, CD8, DAB, FastRed, FastBlue, Giemsa, Feulgen, Light Green, H&E DAB, Masson Trichrome, or other staining agents, or combinations thereof, may be applied to a biological sample (e.g., automated staining system 230 of FIG. 2 may be used to apply one or more staining agents to a biological sample). When the stained slides are imaged by image scanner 240, the WSI produced may include color channels that depict the different staining agents (e.g., RGB). The staining agents may be selected to highlight different features. For example, pan-cytokeratin (panCK) may be used to highlight tumor epithelium. Cluster of differentiation 8 (CD8) may be used to highlight immune cells. Hematoxylin may be used to highlight cell nuclei, extracellular matrix, and/or cell cytoplasm. As an example, with reference to FIG. 39, image tile 3900 may depict a biological sample stained with hematoxylin, panCK, and CD8. Color deconvolution module 710 may de-convolve the color channels of image tile 3900 to obtain an image tile 3910 highlighting immune cells within the biological sample depicted by image tile 3900, an image tile 3920 highlighting tumor epithelium within the biological sample depicted by image tile 3900, and image tile 3930 highlighting cell nuclei within the biological sample depicted by image tile 3900.

Returning to FIG. 8, tile 406 may be provided as input to color channel separation module 810, along with color matrix 804. Color matrix 804, in some embodiments, may be pre-generated based on the types of staining agents being used. For example, for RGB color images, color matrix 804 may be a three-color matrix M representing an absorbance level of red, green, and blue light in saturated regions of each dye on its own. In some embodiments, color channel separation module 810 may be configured to normalize and invert color matrix 804 to obtain a translated version of the matrix, translation matrix M^{T}. Color channel separation module 810 may use translation matrix M^{T} to compute a contribution of each stain based on the red, green, and blue values of the pixels in image tile 406. The contribution from each stain may be output as a separate color channel 812.

Returning to FIG. 7, immune cell nuclei identification module 720 may be configured to identify a nucleus of each immune cell within an image tile. In some embodiments, the color deconvolution process performed by color deconvolution module 710 may produce three color channels 812. For each color channel 812, immune cell nuclei identification module 720 may identify each immune cell and identify a nucleus of each immune cell. In some embodiments, immune cell nuclei identification module 720 may implement a computer vision model to detect objects depicting immune cell nuclei within tile 406 (or more specifically, one or more of color channels 812). The computer vision model, which may be a convolutional neural network, vision transformer, or other neural network for performing object recognition, may be trained to detect immune cells within an image. In some embodiments, one particular staining agent may be used to highlight immune cells. For example, CD8 may be used to highlight immune cells. Immune cell nuclei identification module 720 may analyze one of color channels 812 highlighting immune cells (e.g., the color channel depicting brown, as CD8 is designed to stain immune cells brown). Based on the analysis of that color channel 812, immune cell nuclei identification module 720 may determine a pixel location within a corresponding tile 406 of each detected immune cell. As mentioned above, a computer vision model may be used to detect the immune cells and output their pixel coordinates. In some embodiments, a trained pathologist may detect the immune cells and may record their pixel coordinates. In some embodiments, the trained pathologist may use the computer vision model's output and may refine the results based on their analysis.

In some embodiments, distance measurements may be computed based on the detected immune cells and their nuclei. The distances may indicate how far a given immune cell (or immune cell nucleus) is from the ESI. The distance indicates how far a given immune cell needs to travel to enter the tumor epithelium, where it can kill tumor cells. As an example, with reference to FIG. 28A, density threshold determination module 730 may determine a distance xₛ from ESI 2806 to immune cell 2808 and/or intra-cellular structures 2810.

In some embodiments, density threshold determination module 730 may be configured to determine a stroma-immune cell density threshold and an epithelium-immune cell density threshold. The stroma-immune cell density threshold and the epithelium-immune cell density threshold may be determined based on a number of immune cells determined to be present at the ESI. The stroma-immune cell density threshold and the epithelium-immune cell density threshold may be determined based on the number of immune cells determined to be present at the ESI divided by the total number of tiles (e.g., Nt). The stroma-immune cell density threshold and the epithelium-immune cell density threshold may be determined based on distributions of the immune cells. For example, the stroma-immune cell density threshold may be determined based on distribution 2832 describing the behavior of the immune cells in the tumor stroma, as illustrated by histogram 2830 of FIG. 28C, the epithelium-immune cell density threshold may be determined based on first distribution 2822 describing the behavior of the immune cells in the tumor epithelium, as illustrated by histogram 2820 of FIG. 28B, and the threshold ranges of epithelium-stroma interface densities may be determined based on portions 2824 and 2834 of the distributions describing the behavior of the immune cells at the ESI, as illustrated by histograms 2820 and 2830 of FIGS. 28B-28C.

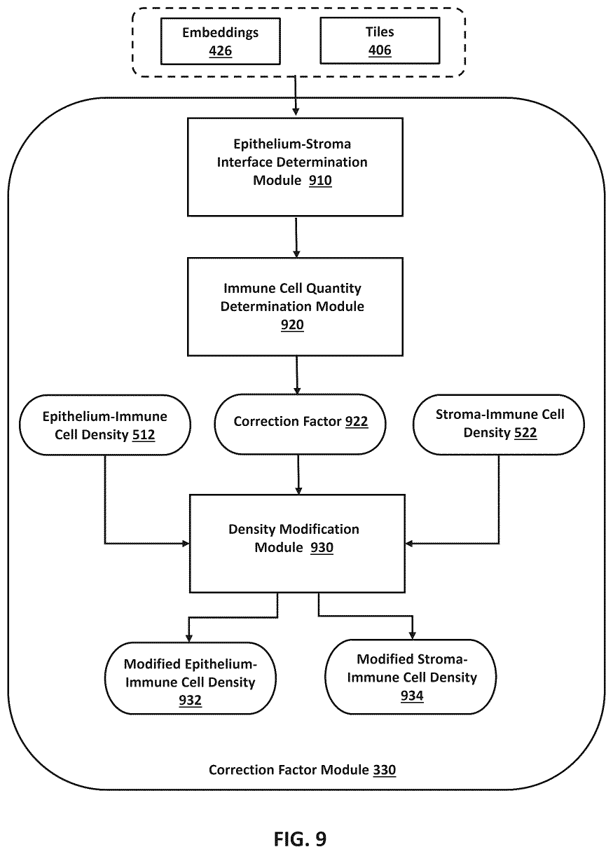

Returning to FIG. 3, correction factor module 330 may be configured to determine a correction factor and apply the determined correction factor to an image and/or image tile to remove artifacts. As an example, with reference to FIG. 9, correction factor module 330 may include an epithelium-stroma interface determination module 910, an immune cell quantity determination module 920, a density modification module 930, or other components. In some embodiments, tumor epithelial tissue can shrink away from stromal areas as a result of the histology process. Epithelium-stroma interface determination module 910 may be configured to determine a location of the ESI within tiles 406. The ESI may indicate the separation between the tumor epithelium and the tumor stroma surrounding the tumor epithelium. Immune cell quantity determination module 920 may be configured to determine a number of immune cells at the ESI and may compute a correction factor 922. In some embodiments, a computer vision model may be used by epithelium-stroma interface determination module 910 to identify the ESI within a given image tile. In some embodiments, a same or an additional computer vision model may be used by immune cell quantity determination module 920 to identify immune cells at the ESI

Density modification module 930 may be configured to determine how, if at all, to modify the calculated stroma-immune cell density, the epithelium-immune cell density, or both, of tiles 406. In particular, the determined tiles 406 may correspond to tiles identified as depicting tumor epithelium and surrounding tumor stroma. In some embodiments, density modification module 930 may receive, as input, epithelium-immune cell density 512, stroma-immune cell density 522, and correction factor 922. Density modification module 930 may output a modified epithelium-immune cell density 932 and a modified stroma-immune cell density 934. Modified epithelium-immune cell density 932 and modified stroma-immune cell density 934 may represent epithelium-immune cell density 512 and stroma-immune cell density 522, respectively, after correction factor 922 has been applied.

Returning to FIG. 3, immunophenotype classification module 340 may be configured to classify the tumor depicted by digital pathology image 404 into one of a predefined set of tumor immunophenotypes. As referred to herein, the terms “tumor immunophenotype” and “immunophenotype” are used interchangeably. Immunophenotype classification module 340 may receive inflammation type 532 from tile analysis module 320 and determine an immunophenotype of the tumor based on inflammation type 532. In some embodiments, immunophenotype classification module 340 may be configured to identify an immunotherapy that can be selected for the patient associated with the analyzed biological sample (e.g., biological sample 402). The identified immunotherapy may be recommended as a therapy to be used to treat the patient.

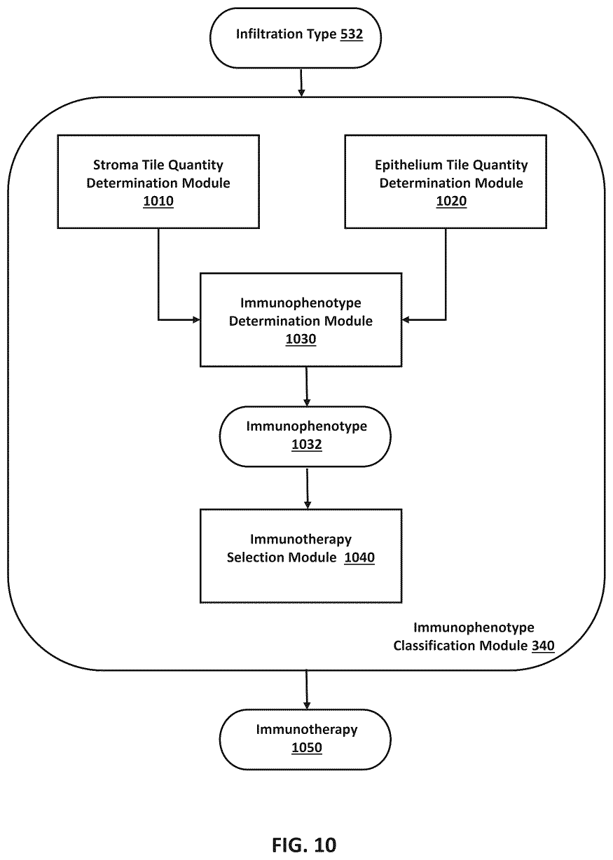

As illustrated, for example, by FIG. 10, immunophenotype classification module 340 may include a stroma tile quantity determination module 1010, an epithelium tile quantity determination module 1020, an immunophenotype determination module 1030, an immunotherapy selection module 1040, or other components. Stroma tile quantity determination module 1010 may be configured to determine a number of image tiles in the WSI of the first inflammation type. Epithelium tile quantity determination module 1020 may be configured to determine a number of image tiles in the WSI of the second inflammation type.

The number of image tiles of the first inflammation type and the number of image tiles of the second inflammation type may be input to immunophenotype determination module 1030. Immunophenotype determination module 1030 may be configured to determine an immunophenotype 1032 of an image depicting a tumor (e.g., digital pathology image 404) based on the number of tiles of the first inflammation type and the number of image tiles of the second inflammation type. The immunophenotypes that an image may be classified into include a desert immunophenotype classification, an excluded immunophenotype classification, and an inflamed or infiltrated immunophenotype classification. If the number of image tiles of the first inflammation type is less than a first threshold and the number of image tiles of the second inflammation type is less than a second threshold, then immunophenotype 1032 the desert immunophenotype classification. If the number of image tiles of the second inflammation type is greater than or equal to the first threshold and the number of image tiles of the second inflammation type is less than the second threshold, immunophenotype 1032 may be the excluded immunophenotype classification. If the number of image tiles of the first inflammation type is greater than or equal to the first threshold and the number of image tiles of the second inflammation type is greater than or equal to the second threshold, immunophenotype 1032 may be inflamed or infiltrated immunophenotype classification.

Immunotherapy selection module 1040 may be configured to select an immunotherapy 1050 to be recommended for a patient based on immunophenotype 1032. In some embodiments, immunotherapy 1050 may include providing a patient with a particular therapeutic. For example, atezolizumab may be a therapeutic provided to patients based on immunophenotype 1032. Depending on immunophenotype 1032—desert, excluded, inflamed—a particular immunotherapy may be selected as a recommend treatment for the corresponding patient.

Output generation module 350 may be configured to generate outputs corresponding to tiles 406 and/or digital pathology image 404. In some embodiments, the outputs may be generated based on a user request. As described herein, the output can include a variety of visualizations, interactive graphics, and reports based upon the type of request and the type of data that is available. In some embodiments, the output will be provided to user device 130 for display. In some embodiments, the output can be accessed directly from digital pathology image generation subsystem 110. The output may be based on existence of and access to the appropriate data. Thus, output generation module 350) may access metadata and anonymized patient information as needed. As with the other modules of epithelium/stroma image processing subsystem 112, output generation module 350 can be updated and improved in a modular fashion, so that new output features can be provided to users without requiring significant downtime.

The general techniques described herein can be integrated into a variety of tools and use cases. For example, as described, a user (e.g., pathologist or clinician) can access user device 130, which may be in communication with digital pathology image generation subsystem 110, epithelium/stroma image processing subsystem 112, model training subsystem 114, epithelium-stroma interface image processing subsystem 116, or other components. In some embodiments, user device 130 may provide a digital pathology image for analysis. Digital pathology image generation subsystem 110 epithelium/stroma image processing subsystem 112, model training subsystem 114, and epithelium-stroma interface image processing subsystem 116, and/or the functionalities implemented by digital pathology image generation subsystem 110, epithelium/stroma image processing subsystem 112, model training subsystem 114, and epithelium-stroma interface image processing subsystem 116, may be provided as a standalone software tool or package that searches for corresponding matches, identifies similar features, and generates appropriate output for the user upon request. As a standalone tool or plug-in that can be purchased or licensed on a streamlined basis, the tool can be used to augment the capabilities of a research or clinical lab. Additionally, the tool can be integrated into the services made available to a user of computing system 102. For example, the tool can be provided as a unified workflow, where a user who conducts or requests a whole slide image to be created automatically receives a report of noteworthy features within the image and/or similar whole slide images that have been previously indexed. Therefore, in addition to improving whole slide image analysis, the techniques can be integrated into existing systems to provide additional features not previously considered or possible.

Moreover, one or more machine learning models implemented by digital pathology image generation subsystem 110, epithelium/stroma image processing subsystem 112, model training subsystem 114, and/or epithelium-stroma interface image processing subsystem 116, can be trained and customized for use in particular settings. For example, a machine learning model implemented by tile embedding module 420 can be specifically trained for use in providing insights relating to specific types of tissue (e.g., lung, heart, blood, liver, etc.). As another example, the machine learning model implemented by tile embedding module 420 can be trained to assist with safety assessment, for example in determining levels or degrees of toxicity associated with drugs or other potential therapeutic treatments. Once trained for use in a specific subject matter or use case, the machine learning model implemented by tile embedding module 420 is not necessarily limited to that use case. Training may be performed in a particular context, e.g., toxicity assessment, due to a relatively larger set of at least partially labeled or annotated images.

Returning to FIG. 1, model training subsystem 114 may be configured to train one or more machine learning models used by digital pathology image generation subsystem 110, epithelium/stroma image processing subsystem 112, and/or epithelium-stroma interface image processing subsystem 116. For example, tile embedding module 420 of FIG. 4 may implement one or more machine learning models trained using model training subsystem 114. Model training subsystem 114 may be configured to perform a training process to training machine learning models, which may then be deployed by other components (e.g., tile embedding module 420, epithelium-immune cell density calculation module 510, stroma-immune cell density calculation module 520, etc.).

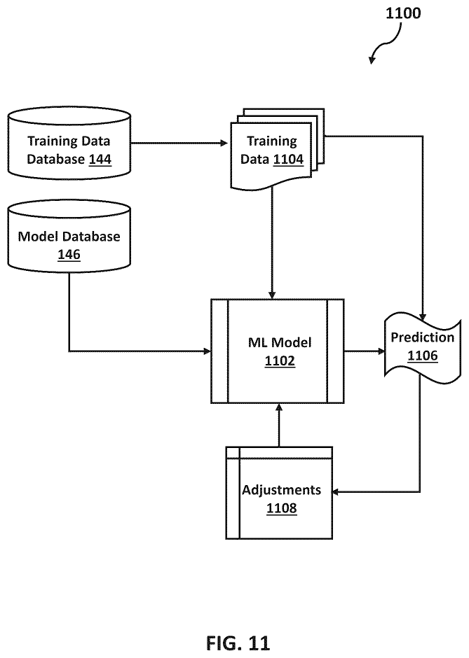

As an example, with reference to FIG. 11, model training subsystem 114 may be configured to perform process 1100 to train a machine learning model 1102. Persons of ordinary skill in the art will recognize that other training processes may be used to train a machine learning model used by components of system 100. For example, some models may use contrastive learning. Thus, process 1100 should not be construed as limiting the disclosed embodiments to particular training processes. In process 1100, training data 1104 may be retrieved from training data database 144. Different training data may be used to train different types of machine learning models. Furthermore, validation data may also be stored in training data database 144. The training data and the validation data may be identified and retrieved prior to the training process beginning.

In some embodiments, training data 1104 may include images depicting biological samples. For example, the images may depict tumor regions of patients diagnosed with NSCLC. Training data 1104 may include whole slide images. The whole slide images may be split into image tiles (using a process the same or similar to the image tiling techniques described in FIG. 4). Training data 1104 may include these image tiles. Model training subsystem 114 may select a to-be-trained machine learning model (e.g., machine learning model 1102), which may be retrieved from model database 146. Machine learning model 1102 may be selected based on a type of biological sample being analyzed, an immunophenotype to be identified, an immunotherapy to be determined, and/or other criteria. Model training subsystem 114 may select training data 1104, which may be retrieved from training data database 144. Model training subsystem 114 may select training data 1104 from training data stored in training data database 144 based on a type of machine learning model that was selected.

Model training subsystem 114 may provide training data 1104 to machine learning model 1102. Training data 1104 may include images depicting biological samples. For example, the images may depict tumor regions of patients diagnosed with NSCLC. Training data 1104 may be input to machine learning model 1102, which may generate a prediction 1106. Prediction 1106 may indicate, amongst other information, characteristics of the biological samples depicted by the images in training data 1104.

Prediction 1106 may be compared to a ground truth identified from training data 1104. As mentioned above, the images included in training data 1104 may include labels. These labels may indicate characteristics of the biological sample (e.g., cellular structures identified). Therefore, prediction 1106 may indicate whether machine learning model 1102 correctly identified the characteristics. Model training subsystem 114 may be configured to compare a given image's label with prediction 1106 for that image. Model training subsystem 114 may further determine one or more adjustments 1108 to be made to one or more hyper-parameters of machine learning model 1102. The adjustments to the hyper-parameters may be to improve predictive capabilities of machine learning model 1102. For example, based on the comparison, model training subsystem 114 may adjust weights and/or biases of one or more nodes of machine learning model 1102. Process 1100 may repeat until an accuracy of machine learning model 1102 reaches a predefined accuracy level (e.g., 95% accuracy or greater, 99% accuracy or greater, etc.), at which point machine learning model 1102 may be stored in model database 146 as a trained machine learning model. The accuracy of machine learning model 1102 may be determined based on a number of correct predictions (e.g., prediction 1106).

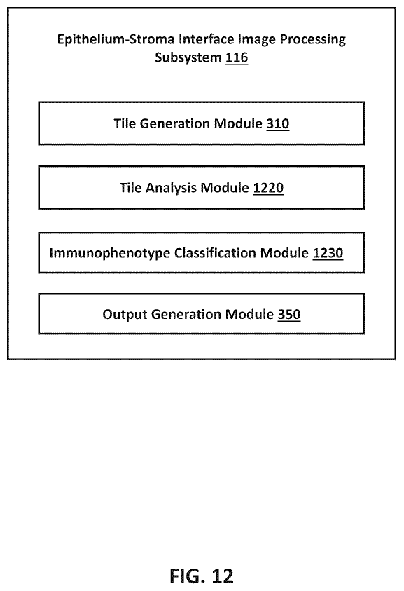

Returning to FIG. 1, computing system 102 may include epithelium-stroma interface image processing subsystem 116. Epithelium-stroma interface image processing subsystem 116 may be configured to determine a tumor immunophenotype based on a density of immune cells detected at an epithelium-stroma interface (ESI). The ESI refers to the portion of a tumor where tumor epithelium separates from tumor stroma. In some embodiments, the ESI may be identified by a trained pathologist. The trained pathologist may annotate the image to delineate portions of the image depicting tumor stroma and portions of the image depicting tumor epithelium. In some embodiments, machine learning models (e.g., a computer vision model) may be used to detect the ESI within an image, as well as annotate the image to denote the ESI. In some embodiments, the trained pathologist may use the machine learning models to detect the ESI and may subsequently adjust the detected ESI. As an example, with reference to FIG. 12, epithelium-stroma interface image processing subsystem 116 may include tile generation module 310, a tile analysis module 1210, an immunophenotype classification module 1220, output generation module 350, or other components.

Tile generation module 310, as previously described above with reference to FIGS. 3-4, may be configured to receive an image of a tumor and generate a plurality of tiles representing the image. Tile generation module 310 may divide an image, such as digital pathology image 404, into overlapping or non-overlapping tiles 406 of a predefined size. Tiles 406 may be of a smaller size than digital pathology image 404, which can enable epithelium-stroma interface image processing subsystem 116 to process tiles 406 faster than digital pathology image 404. For example, digital pathology image 404 may be of the order of 10⁵ pixels×10⁵ pixels, while tiles 406 may be of the order of 10² pixels×10² pixels. Furthermore, tiles 406 can be analyzed using parallelization techniques to further reduce processing time and computing resources.

In some embodiments, tile generation module 310 may include a tile embedding module 420 configured to generate embeddings 426 representing tiles 406. Embeddings 426 refer to representations of tiles 406 in a multi-dimensional feature space. In some embodiments, embeddings 426 may be represented by an N-dimensional vector. In some embodiments, the same or similar tile generation module 310 may be included within epithelium/stroma image processing subsystem 112 and epithelium-stroma interface image processing subsystem 116.

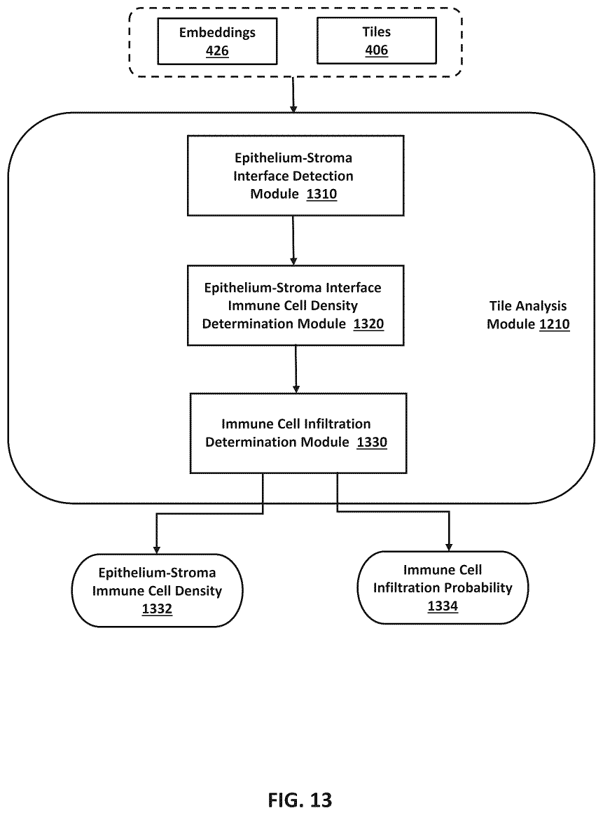

Tile analysis module 1210 may be configured to analyze tiles 406 generated by tile generation module 310. As an example, with reference to FIG. 13, tile analysis module 1210 may include an epithelium-stroma interface detection module 1310, an epithelium-stroma interface immune cell density determination module 1320, an immune cell infiltration determination module 1330, or other components. Epithelium-stroma interface detection module 1310 may be configured to detect the ESI within an image (e.g., digital pathology image 404) of a tumor and/or within tiles derived from the image. In some embodiments, epithelium-stroma interface detection module 1310 may be configured to generate an indication of the detected ESI.

Epithelium-stroma interface immune cell density determination module 1320 may be configured to determine an epithelium-stroma interface immune cell density. The ESI immune cell density may indicate a number of immune cells detected within a threshold distance of the ESI. In some embodiments, the threshold distance may include a first distance from the ESI into the tumor stroma and a second distance from the ESI into the tumor epithelium. The number of immune cells at the ESI may be computed by determining how many immune cells are located within the first distance of the ESI (e.g., into the tumor stroma) and how many immune cells are located within the second distance of the ESI (e.g., into the tumor epithelium).

Immune cell infiltration determination module 1330 may be configured to determine an immune cell infiltration probability. The immune cell infiltration probability may indicate a likelihood that an immune cell located at the ESI will penetrate the ESI and infiltrate the tumor epithelium. The higher the immune cell infiltration probability, the more immune cells that will enter the tumor epithelium and attack cancer cells.

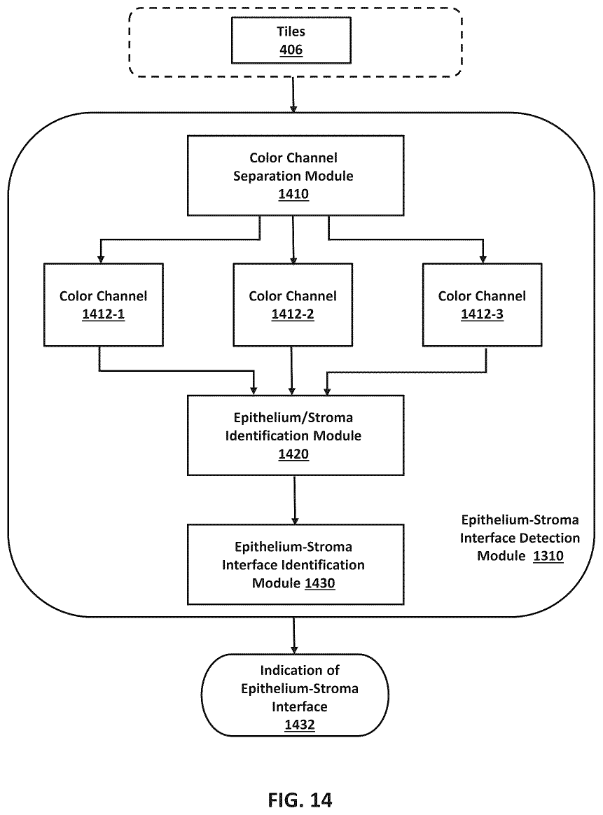

Epithelium-stroma interface detection module 1310 may be configured to detect the presence of the ESI within image tiles. In some embodiments, epithelium-stroma interface detection module 1310 may resolve a boundary of each tumor within the whole slide image (e.g., digital pathology image 404) based on the detected locations of the ESI within the image tiles. As an example, with reference to FIG. 14, epithelium-stroma interface detection module 1310 may include a color channel separation module 1410, an epithelium/stroma identification module 1420, an epithelium-stroma interface identification module 1430, or other components. Epithelium-stroma interface detection module 1310 may be configured to receive tiles 406 from tile generation module 310. Tiles 406 may be provided individually, however alternatively multiple tiles 406 may be provided to epithelium-stroma interface detection module 1310. In some embodiments, embeddings 426 may also be provided to epithelium-stroma interface detection module 1310.

As mentioned previously, digital pathology image 404 may a tumor that has had one or more stains applied. Each stain may cause different aspects of the tumor to be highlighted. For example, a panCK stain may be used to highlight tumor epithelium, a CD8 stain may be used to highlight immune cells, and a hematoxylin stain may be used to highlight cell nuclei, extracellular matrices, and/or cell cytoplasm. Each stain may highlight aspects of the tumor in a different color. For example, the CD8 stain may highlight immune cells in brown, while the panCK stain may highlight tumor epithelium in purple. Color channel separation module 1410 may be configured to separate each of tiles 406 into one or more color channels 1412-1, 1412-2, 1412-3 (collectively “color channels 1412”). Color channels 1412 may correspond to the number of stains applied to the tumor depicted by tiles 406. For example, if panCK. CD8, and hematoxylin stains are used, color channel separation module 1410 may produce three color channels 1412, each highlighting specific aspects of the tumor. In some embodiments, color channel separation module 1410 may be the same or similar to color deconvolution module 710 of FIG. 8, and the previous description may apply.

Epithelium/stroma identification module 1420 may be configured to identify portions of tumor epithelium and portions of tumor stroma within tiles 406. In some embodiments, tumor stroma and tumor epithelium may be detected using color channels 1412. For example, one color channel may be used to highlight tumor epithelium while another color channel may be used to highlight tumor stroma. In some embodiments, epithelium/stroma identification module 1420 may analyze the color channel highlighting tumor epithelium and may resolve the pixel locations associated with tumor epithelium. Similarly, epithelium/stroma identification module 1420 may analyze the color channel highlighting tumor stroma and may resolve the pixel locations associated with tumor stroma. As an example, with reference to FIGS. 20A-20C, whole slide image 2000 may depict a tumor that has been stained using three stains, hematoxylin, panCK, and CD8. Each stain causes particular cell structures to be highlighted in a different color—hematoxylin highlighting tumor stroma in blue, panCK highlighting tumor epithelium in purple/pink, and CD8 highlighting immune cells in brown. Whole slide image 2020 of FIG. 20B illustrates locations of pixels associated with tumor stroma. Each data point in whole slide image 2020 has an x-y pixel coordinate that relates to a location of that pixel within whole slide image 2000. Whole slide image 2040 of FIG. 20C illustrates locations of pixels associated with tumor epithelium in red. Epithelium/stroma identification module 1420 may associate each pixel in whole slide image 2000 with tumor stroma (illustrated in whole slide image 2020) or tumor epithelium (illustrated in whole slide image 2040). A bitmap indicating, for each pixel of whole slide image 2000, whether tumor epithelium or tumor stroma is depicted may be generated by epithelium/stroma identification module 1420. In some embodiments, a data structure including a listing of pixels (e.g., pixel 1-pixel N), each pixel's location within the whole slide image, and a flag indicating whether that pixel depicts tumor stroma, tumor epithelium, or neither, and/or other information may be output by epithelium/stroma identification module 1420.

In some embodiments, epithelium-stroma interface identification module 1430) may be configured to determine the ESI based on the locations of the pixels associated with tumor stroma and the locations of the pixels associated with the tumor epithelium. For example, epithelium-stroma interface identification module 1430 may receive the data structure from epithelium/stroma identification module 1420 and may determine one or more boundaries formed around clusters of pixels associated with tumor epithelium and/or tumor stroma. Based on the boundaries, epithelium-stroma interface identification module 1430 may determine where the ESI is located with respect to each tile and/or the whole slide image. Furthermore, epithelium-stroma interface identification module 1430) may generate an indication 1432 of the ESI, which can be used to render a graphical depiction of the boundary overlayed on the whole slide image and/or one or more tiles derived from the whole slide image.

In some embodiments, epithelium-stroma interface identification module 1430) may implement one or more machine learning model to identify the ESI. For instance, the machine learning models may analyze a gradient of the pixels to detect a transition tumor stroma to tumor epithelium, or vice versa. In some embodiments, the machine learning models may use edge detection techniques in computer vision to identify a boundary of a cluster of pixels associated with tumor epithelium. For example, a Sobel filter, Laplacian filter, and/or Canny filter may be used to perform edge detection on a tile level and/or whole slide image level. This boundary may be used to determine the ESI. In some embodiments, epithelium-stroma interface identification module 1430 may output indication 1432 of the ESI. Indication 1432 of the ESI may be used, in some embodiments, to annotate tiles 406 and/or digital pathology image 404 to depict ESI.

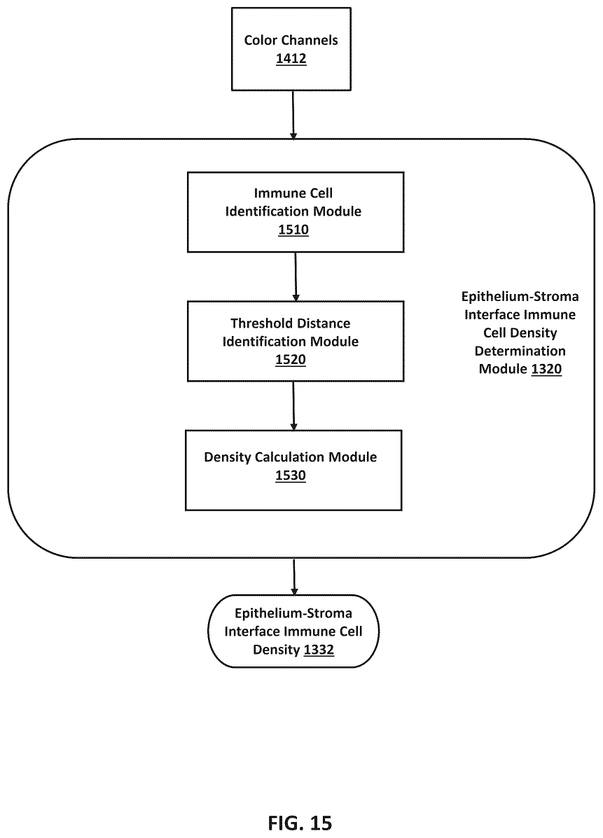

Epithelium-stroma interface immune cell density determination module 1320 may be configured to determine an epithelium-stroma interface immune cell density. The epithelium-stroma interface immune cell density indicates a number of immune cells that are located within a threshold distance of the ESI. The epithelium-stroma interface immune cell density may be determined on a tile-by-tile basis. In some embodiments, the epithelium-stroma interface immune cell density may be determined based on the number of immune cells identified as being within a threshold distance of the ESI as depicted within a given tile. As an example, with reference to FIG. 15, epithelium-stroma interface immune cell density determination module 1320 may include an immune cell identification module 1510, a threshold distance identification module 1520, a density calculation module 1530, or other components.

Immune cell identification module 1510 may be configured to detect immune cells within a color channel. In particular, if a stain is used to specifically highlight immune cells, a color channel associated with that stain may be analyzed. For example, the CD8 stain turns immune cells brown, so the color channel to analyze would be the brown color channel. In some embodiments, immune cell identification module 1510 may implement one or more machine learning models trained to recognize objects, such as immune cells, within an image tile and may determine a size (e.g., diameter, area, circumference, etc.) of each recognized immune cell. For example, the machine learning models may include a convolutional neural network, vision transformer, or other computer vision models. These models may be trained using images of immune cells. Alternatively, the models may be pre-trained on non-medical images (e.g., using images from the ImageNet database) and then fine-tuned on images depicting immune cells. The training images may also be image tiles. In some embodiments, the training of the models may be supervised, semi-supervised, or unsupervised. In some embodiments, the immune cells may be detected by a trained pathologist. In some embodiments, the trained pathologist may use the machine learning models to perform an initial identification of immune cells within a tile, which can then be reviewed and edited by the trained pathologist. Immune cell identification module 1510 may generate a data structure indicating a location of each detected immune cell. Alternatively, or additionally, immune cell identification module 1510 may update the data structure generated by epithelium/stroma identification module 1420 to indicate which pixels depict immune cells. The location may be a 2-dimensional point in pixel space (e.g., with respect to the analyzed image tile). In some embodiments, the data structure may include an approximate size of the detected immune cells, a distance of that immune cell from the ESI, an indication of whether the immune cell is located within tumor epithelium or tumor stroma, or other information.

Threshold distance identification module 1520 may be configured to determine a threshold distance from the ESI to be used for computing the epithelium-stroma interface immune cell density. Immune cells detected within the threshold distance of the ESI may be used to determine an immune cell density at the ESI. Immune cells further from the ESI, either into the tumor stroma or the tumor epithelium, may be used for calculating a stroma-immune cell density or an epithelium-immune cell density, respectively. In some embodiments, threshold distance identification module 1520 may determine the threshold distance based on a size of an immune cell. For example, each immune cell may have an approximate diameter of 10 microns. In some embodiments, threshold distance identification module 1520 may calculate the threshold distance based on an analysis of the other immune cells detected within the image tiles of the whole slide image. Threshold distance identification module 1520 may determine the threshold distance based on the size of the detected immune cells. For example, threshold distance identification module 1520 may determine an average size of the immune cells detected by the machine learning models of immune cell identification module 1510) and may set the threshold distance as a multiple of the average immune cell size (e.g., x_{threshold}=x_{avg}, x_{threshold}=2 x_{threshold}=x_{avg}, x_{threshold}=5 x_{avg}, etc.). In some embodiments, threshold distance identification module 1520 may determine the threshold distance based on an average size of immune cells detected from the training data used to train the machine learning models of immune cell identification module 1510. In some embodiments, threshold distance identification module 1520 may select the threshold distance from a set of predefined threshold distances, however the threshold distance may be configurable.

Density calculation module 1530 may be configured to calculate epithelium-stroma interface immune cell density 1332 based on the number of immune cells determined to be within the threshold distance x_{threshold}. For example, with reference to FIG. 28A, immune cells 2808 and 2812 may be within threshold distance x_{threshold} of ESI 2806. Therefore, immune cells 2808 and 2812 may be included when computing epithelium-stroma interface immune cell density 1332. If threshold distance x_{threshold} is less than distance threshold distance xₛ, immune cells 2808 may not be included in the calculation of epithelium-stroma interface immune cell density 1332. In some embodiments, threshold distance x_{threshold} may include a first threshold distance x_{s-threshold} into the tumor stroma and a second threshold distance Xe-threshold into the tumor epithelium. Threshold distances x_{s-threshold} and x_{e-threshold} may be the same or similar to the distance into the tumor epithelium. As mentioned above, threshold distance x_{threshold} may be determined based on the size of the immune cells. In some examples, the threshold distance may be selected to be 2-3 standard deviations of the mean immune cell size.

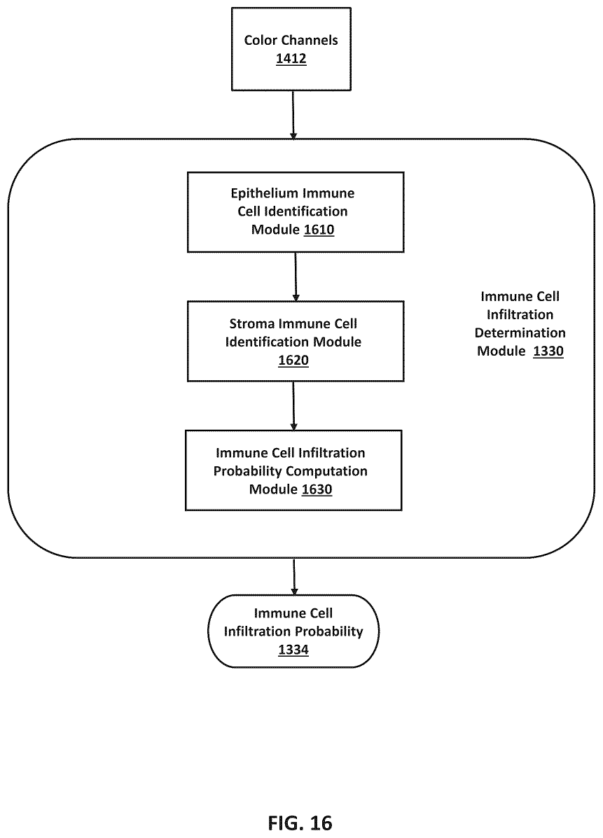

Immune cell infiltration determination module 1330 may be configured to determine an immune cell infiltration probability. The immune cell infiltration probability may be determined based on a likelihood of immune cells in the tumor stroma infiltrating the tumor epithelium. The immune cell infiltration probability may be based on a number of immune cells in the tumor stroma and a number of immune cells in the tumor epithelium. For example, a number of immune cells detected within the tumor epithelium of a given image tile may be determined and a number of immune cells detected within the tumor stroma of the same image tile may be determined. The ratio of the number of immune cells detected within the tumor epithelium to the number of immune cells detected within the tumor stroma may represent the likelihood of an immune cell penetrating the ESI and infiltrating the tumor epithelium. As an example, with reference to FIG. 16, immune cell infiltration determination module 1330 may include an epithelium immune cell identification module 1610, a stroma immune cell identification module 1620, an immune cell infiltration probability computation module 1630, or other components.

Epithelium immune cell identification module 1610 may be configured to identify immune cells within the tumor epithelium. In some embodiments, this may include analyzing a tile or tiles highlighting immune cells and highlighting tumor epithelium. For example, a panCK-CD8 dual-stain may be used. As seen with reference to FIG. 28A, the panCK stain may highlight tumor epithelium in one color (e.g., purple/pink) while the CD8 stain may highlight immune cells in another color (e.g., brown). Epithelium immune cell identification module 1610 may be configured to determine a pixel location of each immune cell detected within the tumor epithelium and generate a data structure storing each immune cell's pixel location. For example, with reference to FIGS. 20A and 20C, an immune cell may be identified within the tumor epithelium at location (px2, py2). Therefore, the data structure may include an identification of the immune cell as well as the pixel location. Alternatively. or additionally, the data structure generated by epithelium/stroma identification module 1420 and/or updated by immune cell identification module 1510 may be updated to indicate, for each pixel, whether that pixel depicts at least a portion of an immune cell. An identifier for each immune cell may also be stored in the data structure in association with the pixel.

In some embodiments, epithelium-immune cell identification module 1610 may aggregate the immune cells determined to be within the tumor epithelium to compute a quantity of immune cells within the tumor epithelium. In some embodiments, epithelium-immune cell identification module 1610 may generate a histogram indicating a number of immune cells in the tumor epithelium as a function of distance from the ESI. For example, first distribution 2822 of histogram 2820 of FIG. 28B may indicate a number of immune cells detected at different distances from the ESI. In some embodiments, the number of immune cells within the tumor epithelium may be determined by integrating first distribution 2822 (e.g., integrating Equation I across first distribution 2822). In some embodiments, each bin of histogram 2820 may be equal to the average size of an immune cell. In some embodiments, each bin of histogram 2820 may be selected from a predefined set of bin sizes, such as, for example, 1 micron or less, 2 microns or less, 4 microns or less, 10 microns or less, etc.

Stroma immune cell identification module 1620 may be configured to identify immune cells within the tumor stroma. In some embodiments, this may include analyzing a tile or tiles highlighting immune cells and highlighting tumor stroma. For example, a CD8 stain may be used to highlight immune cells, and a hematoxylin stain may be used to highlight tumor stroma. Stroma immune cell identification module 1620 may be configured to determine a pixel location of each immune cell detected within the tumor stroma and generate a data structure storing each immune cell's pixel location. For example, with reference to FIGS. 20A and 20B, an immune cell may be identified within the tumor stroma at location (px1, py1). Therefore, the data structure may include an identification of the immune cell as well as the pixel location. Alternatively, or additionally, the data structure generated/updated by epithelium/stroma identification module 1420, immune cell identification module 1510, and/or epithelium immune cell identification module 1610 may be updated to indicate, for each pixel, whether that pixel depicts at least a portion of an immune cell in the tumor stroma. An identifier for each immune cell may also be stored in the data structure in association with the pixel.

In some embodiments, stroma immune cell identification module 1620 may aggregate the immune cells determined to be within the tumor stroma to compute a quantity of immune cells within the tumor stroma. In some embodiments, stroma immune cell identification module 1620 may generate a histogram indicating a number of immune cells found in the tumor stroma as a function of distance from the ESI. For example, distribution 2832 of histogram 2830 of FIG. 28C may indicate a number of immune cells detected at different distances from the ESI. In some embodiments, the number of immune cells within the tumor stroma may be determined by integrating distribution 2832 (e.g., integrating Equation 2 across distribution 2832). In some embodiments, each bin of histogram 2830 may be equal to the average size of an immune cell. In some embodiments, each bin of histogram 2830 may be selected from a predefined set of bin sizes, such as, for example, 1 micron or less. 2 microns or less, 4 microns or less, 10 microns or less, etc.

Immune cell infiltration probability computation module 1630 may be configured to compute immune cell infiltration probability 1334 based on the number of immune cells identified within the tumor epithelium by epithelium immune cell identification module 1610 and the number of immune cells identified within the tumor stroma by stroma immune cell identification module 1620. In some embodiments, immune cell infiltration probability 1334 may be determined based on a ratio of the number of immune cells identified within the tumor epithelium to the number of immune cells identified within the tumor stroma. In some embodiments, immune cell infiltration probability 1334 may be determined based on the number of immune cells identified within the tumor stroma to the number of immune cells identified within the tumor epithelium. Immune cell infiltration probability 1334 may indicate the likelihood that an immune cell in the tumor stroma will penetrate the ESI and infiltrate the tumor epithelium.

Returning to FIG. 12, epithelium-stroma interface image processing subsystem 116 may include immunophenotype classification module 1230). Immunophenotype classification module 1230 may be configured to determine an immunophenotype of the tumor depicted by the whole slide image (e.g., digital pathology image 404 of FIG. 4). The tumor immunophenotype may be determined based on the epithelium-stroma interface immune cell density and the immune cell infiltration probability. As previously discussed, the tumor immunophenotype may be desert, excluded, or infiltrated/inflamed. These immunophenotypes may be referred to as the Harmut Koeppen (HK) immunophenotypes. These immunophenotypes are similar to the immunophenotypes determined by epithelium/stroma image processing subsystem 112. In some embodiments, patients whose tumors are classified as being in one HK immunophenotype may receive one type of immunotherapy, while patients whose tumors are classified into another HK immunophenotype may receive another type of immunotherapy.

Alternatively, as described below; tumors may be classified into one of a set of tumor immunophenotypes. The set of tumor immunophenotypes may include a first tumor immunophenotype and a second tumor immunophenotype. In some embodiments, an image may be classified as depicting a tumor of the first tumor immunophenotype or the second tumor immunophenotype based on a median epithelium-stroma interface immune cell density. For example, images may be classified as depicting a tumor of the first tumor immunophenotype based on the epithelium-stroma interface immune cell density being less than the median epithelium-stroma interface immune cell density. As another example, images may be classified as depicting a tumor of the second tumor immunophenotype is based on the epithelium-stroma interface immune cell density being greater than or equal to the median epithelium-stroma interface immune cell density.

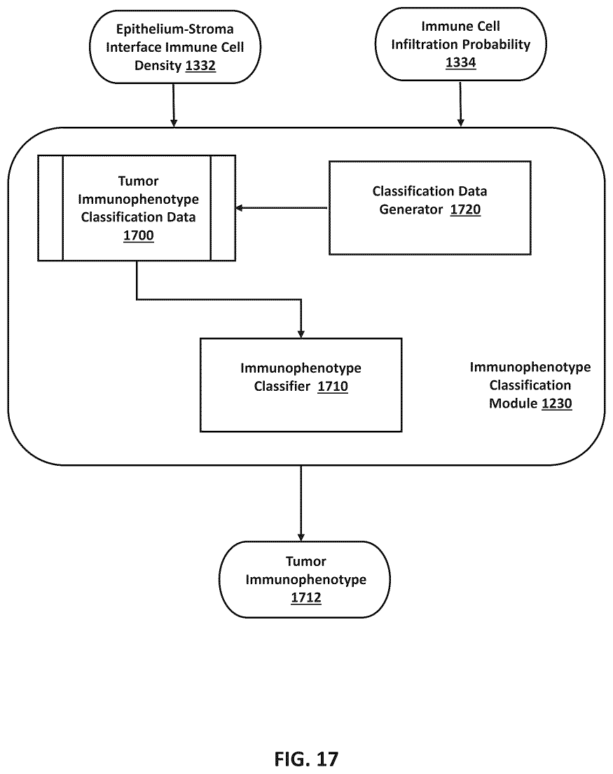

As an example, with reference to FIG. 17, immunophenotype classification module 1230 may include an immunophenotype classifier 1710, a classification data generator 1720, or other components. Immunophenotype classifier 1710 may be a trained classifier that assigns a tumor immunophenotype 1712 to an image of a tumor (e.g., digital pathology image 404). Tumor immunophenotype 1712 may be determined from a set of tumor immunophenotypes. The set of immunophenotypes may include desert, excluded, and inflamed. In some embodiments, immunophenotype classifier 1710 may determine a tumor immunophenotype classification based on epithelium-stroma interface immune cell density 1332, immune cell infiltration probability 1334, and tumor immunophenotype classification data 1700. Classification data generator 1720 may be configured to generate tumor immunophenotype classification data 1700. In some embodiments, tumor immunophenotype classification data 1700 may be stored in classification data database 148. Classification data generator 1720 may further be configured to update tumor immunophenotype classification data 1700 based on new data.

Immunophenotype classifier 1710 may determine tumor immunophenotype 1712 of an image of a tumor based on epithelium-stroma interface immune cell density 1332 and immune cell infiltration probability 1334 calculated for the image, however tumor immunophenotype 1712 may also be determined on a tile-by-tile basis. Immunophenotype classifier 1710 may compare epithelium-stroma interface immune cell density 1332 and immune cell infiltration probability 1334 to tumor immunophenotype classification data 1700. Based on the comparison, immunophenotype classifier 1710 may classify tumor immunophenotype 1712. As an example, with reference to FIG. 29, tumor immunophenotype classification data 2910 may represent different tumor immunophenotype classifications based on a computed epithelium-stroma interface immune cell density and a computed immune cell infiltration probability of an image of a tumor from one of a plurality of patients. As seen in FIG. 29, the x-axis indicates an immune cell infiltration probability (Pes) and the y-axis indicates an ESI immune cell density (cells/mm²). In some embodiments, when an image of a tumor of a patient is scanned by image scanner 240 (e.g., digital pathology image 404), epithelium-stroma interface image processing subsystem 116 may determine an immune cell density at the ESI (e.g., epithelium-stroma interface immune cell density 1332) and an immune cell infiltration probability (e.g., immune cell infiltration probability 1334) for that image. In some embodiments, the image's immune cell density at the ESI and immune cell infiltration probability may be mapped to tumor immunophenotype classification data 1700. For example, epithelium-stroma interface immune cell density 1332 and immune cell infiltration probability 1334 may be mapped to tumor immunophenotype classification data 2910 of FIG. 29. Based on the mapping, immunophenotype classifier 1710 may determine tumor immunophenotype 1712.

In some embodiments, tumor immunophenotype 1712 may selected from a set of tumor immunophenotypes. For example, the set of immunophenotypes may include the HK immunophenotypes of desert, excluded, and inflamed. As another example, the set of immunophenotypes may include a first tumor immunophenotype and a second tumor immunophenotype determined based on a median epithelium-stroma interface immune cell density.

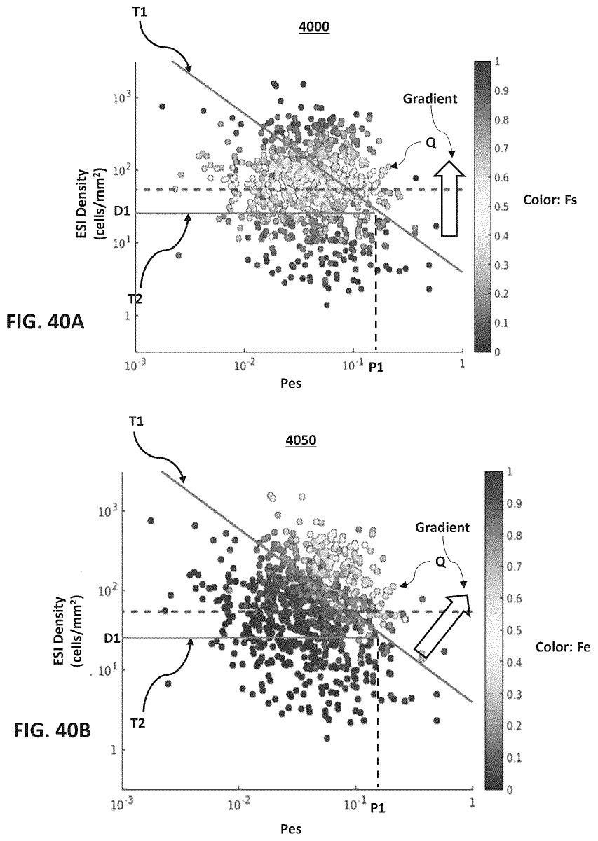

Tumor immunophenotype classification data 2910) may include three groupings of data points separated by lines T1 and T2. In some embodiments, lines T1 and T2 may be correspond to a threshold fraction of tiles (F_{sthreshold}) in the tumor stroma that are classified as being inflamed and a fraction of tiles (Fe_{threshold}) in the tumor epithelium that are classified as being of inflamed. For example, with reference to FIGS. 40A-40B, plots 4000 and 4050 respectively illustrate a fraction of tiles (Fs) classified as being inflamed in the tumor stroma and a fraction of tiles (Fe) classified as being inflamed in the tumor epithelium.

In some embodiments, to resolve the immunophenotype of an image, the fraction of tiles Fs classified as being inflamed in the tumor stroma and the fraction of tiles Fe classified as being inflamed in the tumor epithelium can be mapped to tumor immunophenotype classification data, such as tumor immunophenotype classification data 2300 of FIG. 23. As an example, consider the point Q in plots 4000 and 4050 of FIGS. 40A-40B. Point Q may correspond to an image of a tumor. To determine the tumor immunophenotype for the image, the fraction of image tiles in the tumor stroma classified as being inflamed may be determined using plot 4000 (e.g., approximately Fs=0.6) and the fraction of image tiles in the tumor epithelium that are classified as being inflamed may be determined using plot 4050 (e.g., approximately Fe=0.5). These two values (e.g., Fs=0.6, Fe=0.5) may be mapped to tumor immunophenotype classification data 2300 of FIG. 23 to determine the tumor immunophenotype of the image represented by point Q. For example, the fraction of tiles Fs in the tumor stroma classified as being inflamed and the fraction of tiles Fe in the tumor epithelium classified as being inflamed to tumor immunophenotype classification data 2300 may map to point X=(Fe, Fs). Point X may reside in region 2306 representing the tumor immunophenotype classification inflamed. Therefore, the tumor depicted by the image represented by point Q may be assigned the tumor immunophenotype of inflamed.

Tumor immunophenotype classification data 2300 may include three regions associated with the three HK tumor immunophenotype classifications: desert in region 2302, excluded in region 2304, and inflamed in region 2306. In some embodiments, the desert classification may include images where the fraction of image tiles classified as being inflamed in the tumor stroma is less than a first threshold, and the fraction of image tiles classified as being inflamed in the tumor epithelium is less than a second threshold. For example, desert in region 230) 2 includes images having Fe<Fe_{Threshold} and Fs<Fs_{Threshold}. In some embodiments, the excluded classification may include images where the fraction of image tiles classified as being inflamed in the tumor stroma is greater than or equal to the first threshold and the fraction of image tiles classified as being inflamed in the tumor epithelium is less than the second threshold. For example, excluded in region 2304 may include images having Fe<Fe_{Threshold} and Fs≥Fs_{Threshold}. In some embodiments, the inflamed classification may include images where the fraction of image tiles classified as being inflamed in the tumor stroma is greater than or equal to the first threshold and the fraction of image tiles classified as being inflamed in the tumor epithelium is greater than or equal to the second threshold. For example, inflamed in region 2306 may include images having Fe≥Fe_{Threshold} and Fs≥Fs_{Threshold}.

In some embodiments, the fraction of tiles Fs classified as being inflamed in the tumor stroma may be determined based on a number of tiles (Ns) of the total image tiles (Nt) being greater than a first threshold number (Ns_{Threshold}) of immune cells (e.g., 20% of the total image tiles). The fraction of tiles Fe classified as being inflamed in the tumor epithelium may be determined based on a number of tiles (Ne) of the total image tiles (Nt) being greater than a first threshold number (Ne_{Threshold}) of immune cells (e.g., 20% of the total image tiles).

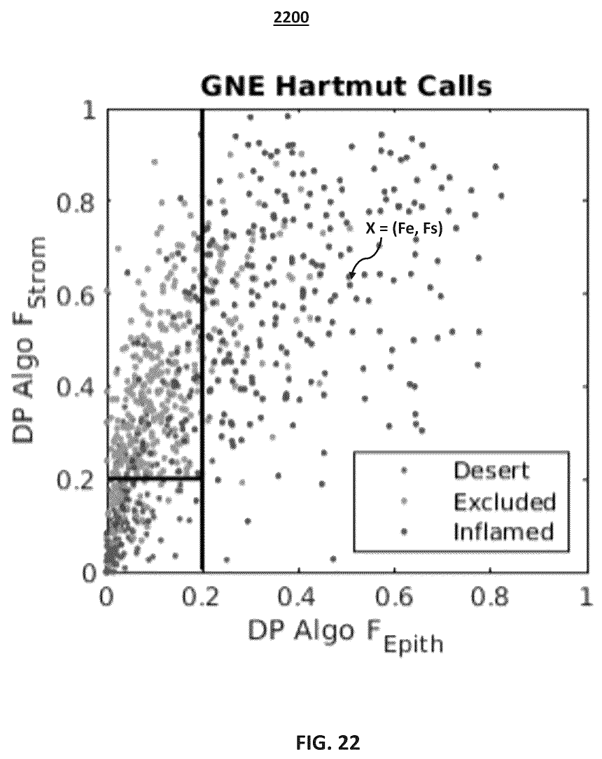

FIG. 22 illustrates plot 2200 depicting the mapped data points from plots 4000 and 4050 of FIGS. 40A-40B. As can be seen from plot 2200, data point X may represent a point determined by plotting the fraction Fe and the fraction Fs for point Q. In some embodiments, classification data generator 1720 of FIG. 17 may be configured to determine Fe_{Threshold} and Fs_{Threshold} based on the data represented within plots 4000 and 4050. For example, Fe_{Threshold} may be determined by identifying the fractions Fe and Fs including a threshold number of the data points (e.g., 80%). Classification data generator 1720 may generate tumor immunophenotype classification data 2300 of FIG. 23 based on the identified fractions Fe and Fs being set as Fe_{Threshold} and Fs_{Threshold}, respectively.

As mentioned above, in some embodiments, the tumor immunophenotype may be determined based on an epithelium-stroma interface immune cell density. In some embodiments, immunophenotype classification module 1230 may determine the tumor immunophenotype based on epithelium-stroma interface immune cell density 1332 and immune cell infiltration probability 1334. As seen in plots 4000 and 4050, the x-axis indicates a probability Pes of an immune cell located in the tumor stroma penetrating the ESI and infiltrating the tumor epithelium, and the y-axis indicates an immune cell density at the ESI. Each data point may represent an image of a tumor. In plots 4000 and 4050, the data represents images of tumors of a plurality of patients. For each image, an immune cell density at the ESI and immune cell infiltration probability may be determined. Thus, each data point represents a different tumor image of a different patient. Plots 4000 and 4050 may also indicate fractions Fs and Fe, respectively. As can be seen in plot 4000, the color gradient is a vertical line, where data points colored blue represents lower fractions Fs of inflamed stroma tile and data points color red represent higher fractions Fs of inflamed stroma tiles. Plot 4050 of FIG. 40B also depicts the same data points, however with the color gradient skewed slightly along an approximate Fe=Fs slope.

In some embodiments, the gradients of Fs and Fe in plots 4000 and 4050 follow Fs_{Threshold} and Fe_{Threshold}. For example, by analyzing the epithelium-stroma interface immune cell density and the immune cell infiltration probability, the classical HK tumor immunophenotypes can be resolved based on the defined Fs_{Threshold} and Fe_{Threshold}. As seen in plots 4000 and 4050 of FIGS. 40A-40B, the same data points are presented, along with lines T1 and T2 respectively representing Fs_{Threshold} and Fe_{Threshold}. In plot 2900 of FIG. 29, the colors indicate tumor immunophenotype, with data points of respective Fs and Fe being darker red for greater ESI immune cell densities/probabilities and darker blue for lower densities/probabilities. For example, images where the ESI immune cell density and infiltration probability fall within group G1 may be assigned to the tumor immunophenotype classification of desert, images where the ESI immune cell density and infiltration probability fall within group G2 may be assigned to the tumor immunophenotype classification of excluded, and images where the ESI immune cell density and infiltration probability fall within group G3 may be assigned to the tumor immunophenotype classification of inflamed. These values can also be mapped back to the classical HK immunophenotype classifications, as illustrated by plot 4050 of FIG. 40B based on each data points respective Fs and Fe. Therefore, the classical technique of immunophenotyping based on immune cell density in tumor stroma and tumor epithelium can also be performed based on the immune cell density at the ESI.

The median epithelium-stroma interface immune cell density (median Mp) may serve as a predictive biomarker for classifying an image into a set of tumor immunophenotypes.

In some embodiments, the median epithelium-stroma interface immune cell density may serve as a predictive biomarker for immunophenotyping. The median epithelium-stroma interface immune cell density may be based the epithelium-stroma interface immune cell density of each of the plurality of images. For example, each image (corresponding to a data point in plot 2900 of FIG. 29, plot 3000 of FIG. 30, plots 4000, 4050 of FIGS. 40A-40B) of the tumor is classified into one of the set of tumor immunophenotypes based on the median epithelium-stroma interface immune cell density and the immune cell infiltration probability. For example, with reference to FIG. 29, the median epithelium-stroma interface immune cell density Mp may be illustrated by the dashed red line. Using the median Mp as a predictive biomarker can cause immunophenotype classifier 1710 to classify an image into one of a set of tumor immunophenotypes. For example, the set of tumor immunophenotypes may include a first tumor immunophenotype and a second tumor immunophenotype. In some embodiments, the first tumor immunophenotype may be based on the epithelium-stroma interface immune cell density being less than the median epithelium-stroma interface immune cell density (median Mp denoted by the dashed red line in FIG. 29), and the second tumor immunophenotype may be based on the epithelium-stroma interface immune cell density being greater than or equal to the median epithelium-stroma interface immune cell density. In some embodiments, the median epithelium-stroma interface immune cell density is less than 40 immune cells/mm², less than 60) immune cells/mm², less than 100 immune cells/mm², or different values. For example, the median epithelium-stroma interface immune cell density may be approximately 56 immune cells/mm².

The epithelium-stroma interface immune cell density may also be used to classify an image into one of the classical HK immunophenotypes.

In some embodiments, immunophenotype classifier 1710 may receive epithelium-stroma interface immune cell density 1332 and immune cell infiltration probability 1334 and may classify the image into one of the set of tumor immunophenotypes: desert, excluded, and inflamed. In some embodiments, immunophenotype classifier 1710 may classify an image into the desert tumor immunophenotype classification based on the epithelium-stroma interface immune cell density and the immune cell infiltration probability satisfying desert immunophenotype classification criteria. For example, immunophenotype classifier 1710 may determine that the desert immunophenotype classification criteria have been satisfied by determining that the epithelium-stroma interface immune cell density is within a first threshold range of epithelium-stroma interface immune cell densities, and the immune cell infiltration probability is within a first threshold range of immune cell infiltration probabilities. In some embodiments, the first threshold range of epithelium-stroma immune cell densities may include (i) immune cell densities less than D1 for probabilities less than probability P1, and (ii) linearly decreasing from density D1 along line T2 for probabilities greater than or equal to probability P1. The first threshold range of immune cell infiltration probabilities may include probabilities between 0% and 100%.

In some embodiments, immunophenotype classifier 1710 may classify an image into the excluded tumor immunophenotype classification based on the epithelium-stroma interface immune cell density and the immune cell infiltration probability satisfying excluded immunophenotype classification criteria. For example, immunophenotype classifier 1710 may determine that the excluded immunophenotype classification criteria have been satisfied by determining that the epithelium-stroma interface immune cell density is within a second threshold range of epithelium-stroma interface immune cell densities, and the immune cell infiltration probability being within a second threshold range of immune cell infiltration probabilities. In some embodiments, the first threshold range of epithelium-stroma interface immune cell densities may include densities greater than or equal to density D1 for probabilities less than probability P1, and densities that are less than that defined by line T1.

In some embodiments, immunophenotype classifier 1710 may classify an image into the inflamed tumor immunophenotype classification based on inflamed based on the epithelium-stroma interface immune cell density and the immune cell infiltration probability satisfying inflamed immunophenotype classification criteria. For example, immunophenotype classifier 1710 may determine that the inflamed immunophenotype classification criteria have been satisfied by determining that the epithelium-stroma interface immune cell density is within a third threshold range of epithelium-stroma interface immune cell densities, and the immune cell infiltration probabilities being within a third threshold range of immune cell infiltration probabilities. In some embodiments, the third threshold range of epithelium-stroma interface immune cell densities may include densities greater than or equal to that defined by line T1 from probabilities greater than 0).

A data point corresponding to a new image may be mapped to plot 2900. If the data point is included in group G1, then that image may be classified as representing a tumor immunophenotype of desert. If the data point is included in group G2, then that image may be classified as representing a tumor immunophenotype of excluded. If the data is included in group G3, then that image may be classified as representing a tumor immunophenotype of inflamed. Therefore, lines T1 and T2 may be used to determine an immunophenotype of an image of a tumor.

Depending on the immunophenotype, different immunotherapies and/or therapeutics may be selected. For example, certain immunotherapies may only be applicable to patients having tumors classified as inflamed. If this immunotherapy is determined to be successful in treating that patient's cancer, then it may also treat another patient effectively. To identify other patients that can be provided with this immunotherapy, patients whose tumors are also classified as being inflamed may be identified. The more patients who are recommended the successful immunotherapy, the better the overall survival of the patient cohort can be. Therefore, the three groups G1-G3 should be grouped such that the maximum number of patients who may be receptive to the effective immunotherapy are grouped into the group to be provided with the immunotherapy.

Table 1, as illustrated above, details the overall survival of patients provided with a particular immunotherapy (atezo), as well as the percentage of patients from the total population of patients in a corresponding clinical trial. In some embodiments, using the biomarkers defined by the first threshold and the second threshold may yield an OS/month of 15.6. The OS refers to a length of time from a diagnosis date or a treatment start date that the patient is still alive. For example, patients of a clinical trial associated with the data of plot 2900 may be provided the immunotherapy if those patient's tumor immunophenotype is classified as being inflamed. This classification represents approximately 34% of the total patients in the clinical trial (e.g., number of patients classified into group G3 divided by the total number of patients in the clinical trial).

In some embodiments, an epithelium-stroma interface immune cell density may alternatively be used as a biomarker for immunophenotyping. In some embodiments, a median epithelium-stroma interface immune cell density may serve as the biomarker for immunophenotyping. For example, as seen in FIG. 29, the median ESI immune cell density is represented by the dashed-red line. Based on the distributions (e.g., first distribution 2822, second distribution 2824, and portions 2832 and 2834 of the third distribution) of FIGS. 28B-28C, the median ESI immune cell density may be determined to be a density of 56 cells/mm². However, persons of ordinary skill in the art will recognize that different clinical trials may yield different median ESI immune cell densities.

In some embodiments, immunophenotype classifier 1710 may classify the image of the tumor based on the median ESI immune cell density. For example, using tumor immunophenotype classification data 1700 and the median ESI immune cell density, immunophenotype classifier 1710 may classify the image as being one of desert, excluded, or inflamed. In some embodiments, the immunophenotype may be determined on a tile-by-tile basis, and the overall immunophenotype of the image may be determined based on the immunophenotypes of the image's tiles. As an example, plot 2900 of FIG. 29 may include tumor immunophenotype classification data 2910, indicating the various groups G1-G3 for classical HK tumor immunophenotyping as well median Mp for classifying an image into the first tumor immunophenotype or the second tumor immunophenotype.

In some embodiments, immunophenotype classifier 1710 may classify an image of a tumor as being a first immunophenotype or a second immunophenotype based on the epithelium-stroma interface immune cell density computed for the image and the median epithelium-stroma interface immune cell density. For example, if the computed epithelium-stroma interface immune cell density is greater than or equal to the median epithelium-stroma interface immune cell density, immunophenotype classifier 1710 may classify the image as being of the first immunophenotype. However, if the computed epithelium-stroma interface immune cell density is less than the median epithelium-stroma interface immune cell density, immunophenotype classifier 1710 may classify the image as being the second immunophenotype.

In some embodiments, an immunotherapy may be selected for a patient based on tumor immunophenotype 1712. For example, patients whose epithelium-stroma interface immune cell density is greater than or equal to the median epithelium-stroma interface immune cell density may receive a first immunotherapy. Patients whose epithelium-stroma interface immune cell density is less than the median epithelium-stroma interface immune cell density may receive a second (different) immunotherapy. In some embodiments, the first immunotherapy may correspond to one therapeutic (e.g., azeto) and the second immunotherapy may correspond to another therapeutic (e.g., dox). In some embodiments, the first immunotherapy may correspond to a therapeutic (e.g., azeto) and the second immunotherapy may correspond to no therapeutic being provided. The median epithelium-stroma interface immune cell density, therefore, delineates between which patients will receive one immunotherapy and which patients will receive another. As seen above, with reference to Table 1, the OS/months for patients classified as being a first tumor immunophenotype (e.g., greater than or equal to the median epithelium-stroma interface immune cell density) is approximately the same as that of the HK immunophenotypes (e.g., inflamed). In other words, the overall treatment effect of the immunotherapy provided to patients classified into the first tumor immunophenotype is the same or similar to that of patients classified into the inflamed immunophenotype. However, the number of patients that are able to be grouped into the first tumor immunophenotype is larger than the number of patients that are grouped as being the inflamed immunophenotype. In other words, the number of patients who are eligible to receive the effective immunotherapy is greater than the number of patients who would otherwise be eligible based on the inflamed immunophenotype classification (e.g., 242 patients classified as being of the first tumor immunophenotype of a total number of 444 patients in the clinical trial). For example, as compared to the biomarkers delineating the immunophenotypes of desert, excluded, and inflamed, using the median epithelium-stroma interface immune cell density as the biomarker for immunotherapy selection may increase a number of patients who can be provided with the immunotherapy (e.g., 55% to 34%).

Depending on which tumor immunophenotyping is used (e.g., whether to perform classical HK tumor immunophenotyping or immunophenotyping based on the median Mp), different immunotherapies may be selected for a patient. As illustrated by Table 1, immunophenotyping based on the median epithelium-stroma interface immune cell density can allow a greater number of patients to be treated with a particular immunotherapy traditionally given to patients classified into the inflamed immunophenotype. Therefore, the use of the median epithelium-stroma interface immune cell density as a predictive biomarker for tumor immunophenotyping improves upon the classical HK tumor immunophenotype classification process by increasing the number of patients that are eligible to receive treatment traditionally reserved for patients classified into the inflamed immunophenotype.

Tumor immunophenotype classification data 1700 may represent tumor immunophenotype classifications for each of a plurality of images of tumors. For example, the images may correspond to whole slide images of a patient participating in a clinical trial for non-small cell lung cancer. A tumor immunophenotype of the tumor depicted by each image may be determined based on an epithelium-stroma interface immune cell density and an immune cell infiltration probability computed for that image. Epithelium-stroma interface image processing subsystem 116 may determine the epithelium-stroma interface immune cell density and the immune cell infiltration probability for each of a plurality of images of tumors from a plurality of patients.

Returning to FIG. 12, output generation module 350 may be configured to generate outputs corresponding to tiles 406 and/or digital pathology image 404. In some embodiments, the outputs may be generated based on a user request. As described herein, the output can include a variety of visualizations, interactive graphics, and reports based upon the type of request and the type of data that is available. In some embodiments, the output will be provided to user device 130 for display. In some embodiments, the output can be accessed directly from digital pathology image generation subsystem 110. The output may be based on existence of and access to the appropriate data. Thus, output generation module 350 may access metadata and anonymized patient information as needed. As with the other modules of epithelium-stroma interface image processing subsystem 116, output generation module 350 can be updated and improved in a modular fashion, so that new output features can be provided to users without requiring significant downtime.

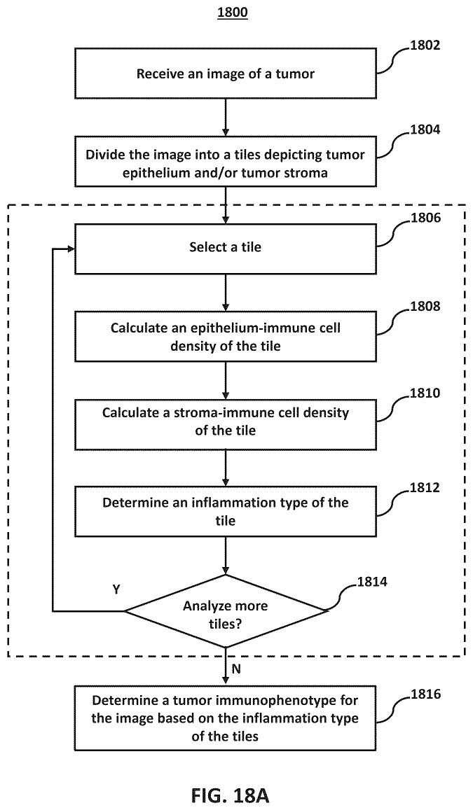

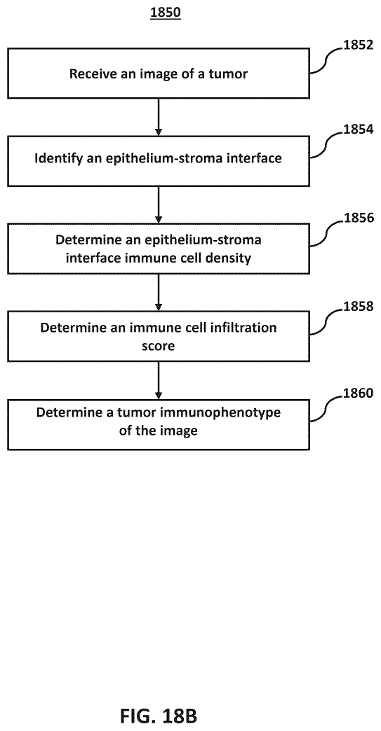

FIG. 18A illustrates an example process 1800 for determining a tumor immunophenotype, in accordance with various embodiments. Steps of process 1800 may be performed by a subsystem that is the same or similar to epithelium/stroma image processing subsystem 112. Process 1800 may begin at step 1802. At step 1802, an image of a tumor may be received. The image may be a whole slide image depicting a tissue sample of a tumor. For example, the tumor may be from a patient diagnosed with non-small cell lung cancer. In some embodiments, the patient may be part of a clinical trial whereby an immunotherapy is provided. In some embodiments, the image of the tumor may refer to a region of interest derived from a whole slide image. In some embodiments, the tumor depicted by the received image may include tumor epithelium and tumor stroma.

At step 1804, the image may be divided into a plurality of image tiles each depicting tumor epithelium, tumor stroma, or tumor epithelium and tumor stroma. The image tiles may be overlapping or non-overlapping. In some embodiments, separate image tiles depicting color channels of the image may be obtained by performing a color deconvolution process.

At step 1806, one of the tiles may be selected.

At step 1808, an epithelium-immune cell density of the selected image tile may be calculated. In some embodiments, the epithelium-immune cell density may be calculated based on a number of immune cells identified in the tumor stroma.

At step 1810, a stroma-immune cell density of the selected image tile may be calculated. In some embodiments, the stroma-immune cell density may be calculated based on a number of immune cells identified in the tumor stroma. In some embodiments, different staining agents may be used to highlight different aspects of the tumor. For example, a panCK stain may be used to highlight tumor epithelium, a CD8 stain may be used to highlight immune cells, a hematoxylin stain may be used to highlight cell nuclei, extracellular matrix, or cell cytoplasm, or other staining agents may be used. In some embodiments, the epithelium-immune cell density may be calculated using one or more image tiles highlighting immune cells in the tumor epithelium. The stroma-immune cell density may be calculated using one or more image tiles highlighting immune cells in the tumor stroma.

At step 1812, an inflammation type of the selected image tile may be determined. The inflammation type may be determined based on the stroma-immune cell density and the epithelium-immune cell density of the selected image tile. In some embodiments, the inflammation type may be a first inflammation type or a second inflammation type. In some embodiments, the inflammation type of the selected image tile may be the first inflammation type based on (i) a first stroma criterion for the stroma-immune cell density being met and (ii) a second stroma criterion for the epithelium-immune cell density being met. The inflammation type of the selected image tile may be the second inflammation type based on (iii) a first epithelium criterion for the stroma-immune cell density being met and (iv) a second epithelium criterion for the epithelium-immune cell density being met.

In some embodiments, the first stroma criterion for the stroma-immune cell density being met may include the stroma-immune cell density being greater than or equal to a stroma-immune cell density threshold. The second stroma criterion for the epithelium-immune cell density being met may include the epithelium-immune cell density being less than or equal to an epithelium-immune cell density threshold. The first epithelium criterion for the stroma-immune cell density being met may include the stroma-immune cell density being less than the stroma-immune cell density threshold. The second epithelium criterion for the epithelium-immune cell density being met may include the epithelium-immune cell density being less than the epithelium-immune cell density threshold.

In some embodiments, the stroma-immune cell density threshold and the epithelium-immune cell density threshold may be based on a number of the immune cells at an epithelium-stroma interface (ESI). In some embodiments, the stroma-immune cell density threshold and the epithelium-immune cell density threshold may be based on a number of the immune cells at the ESI divided by a total number of the tiles. In some embodiments, the stroma-immune cell density threshold and the epithelium-immune cell density threshold are based on a distribution of the immune cells, wherein the distribution is based on a plurality of distance measurements. The distance measurements may be determined by performing a color deconvolution to generate a color channel highlighting cell nuclei, identifying, based on the color channel, a plurality of immune cell nuclei, calculating the distance measurements each representing a distance from one of the immune cell nuclei to the ESI.

At step 1814, a determination may be made as to whether any additional image tiles are to be analyzed. The additional image tiles may refer to remaining image tiles from the plurality of image tiles formed by tiling the received image. In some embodiments, the additional image tiles may correspond to tiles depicting tumor epithelium and tumor stroma. If so, process 1800 may return to step 1806, where another image tile may be selected and one or more of steps 1808-1814 may be repeated. If not, however, process 1800 may proceed to step 1816. At step 1816, a tumor immunophenotype for the tumor depicted by the received image may be determined. In some embodiments, the tumor immunophenotype may be determined based on the infiltration type of the tile. Some example tumor immunophenotypes include desert, excluded, and inflamed. A tumor immunophenotype classification of desert may be determined based on a number of tiles of a first inflammation type being less than a first threshold and a number of tiles of a second inflammation type being less than a second threshold. A tumor immunophenotype classification of excluded may be based on the number of tiles of the first inflammation type being greater than or equal to the first threshold and the number of tiles of the second inflammation type being less than the second threshold. A tumor immunophenotype classification of inflamed may be based on the number of tiles of the first inflammation type being greater than or equal to the first threshold and the number of tiles of the second inflammation type being greater than or equal to the second threshold.

It is to be understood that in some embodiments, the various tumor immunophenotype classifications and inflammation types may be measured by any type of thresholds and are not limited to the comparative values listed above. For example, in some embodiments, a tumor immunophenotype classification of inflamed may be based on the number of tiles of the first inflammation type being less than the first threshold and/or the number of tiles of the second inflammation type being less than the second threshold (instead of greater than or equal to the thresholds, as described above).

FIG. 18B illustrates a flowchart of another example method 1850 for performing immunophenotyping using an epithelium-stroma interface image processing subsystem, in accordance with various embodiments. In some embodiments, method 1850) may begin at step 1852. At step 1852, an image of a tumor may be received. The image may be a whole slide image (e.g., digital pathology image 404) depicting a tissue sample of a tumor. For example, the tumor may be from a patient diagnosed with non-small cell lung cancer. In some embodiments, the patient may be part of a clinical trial whereby an immunotherapy is provided. In some embodiments, the image of the tumor may refer to a region of interest derived from a whole slide image. In some embodiments, the tumor depicted by the received image may include tumor epithelium and tumor stroma. In some embodiments, the image may be divided into a plurality of image tiles each depicting tumor epithelium, tumor stroma, or tumor epithelium and tumor stroma. The image tiles may be overlapping or non-overlapping. For example, digital pathology image 404 may be divided into tiles 406. Each tile 406 may depict tumor stroma, tumor epithelium, tumor stroma and tumor epithelium, or neither. In some embodiments, separate image tiles depicting color channels of the image may be obtained by performing a color deconvolution process.

At step 1854, an epithelium-stroma interface (ESI) may be identified. The ESI may indicate where tumor epithelium and tumor stroma separate. In some embodiments, the ESI may be determined using one or more machine learning models. For example, a computer vision model may analyze each image tile, determine whether that tile depicts tumor epithelium and tumor stroma, and if so, determine where the tumor epithelium and the tumor stroma meet. Where the tumor epithelium and the tumor stroma meet may correspond to the ESI. In some embodiments, the ESI may be identified by analyzing different color channels of a given image tile. For example, one color channel may depict an image tile that has been stained with a stain highlighting tumor epithelium whole another color channel may depict the image tile stained with a stain highlighting tumor stroma. The locations of the pixels highlighting tumor stroma can be mapped against the locations of pixels highlighting tumor epithelium to identify the ESI.

At step 1856, an epithelium-stroma interface immune cell density may be determined. The epithelium-stroma interface immune cell density may be based on the received image and may represent a number of immune cells detected within a threshold distance of the ESI. The threshold distance may be determined based on an average size of immune cells from the image. In some embodiments, immune cells may be detected by analyzing a color channel depicting an image tile stained with a stain highlighting immune cells. Based on this color channel, a location of each pixel highlighting an immune cell may be determined. These pixel locations can be compared to the pixel locations of the tumor stroma and the pixel locations of the tumor epithelium to determine a location of that immune cell with respect to the ESI. In some embodiments, the location of the pixels representing an immune cell may be used to determine an approximate center of mass of the immune cell. The pixel location of the center of mass of the immune cell may then be compared to the ESI to determine how far away the immune cell is from the ESI. If the distance is less than a distance threshold x_{Threshold}, then that immune cell may be included in the computation of the epithelium-stroma interface immune cell density.

At step 1858, an immune cell infiltration probability of immune cells may be determined. The immune cell infiltration probability may represent a probability that an immune cell in the tumor stroma will penetrate the ESI and infiltrate the tumor epithelium. In some embodiments, the immune cell infiltration probability may be determined by computing a ratio of the number of immune cells in the tumor epithelium to the number of immune cells in the tumor stroma. The greater the immune cell infiltration probability, the greater the number of immune cells that will be located within the tumor epithelium, which is desirable to improve patient mortality.

At step 1860, a tumor immunophenotype of the image may be determined. The tumor immunophenotype may be based on the epithelium-stroma interface immune cell density and the immune cell infiltration score. For example, if the classical HK tumor immunophenotype classifications are used, then the epithelium-stroma interface immune cell density and the immune cell infiltration score may be used to determine whether the image represents a tumor immunophenotype of desert, excluded, or inflamed. As another example, if the tumor immunophenotype is based on the median epithelium-stroma interface immune cell density, then the epithelium-stroma interface immune cell density for the image may be compared to the median epithelium-stroma interface immune cell density. If the computed epithelium-stroma interface immune cell density is less than the median epithelium-stroma interface immune cell density, then the image may be classified as depicting the first tumor immunophenotype, whereas if the computed epithelium-stroma interface immune cell density is greater than or equal to the median epithelium-stroma interface immune cell density, then the image may be classified as depicting the second tumor immunophenotype. Embodiments of the present disclosure further include systems, methods, devices, apparatuses, and non-transitory storage media for predicting a response to an anti-PD-L1 treatment by a patient. An exemplary system can receive an image of a tumor of the patient. Based on the image, the system can identify a plurality of immune cells in the image and an epithelium-stroma interface (ESI) separating tumor epithelium and tumor stroma in the image. The system can then calculate two features: an ESI immune cell density representing a measure of immune cells within a threshold distance of the ESI, and immune cell infiltration representing a measure of infiltration into the tumor epithelium by immune cells. The system can then predict the response to the anti-PD-L1 treatment by inputting the ESI immune cell density and the immune cell infiltration into a model such as a machine-learning model.

In an exemplary implementation, an automated method is implemented for quantifying the concentration and infiltration of CD8+ T cells at the tumor boundary and it is demonstrated that these features relate to patient overall survival (OS) after atezolizumab treatment in non-small cell lung cancer (NSCLC). Specifically, digital pathology is used to generate a digital assessment of cytotoxic T-cell infiltration (DACTI). DACTI identifies a subset of patients who benefited from atezolizumab in an independent validation set drawn from a randomized phase III trial of atezolizumab vs, docetaxel in advanced NSCLC. This is true even within the PD-L1-negative sub-cohort, a group which has exhibited uneven treatment benefit from atezolizumab. Accordingly, automated measurement of CD8+ T-cell distribution at the tumor epithelial-stromal interface is shown to be associated with atezolizumab benefit and transcriptomic pathways in NSCLC.

Embodiments of the present disclosure provide several technical advantages. For example, some embodiments described herein are distinguished from existing solutions by specific measurement of cytotoxic T-cells, analysis of immune cell infiltration patterns, and validation on a large independent patient set drawn from a randomized clinical trial. Further, bulk RNA-seq from matched samples is used to confirm this digital pathology approach captured features associated with CD8 T cell infiltration. Embodiments of the present disclosure are highly interpretable due to the use of specific, hypothesis-driven features of immune cell distribution. This is in contrast to purely deep learning approaches, which have been implemented in the computational pathology space but are largely unexplainable due to being “black boxes” in which it is a challenge to relate specific tissue morphologies to the machine's predictions. Such lack of transparency has been identified as a detrimental factor for clinician trust in machine learning approaches. Further, manual measurement of the distance of every T cell from the ESI would be impractically tedious and time-consuming. In embodiments of the present disclosure, the use of computer vision and machine learning approaches to quantify tissue appearance offers a high-throughput, reproducible method for measuring cell positions.

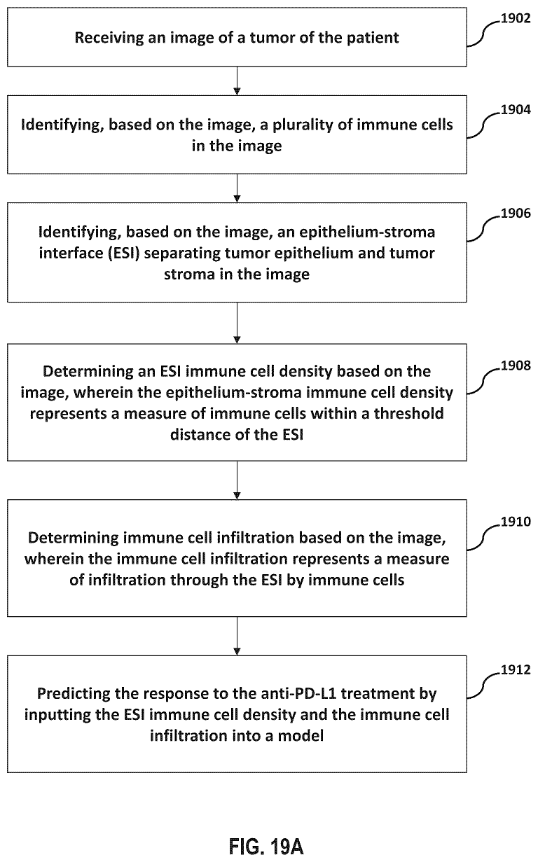

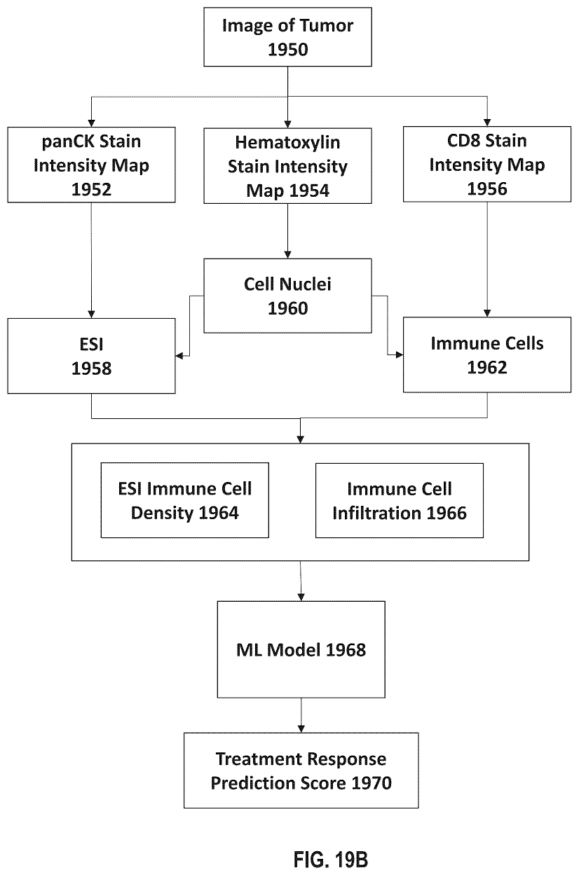

FIG. 19 illustrates an exemplary method 1900 for predicting a response to an anti-PD-L1 treatment by a patient. Anti-PD-L1 drugs include a type of immunotherapy used in cancer treatment that can work by targeting the programmed death-ligand 1 (PD-L1) protein, which cancer cells can use to evade detection by the immune system. By blocking PD-L1, these drugs help the immune system recognize and attack cancer cells. In some examples, the anti-PD-L1 treatment comprises: atezolizumab, avelumab, or durvalumab.

At block 1902, an exemplary system (e.g., one or more electronic devices) receives an image of a tumor (e.g., solid tumor) of the patient. The image can be any type of image as described herein. In some embodiments, a tissue sample is obtained from the patient and the tissue sample is stained with one or more dyes. For example, the tissue sample may be stained with a stain that distinguishing between the tumor stroma and the tumor epithelium, such as a pan-cytokeratin (panCK) stain highlighting the epithelial cells. The tissue sample may be additionally stained with a stain that marks immune cells, such as a CD8 stain highlighting the CD8 cells. The tissue sample can be further stained with hematoxylin. The panCK/CD8 slide can be digitized using an imager, such as a whole-slide scanner, to generate the image. In some embodiments, the system performs one or more preprocessing operations on the image, such as removal of artifacts and necrosis. In some embodiments, the system identifies the tumor lesion area (e.g., based on a tumor lesion detection algorithm, based on user annotations) and exclude the non-tumor area from further analysis described below.

To measure the spatial distribution of immune cells (e.g., cytotoxic T cells) with regard to the ESI, the system first identifies the immune cells as well as the epithelial and stromal regions. In some embodiments, the system performs color deconvolution on the image (or the tumor lesion region depicted in the image) to digitally separate three color channels corresponding to the various stains (e.g., the hematoxylin stain, the panCK stain, and the CD8 stain). For example, for the image, the system can obtain a stain intensity map for each of the three stains in the image: a hematoxylin stain map, a panCK stain map, and a CD8 stain map.

Based on the hematoxylin stain map, the system can perform cell segmentation to identify a plurality of cell nuclei in the image. In some embodiments, the system can perform cell segmentation using a machine-learning model, such as Cellpose, to automatically identify and delineate individual cells in the image. In some embodiments, prior to segmentation, contrast-limited adaptive histogram equalization can be applied to the hematoxylin stain map to increase image contrast, improving segmentation performance.

For each of the panCK and CD8 stain maps, the system can apply an intensity threshold to produce a panCK stain binary mask and a CD8 stain binary mask for the image. The panCK stain binary mask can comprise pixel-wise binary values, with the value 1 indicative of the presence of panCK stain (i.e., marking the epithelial cells) and the value 0 indicative of the absence of panCK stain. Similarly, CD8 stain binary mask can comprise pixel-wise binary values, with the value 1 indicative of the presence of CD8 stain (i.e., marking the CD8 T cells) and the value 0 indicative of the absence of CD8 stain. In some embodiments, these binary masks can be further processed to smooth edges and remove small objects and small holes (e.g., based on one or more size thresholds).

At block 1904, the system identifies, based on the image, a plurality of immune cells in the image. As discussed above, based on the hematoxylin stain map, the system can perform cell segmentation to identify a plurality of cell nuclei in the image. Further, the system can determine whether each cell is an immune cell by determining whether the location of the cell nuclei intersects with the CD8 stain regions as indicated in the CD8 binary mask or the CD8 stain map. Accordingly, each cell nuclei can be labelled as an immune cell or not an immune cell. In some embodiments, all nuclei within necrotic or artifact regions (e.g., identified by user annotations, identified by artifact or necrosis detection algorithms) are excluded from further analysis.

At block 1906, the system identifies, based on the image, an epithelium-stroma interface (ESI) separating tumor epithelium and tumor stroma in the image. In some embodiments, the system first identifies the tumor stroma area in the image. Identification of the tumor stroma area can include two steps because there is no cytoplasmic staining of stromal cells. First, the system identifies a first group of pixels in the image based on a luminosity threshold by comparing the luminosity threshold with each pixel of the image to identify pixels that are darker than the luminosity threshold. The identified pixels represent tissue, which is darker than the background. All pixels that are identified as tissue and are also panCK-negative (based on the panCK stain binary mask) can be labeled as stroma. In this first step, the identified area may not capture the entirety of the stroma area due to the transparency of the cytoplasm of panCK-negative cells. Thus, in a second step, the system dilates all segmented nuclei in the panCK-negative regions by five microns and include the additional area as part of the tumor stroma region. In other words, the union of the results of the first and second steps can form the tumor stroma area. The tumor epithelium area in the image can be identified as the panCK-positive pixels. The ESI can be then identified as the boundary between the tumor stroma area and the tumor epithelium area in the image.

After the ESI is identified, the system can calculate, for each cell in the image, a distance between the cell and the ESI. Each cell nucleus (e.g., as identified by the hematoxylin stain map) can be labelled as either epithelial (if its boundary intersects the panCK region indicated by the panCK stain map) or stromal (if it does not). For a nucleus labeled as stromal, the cell-ESI distance can be calculated as the distance between the nucleus centroid and the nearest epithelial area. For a nucleus labeled as epithelial, the cell-ESI distance can be calculated as the distance between the nucleus centroid and the nearest stromal area. The nearest opposite-type tissue region is used instead of the boundary of the tissue region containing the nucleus to avoid counting tissue edges or lumens as ESI locations.

At block 1910, the system determines an ESI immune cell density based on the image. The epithelium-stroma immune cell density represents a measure of immune cells within a threshold distance of the ESI. In some embodiments, the ESI immune cell density comprises a ratio of a number of immune cells within the threshold distance of the ESI and a total number of cells within the threshold distance of the ESI. For example, the system counts the immune cells (identified in block 1904) that are within the threshold distance of the ESI (identified in block 1906), counts all cells (identified based on the hematoxylin stain map) that are within the threshold distance of the ESI, and calculates the ratio. The threshold distance can be determined based on the size of a cell. In some embodiments, the threshold distance is 8 microns. A higher density can be indicative of greater immune cell attraction to the tumor.

At block 1912, the system determines immune cell infiltration based on the image. The immune cell infiltration represents a measure of infiltration into the tumor epithelium by immune cells. In some embodiments, the immune cell infiltration comprises a ratio between a first ratio and a second ratio, with the first ratio being between a number of immune cells and a total number of cells within a band area in the tumor epithelium, and the second ratio being between a number of immune cells and a total number of cells within a band area in the tumor stroma. The band area in the tumor epithelium can be the area between a first distance and a second distance from the ESI in the tumor epithelium, and the band area in the tumor stroma can be the area between the first distance and the second distance from the ESI in the tumor stroma. For example, the system can calculate the fraction of immune cells over all cells in the epithelium within 8 and 24 microns from the ESI, divided by the fraction in the corresponding band in the stroma. Alternatively, the immune cell infiltration comprises a ratio between a number of immune cells within the band in the tumor epithelium and a number of immune cells within the band in the tumor stroma. A higher immune cell infiltration may be indicative of greater immune cell diffusion into the tumor epithelium. Calculating the immune cell infiltration based on the ratio of two ratios, rather than the ratio of counts, can eliminate additional confounders or correlations with the ESI immune cell density calculated in block 1910 and thus can produce a more accurate model.

At block 1914, the system predicts the response to the anti-PD-L1 treatment by inputting the ESI immune cell density and the immune cell infiltration into a trained model. The model can comprise a machine-learning model or a statistical model.

To train the machine-learning model, the system obtains a set of training images obtained from a cohort of patients that have been treated with the anti-PD-L1 treatment. For each image, the ESI immune cell density and the immune cell infiltration are calculated. The system can then construct a training dataset comprising, for each training image, the ESI immune cell density, the immune cell infiltration, and the treatment response by the patient from which the image is obtained (e.g., survival). In some embodiments, the treatment response for each patient in the training dataset can be a binary value (e.g., indicative of whether the patient responded to the treatment), an integer (e.g., indicative of how much the patient responded to the treatment), a continuous value, a percentage, a percentile, a classification (e.g., high, medium, or low response), or any combination thereof. The training dataset can be then used to train the model. During training, the model receives, for each training image, the ESI immune cell density and the immune cell infiltration and outputs a prediction. The prediction is then compared against the ground-truth treatment response and the model (e.g., weights of the model) can be updated based on the comparison. In some embodiments, the model can be retrained iteratively over time using updated training datasets.

For example, a Cox proportional hazards model is fitted using the training dataset to predict a treatment response score, also referred to as cytotoxic T-cell infiltration (DACTI). Specifically, the model is fitted by fitting coefficients β1 and β2 in:

$$DACTI = {β}_{1} {T}_{ESI} + {β}_{2} {T}_{inf}$$

After the coefficients are fitted, the system can apply the model to a cohort of patients who have been treated with the anti-PD-L1 treatment to calculate each patient's treatment response score. Based on the treatment response scores and the patients' outcomes, the system can identify a threshold. For example, the threshold can be identified to maximize the difference in median survival time between the anti-PD-L1 treatment and the non-anti-PD-L1 treatment arms in patients above the threshold.

The model can comprise a supervised model, an unsupervised model, a semi-supervised model, a self-supervised model, an ensemble model, a deep learning model, or any combination thereof. In some embodiments, the predicted first or second treatment response comprises: a probability value, a binary value, an integer, a classification, or any combination thereof. For example, the model can be a Linear Discriminant Analysis (LDA) model, a Quadratic Discriminant Analysis (QDA) model, or a K-Nearest Neighbor model.

In some embodiments, the model can be configured to receive additional features, such immune cell density in the tumor lesion overall, immune cell density in the intra-tumoral stroma, and immune cell density in the tumor epithelium, etc.

In some embodiments, at block 1914, the system can obtain a treatment response prediction score from the model and compare the treatment response against the threshold. For example, if the score exceeds the threshold, the system can identify the patient as DACTI-high, or otherwise DACTI-low. The system can then determine a treatment recommendation. For example, the system can recommend the anti-PD-L1 treatment if the patient is classified as DACTI-high.

FIG. 19B illustrates an exemplary method for predicting a response to an anti-PD-L1 treatment by a patient. An exemplary system (e.g., one or more electronic devices) receives an image 1950 of a tumor (e.g., solid tumor) of the patient. The image can be any type of image as described herein. In some embodiments, a tissue sample is obtained from the patient and the tissue sample is stained with one or more dyes. For example, the tissue sample may be stained with a stain that distinguishing between the tumor stroma and the tumor epithelium, such as a pan-cytokeratin (panCK) stain highlighting the epithelial cells. The tissue sample may be additionally stained with a stain that marks immune cells, such as a CD8 stain highlighting the CD8 cells. The tissue sample can be further stained with hematoxylin. The panCK/CD8 slide can be digitized using an imager, such as a whole-slide scanner, to generate the image. In some embodiments, the system performs one or more preprocessing operations on the image, such as removal of artifacts and necrosis. In some embodiments, the system identifies the tumor lesion area (e.g., based on a tumor lesion detection algorithm, based on user annotations) and exclude the non-tumor area from further analysis described below.

The system performs color deconvolution on the image (or the tumor lesion region depicted in the image) to digitally separate three color channels corresponding to the various stains (e.g., the hematoxylin stain, the panCK stain, and the CD8 stain). In the depicted example, for the image 1950, the system can obtain a stain intensity map for each of the three stains in the image: a hematoxylin stain map 1954, a panCK stain map 1952, and a CD8 stain map 1956.

Based on the hematoxylin stain map 1960, the system can perform cell segmentation to identify a plurality of cell nuclei 1960 in the image. In some embodiments, the system can perform cell segmentation using a machine-learning model, such as Cellpose, to automatically identify and delineate individual cells in the image. In some embodiments, prior to segmentation, contrast-limited adaptive histogram equalization can be applied to the hematoxylin stain map to increase image contrast, improving segmentation performance.

The system identifies, based on the image, a plurality of immune cells 1962 in the image. As discussed above, based on the hematoxylin stain map, the system can perform cell segmentation to identify a plurality of cell nuclei 1960 in the image. Further, the system can determine whether each cell is an immune cell by determining whether the location of the cell nuclei intersects with the CD8 stain regions based on the CD8 stain map 1962. Accordingly, each cell nuclei can be labelled as an immune cell or not an immune cell. In some embodiments, all nuclei within necrotic or artifact regions (e.g., identified by user annotations, identified by artifact or necrosis detection algorithms) are excluded from further analysis.

The system identifies, based on the image, an ESI 1958 separating tumor epithelium and tumor stroma in the image. In some embodiments, the system first identifies the tumor stroma area in the image. Identification of the tumor stroma area can include two steps because there is no cytoplasmic staining of stromal cells. First, the system identifies a first group of pixels in the image based on a luminosity threshold by comparing the luminosity threshold with each pixel of the image to identify pixels that are darker than the luminosity threshold. The identified pixels represent tissue, which is darker than the background. All pixels that are identified as tissue and are also panCK-negative (based on the panCK stain map 1952) can be labeled as stroma. In this first step, the identified area may not capture the entirety of the stroma area due to the transparency of the cytoplasm of panCK-negative cells. Thus, in a second step, the system dilates all segmented nuclei in the panCK-negative regions by five microns and include the additional area as part of the tumor stroma region. In other words, the union of the results of the first and second steps can form the tumor stroma area. The tumor epithelium area in the image can be identified as the panCK-positive pixels. The ESI can be then identified as the boundary between the tumor stroma area and the tumor epithelium area in the image.

After the ESI is identified, the system can calculate, for each cell in the image, a distance between the cell and the ESI. Each cell nucleus (e.g., as identified by the hematoxylin stain map) can be labelled as either epithelial (if its boundary intersects the panCK region indicated by the panCK stain map) or stromal (if it does not). For a nucleus labeled as stromal, the cell-ESI distance can be calculated as the distance between the nucleus centroid and the nearest epithelial area. For a nucleus labeled as epithelial, the cell-ESI distance can be calculated as the distance between the nucleus centroid and the nearest stromal area. The nearest opposite-type tissue region is used instead of the boundary of the tissue region containing the nucleus to avoid counting tissue edges or lumens as ESI locations.

The system determines an ESI immune cell density 1964 based on the image. In some embodiments, the ESI immune cell density comprises a ratio of a number of immune cells within the threshold distance of the ESI and a total number of cells within the threshold distance of the ESI. For example, the system counts the immune cells 1962 that are within the threshold distance of the ESI 1958, counts all cells 1960 that are within the threshold distance of the ESI 1958, and calculates the ratio. The threshold distance can be determined based on the size of a cell. In some embodiments, the threshold distance is 8 microns. A higher density can be indicative of greater immune cell attraction to the tumor.

The system determines immune cell infiltration 1966 based on the image. In some embodiments, the immune cell infiltration comprises a ratio between a first ratio and a second ratio, with the first ratio being between a number of immune cells and a total number of cells within a band in the tumor epithelium, and the second ratio being between a number of immune cells and a total number of cells within a band in the tumor stroma. The band in the tumor epithelium can be the area between a first distance and a second distance from the ESI in the tumor epithelium, and the band in the tumor stroma can be the area between the first distance and the second distance from the ESI in the tumor stroma. For example, the system can calculate the fraction of immune cells over all cells in the epithelium within 8 and 24 microns from the ESI, divided by the fraction in the corresponding band in the stroma. Alternatively, the immune cell infiltration comprises a ratio between a number of immune cells within the band in the tumor epithelium and a number of immune cells within the band in the tumor stroma. A higher immune cell infiltration may be indicative of greater immune cell diffusion into the tumor epithelium. Calculating the immune cell infiltration based on the ratio of two ratios, rather than the ratio of counts, can eliminate additional confounders or correlations with the ESI immune cell density 1964 and thus can produce a more accurate model.

The system predicts the response to the anti-PD-L1 treatment by inputting the ESI immune cell density 1964 and the immune cell infiltration 1966 into a model 1958. The model can comprise a machine-learning model or a statistical model. As described herein, the model can comprise a supervised model, an unsupervised model, a semi-supervised model, a self-supervised model, an ensemble model, a deep learning model, or any combination thereof, the system can obtain a treatment response prediction score from the model and compare the treatment response against the threshold. For example, if the score exceeds the threshold, the system can identify the patient as DACTI-high, or otherwise DACTI-low. The system can then determine a treatment recommendation. For example, the system can recommend the anti-PD-L1 treatment if the patient is classified as DACTI-high.

In some embodiments, the predicted treatment responses may be used for companion diagnostic tests, for example, to select patients for future trials using atezolizumab in combination with other immune checkpoint inhibitors. In some embodiments, the predicted treatment responses may be used for the expansion of treatment eligibility to PD-L1-negative patients not currently considered candidates for atezolizumab.

### Example

This example describes exemplary techniques that can be used for extracting various features described herein and training machine-learning models. Although particular methods and protocols are provided, one skilled in the art will appreciate that variations can be made. In this example, a method is implemented for automated quantification of the density and epithelial infiltration of cytotoxic T cells at the tumor epithelial-stromal interface (ESI). The machine learning model was trained to generate a digital assessment of cytotoxic T-cell infiltration (DACTI), on n=188 patients and validated the association of DACTI with atezolizumab benefit on n=833 patients from a randomized phase III trial of atezolizumab vs. docetaxel in non-small cell lung cancer. A higher density of CD8+ T cells at the ESI and greater infiltration of those cells into the tumor epithelium were associated with atezolizumab benefit. These imaging-based phenotypic markers were also correlated with bulk RNAseq expression of genes related to a variety of immune cell types. Atezolizumab-treated patients had a longer overall survival (OS) than docetaxel-treated patients in the DACTI-high patients of the validation set (n=270, hazard ratio [HR]=0).65, 95% confidence interval (CI): 0.49-0.88, treatment interaction p-value=0.49). No difference between arms was observed in the DACTI-low group (HR=0.95, 95% CI: 0.77-1.13). These results suggest that the mechanisms driving response to atezolizumab can potentially be uncovered by digital image analysis, which may have utility in companion diagnostics.

### Dataset

A dataset was drawn from two clinical trials comparing docetaxel to atezolizumab (anti-PD-L1) as single-agent second-line therapy in advanced-stage NSCLC. These trials enrolled patients diagnosed with adenocarcinoma or squamous carcinoma who had experienced progression after platinum chemotherapy.

For each patient, a tissue sample was obtained and the tissue section was stained with pan-cytokeratin (panCK), which marked the epithelial cells, and CD8 in a dual chromogenic IHC assay. A single panCK/CD8 slide was digitized for each patient using a DP200 whole-slide scanner at a resolution of 0.24 micrometers-per-pixel, approximately equivalent to 40× magnification. Following digitization, the tumor lesion area was annotated on each slide by a board-certified anatomic pathologist using an internal digital pathology viewer and annotation tool.

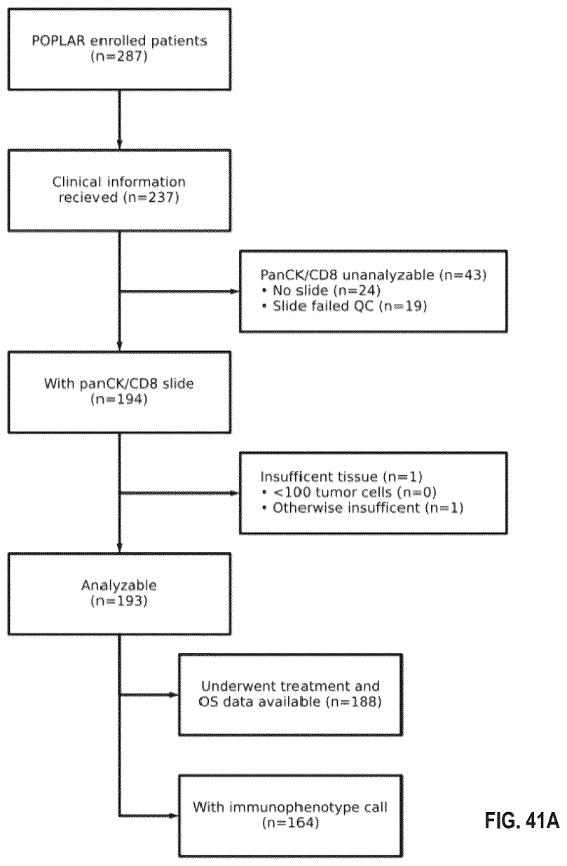

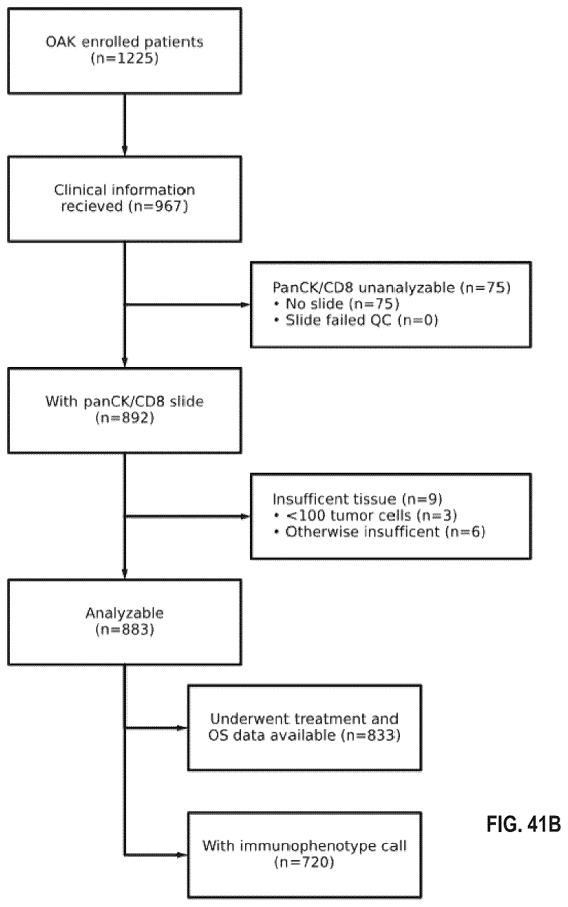

FIGS. 41A-B illustrate flow diagrams for patient enrollment in the study for the training set, drawn from the POPLAR trial and the validation set drawn from the OAK trial. Among the successfully digital panCK/CD8 slides, only those with at least 100 viable tumor cells visible are selected for further processing and analysis. POPLAR, a phase II study, provided 193 patients whose images met these criteria. The follow-on phase III study, OAK, provided 883 patients whose images met these criteria. Accordingly, a total study dataset of 1076 patients was obtained. Further, using the annotations provided by the pathologist, non-tumor areas as well as areas with necrosis or artifacts were excluded from analysis.

As described further below, patients from POPLAR were used to train the atezolizumab-benefit machine-learning model, and patients from OAK were used as the validation set for the trained machine-learning model. The split of the training and validation sets was made on the basis of trial to ensure the two sets were entirely independent, with the larger trial (i.e., OAK) used as the validation set to maximize the confidence in model performance metrics.

### Stain Separation and Compartment Segmentation

In order to measure the spatial distribution of cytotoxic T cells with regard to the ESI, a system analyzed each image to identify the CD8+ cells as well as the epithelial and stromal regions. First, the system applied color deconvolution within the pathologist-annotated tumor lesion to digitally separate the hematoxylin, panCK, and CD8 stains. The stain optical density matrix used for color deconvolution was initialized by an automated method, for example, as described in Macenko et al., Macenko M, Niethammer M, Marron J S, Borland D, Woosley J T, Guan X, et al. A METHOD FOR NORMALIZING HISTOLOGY SLIDES FOR QUANTITATIVE ANALYSIS. 2009 IEEE Int Symp Biomed Imaging: Nano Macro. 2009; 1107-10, the content of which is incorporated herein in its entirety. The stain optical density matrix was then manually refined to produce the best qualitative color separation and then used for stain separation in all the images.

For each image, color deconvolution produced a stain intensity map for each of the three stains in that image: a hematoxylin stain map, a panCK stain map, and a CD8 stain map. For each of the panCK and CD8 stain maps, the system applied an intensity threshold to produce a panCK stain binary mask (which indicates the location of panCK stain in the image) and a CD8 stain binary mask (which indicates the location of CD8 stain in the image) for each image. These binary masks were further processed to smooth edges and remove small objects and small holes (e.g., based on one or more size thresholds).

Identification of stroma included two steps because there was no cytoplasmic staining of stromal cells. First, the system compared a luminosity threshold with each pixel of the image to identify pixels that are darker than the luminosity threshold. The identified pixels represented tissue, which was darker than the background. All pixels that are identified as tissue and are panCK-negative (based on the panCK stain binary mask) were labeled as stroma. In the first step, the identified area did not capture the entirety of the stroma area due to the transparency of the cytoplasms of panCK-negative cells. Thus, in a second step, the segmentations of all nuclei in the panCK-negative regions were dilated by five microns. The union of the results of the first and second steps, with panCK regions subtracted out, formed the stromal region label.

### Nucleus Segmentation and Labeling

In each image, nuclei were segmented based on the hematoxylin stain map using the publicly available Cellpose method with the pretrained cyto2 model. Prior to segmentation, contrast-limited adaptive histogram equalization was applied to the hematoxylin stain map to increase image contrast, improving segmentation performance. Each nucleus was then labeled either epithelial (if its boundary intersected the panCK region indicated by the panCK stain map) or stromal (if it did not).

Further, each nucleus was labeled for CD8 positivity according to intersection with the CD8 regions (as indicated by the CD8 stain map). All nuclei within pathologist-annotated necrotic or artifact regions were removed.

The system calculated a cell-ESI distance for each cell. For a nucleus labeled as stromal, the cell-ESI distance was calculated as the distance between the nucleus centroid and the nearest epithelial region. For a nucleus labeled as epithelial, the cell-ESI distance was calculated as the distance between the nucleus centroid and the nearest stromal region. The nearest opposite-type tissue region was used instead of the boundary of the tissue region containing the nucleus to avoid counting tissue edges or lumens as ESI locations.

### Immune Tumor Spatial Analysis Feature Extraction

Two features were extracted for the purpose of quantifying the TME. The first feature quantifies T-cell involvement with the tumor as the density of T-cells at the ESI (T_{ESI}). In this example, the first feature was defined as the fraction of cells within eight microns of the ESI which were CD8+. A higher T_{ESI} value would be associated with greater T cell attraction to the tumor.

The second feature quantifies the infiltration of T-cells into the tumor epithelium (T_{inf}) as the ratio of cells close to the ESI in epithelium to that in the stroma. In this example, the second feature was defined as the fraction of cells in the epithelium within 8 and 24 microns of the ESI which were CD8+, divided by the fraction in the corresponding band in stroma. A higher T_{inf} would be associated with greater T cell diffusion into the tumor epithelium.

Three additional features were also extracted for comparison purposes. The three additional features include T_{dens(overal)}, which measures the CD8+ cell density in the tumor lesion overall, and T_{dens(stroma)}, which measures the CD8+ cell density in the intra-tumoral stroma, and T_{dens(epi)}, which measures the CD8+ cell density in the tumor epithelium. Previous literature indicates that lymphocyte density in the intra-tumoral stroma may be a predictive marker for ICI in NSCLC. To verify that the novel features T_{ESI} and T_{inf} add new predictive information rather than recapitulating lymphocyte density, the predictive power of each of the three T_{dens} features was also tested. A substantial performance difference between the T_{dens} descriptors and the novel features would indicate that the novel features contained information orthogonal to existing measures of T-cell density.

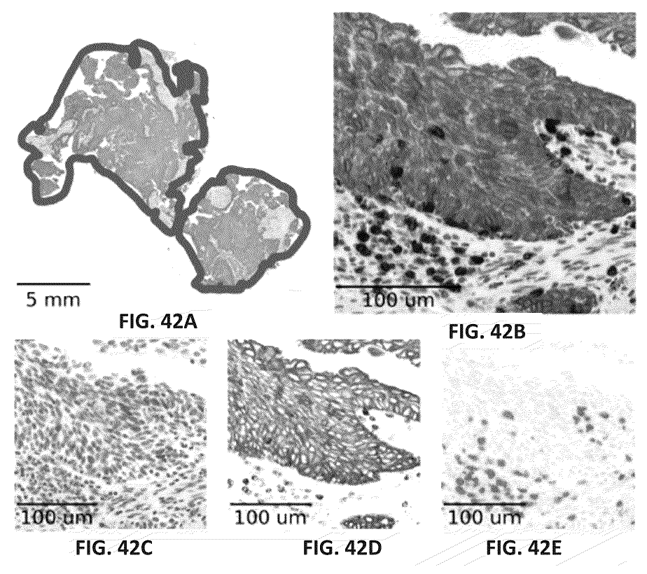

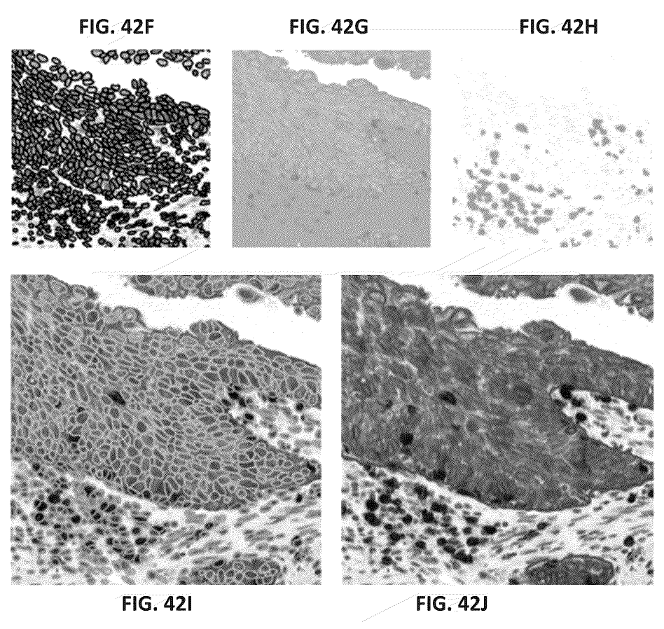

FIGS. 42A-J provide exemplary visualization of the DP analysis process, in accordance with some embodiments. FIG. 42A depicts pathologist tumor lesion annotations on a panCK/CD8-stained slide. FIG. 42B depicts a high-magnification ROI for visualization. Color deconvolution results are shown for the hematoxylin channel (FIG. 42C), panCK channel (FIG. 42D), and CD8 channel (FIG. 42E). Those channels are used for nuclear segmentation (FIG. 42F), defining the epithelial and stromal regions or areas (FIG. 42G), and identifying CD8+ cells (FIG. 42J). Features are extracted using the final labeled nuclei (FIG. 421) and the ESI (FIG. 42J).

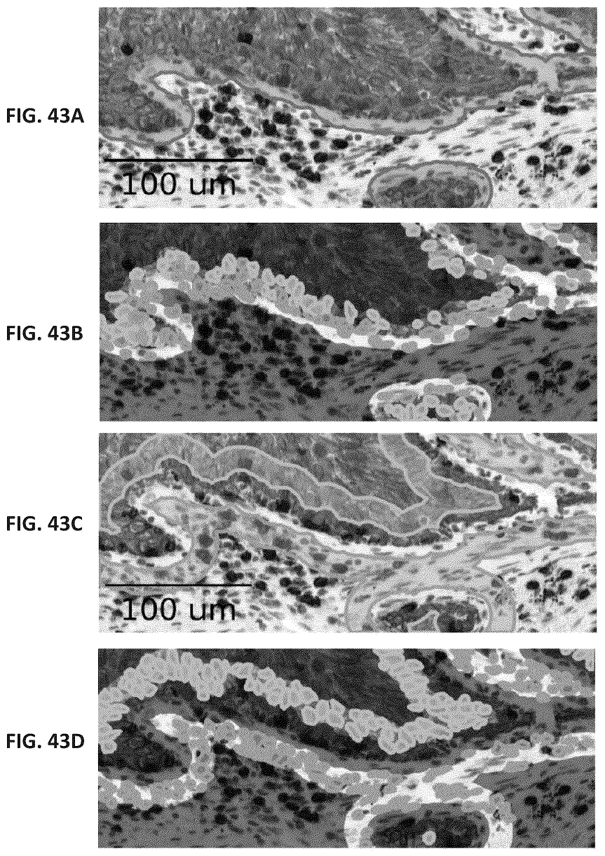

FIGS. 43A-D provide exemplary visualization of the ESI immune cell density T_{ESI} (FIGS. 43A-B) and the immune cell infiltration T_{inf} (FIGS. 43C-D) in a sample region of interest. FIG. 43A depicts the area within 8 μm of the ESI and FIG. 43B depicts nuclei within 8 μm of the ESI. T_{ESI} is equal to the fraction of these nuclei which are CD8+. FIG. 43C depicts the area between 8 and 24 μm from the ESI. FIG. 43D depicts nuclei 8 to 24 μm from the ESI. T_{inf} is equal to the fraction of nuclei in this band in the epithelial compartment which are CD8+ divided by that measure in the corresponding band in the stroma.

### Experiment 1: Characterization of the Tumor Immune Environment

The features T_{ESI} and T_{inf} characterized two separate characteristics of CD8+ T-cell distribution: concentration (T_{ESI}) and infiltration (T_{inf}). This was assessed by measuring the degree to which these two features separated pathologist-called immune desert, excluded, and inflamed patients as well as the independence of the two features from each other. The correlation between each pair of the features was also measured by Spearman correlation coefficient (SCC). While these features were not developed with the intention of recapitulating pathologist scoring, they were inspired by the approach of pathologists in categorizing patients by T-cell concentration and infiltration. It was therefore expected these features to be somewhat correlated with pathologist immunophenotyping calls. Separation of pathologist-called immunophenotypes was assessed by the top-1 accuracy and macro-weighted F1 score of a quadratic discriminant analysis (QDA) classifier trained on T_{ESI} and T_{inf} in the training set and tested in the validation set. A high accuracy would indicate that the features could predict pathologist labels, and therefore that the features were associated with characteristics of the immune morphology also apparent to pathologists.

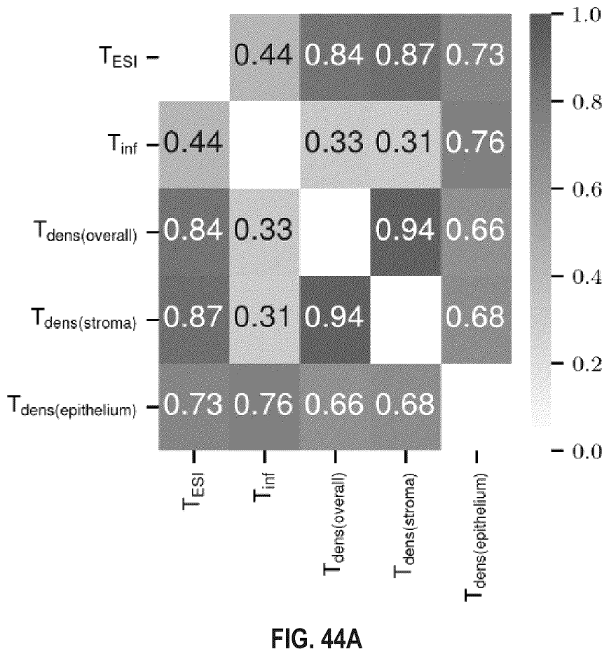

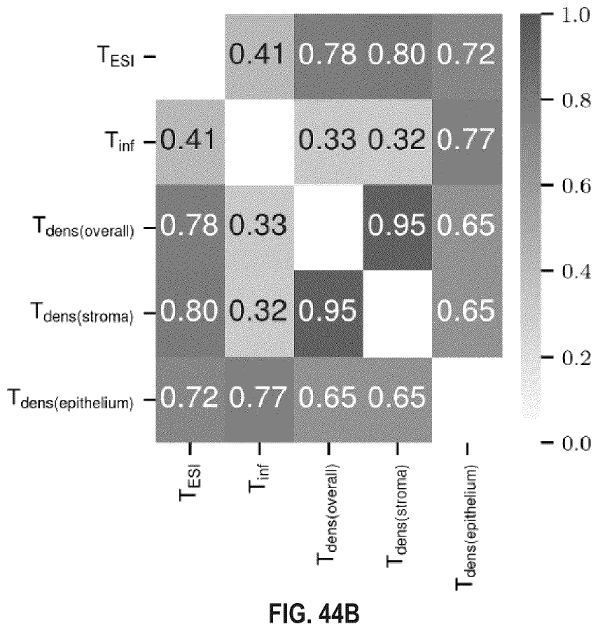

FIGS. 44A-B illustrate the Spearman correlation coefficients between T_{ESI}, T_{inf}, and measures of CD8+ cell density in the tumor overall, in the intra-tumoral stroma, and in the tumor epithelium in the training set (FIG. 44A) and the validation set (FIG. 44B), respectively. FIGS. 44A-B show that T_{ESI} and T_{inf} describe independent aspects of the tumor immune landscape. As shown, T_{ESI} and T_{Inf} were moderately correlated, with an SCC of 0.44 in the training set and 0.41 in the validation set. While T_{ESI} was strongly correlated with the density of CD8+ T cells in the intra-tumoral stroma and tumor epithelium in the validation set (SCC=0.78, 0.80 respectively), T_{inf} was not (SCC=0.33, SCC=0.32). Both measures were correlated with overall CD8+ T cell density in the tumor (SCC=0.72, SCC=0.77). Overall, T_{ESI} and T_{Inf} appear to measure somewhat independent aspects of the immune spatial landscape and provide insight beyond what is captured by CD8+ T cell density alone.

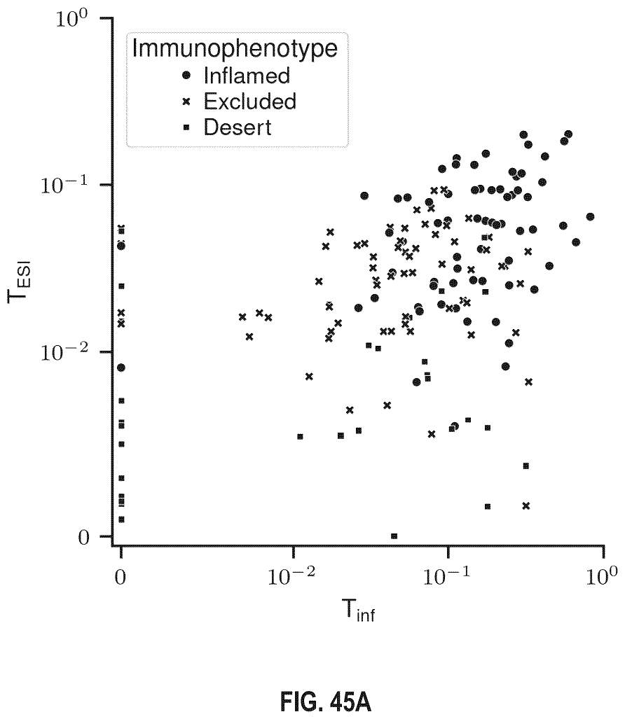

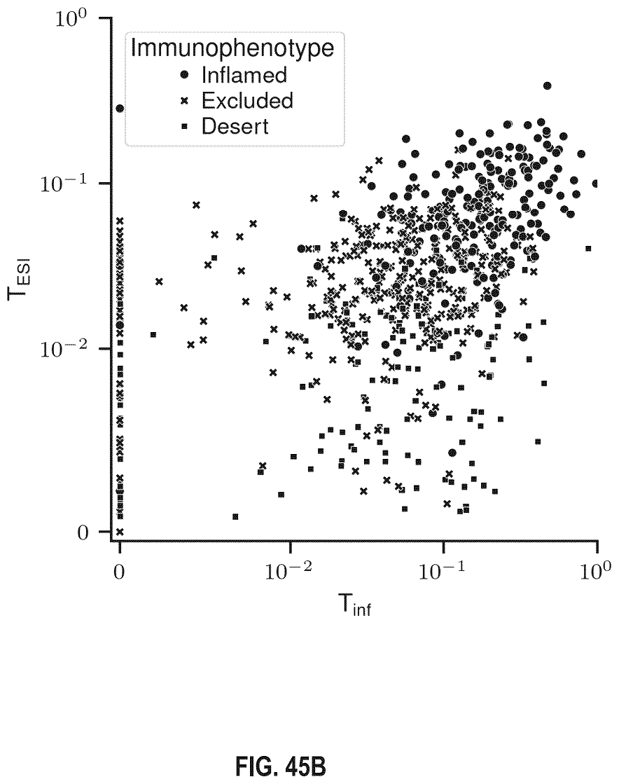

FIGS. 45A-B illustrate the distribution of patient T_{ESI} and T_{inf} values in the n=164 training set patients (FIG. 45A) and n=720 validation set patients who had a valid pathologist immunophenotype call available (FIG. 45B). Patients are colored according to the assigned immunophenotype. The QDA model fit on the training set to predict immunophenotype had a macro-weighted F1 score of 0.62 and top-1 accuracy of 0.63 on the validation set. Qualitatively, clusters of immunophenotypes are evident in the scatterplot of T_{ESI} and T_{Inf} as shown in the figures. Inflamed cases tended to have a high T_{ESI} and T_{Inf}, excluded cases had high T_{ESI} and low T_{inf}, and desert cases had low T_{ESI}.

### Experiment 2: Correlation of Digital Pathology Features with Bulk RNAseq Signatures

To understand the biology captured by T_{ESI} and T_{inf}, feature values were compared to bulk RNA-sequencing from the same tissue blocks. Each feature was separately used in gene set enrichment analysis (GSEA) using these matched RNA-seq transcriptomes. For this analysis, the training and testing sets were combined into a single dataset of n=709 patients. Patients were then stratified as being above or below median the T_{ESI} and T_{inf} values, and signatures were selected for a broad variety of TME cell types, for example as described in Bagaev et al., Conserved pan-cancer microenvironment subtypes predict response to immunotherapy. Cancer Cell. 2021 Jun. 14; 39(6):845-865.e7. doi: 10.1016/j.ccell.2021.04.014. Epub 2021 May 20. PMID: 34019806, the content of which is incorporated herein by reference. GSEA was carried out using the qusage R package (v2.34.0) in R v4.3.1, for example, as described in Yaari et al., Quantitative set analysis for gene expression: a method to quantify gene set differential expression including gene-gene correlations, Nucleic Acids Res. 2013 October; 41 (18):e170, doi: 10.1093/nar/gkt660. Epub 2013 Aug. 5. PMID: 23921631; PMCID: PMC3794608, the content of which is incorporated herein by reference. Significant associations between the digital pathology features and tumor RNA expression were then identified using the p-values (calculated using the probability density function for each gene set vs, the null hypothesis of no fold change as part of the qusage function) for each signature across the above/below median groups of patients. Histology and resection status were used as covariates in the GSEA, due to their known differential transcriptional differences.

T_{ESI} and T_{Inf} are shown to correlated with distinct transcriptomic signatures.

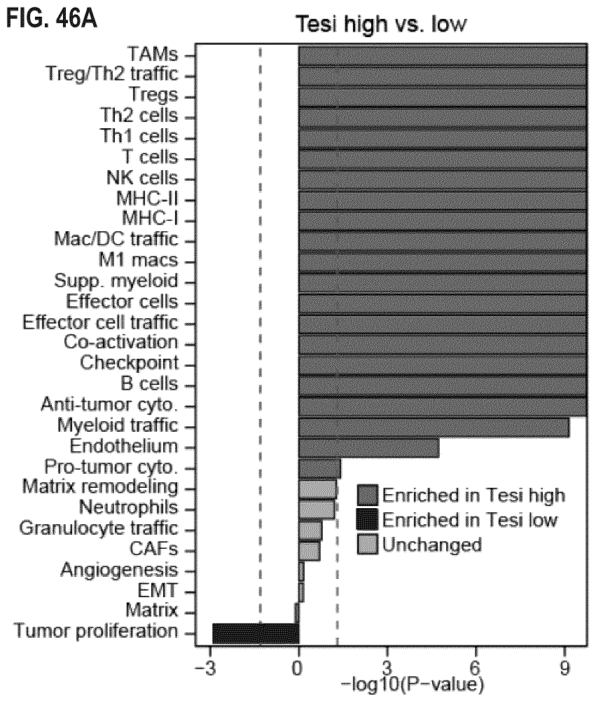

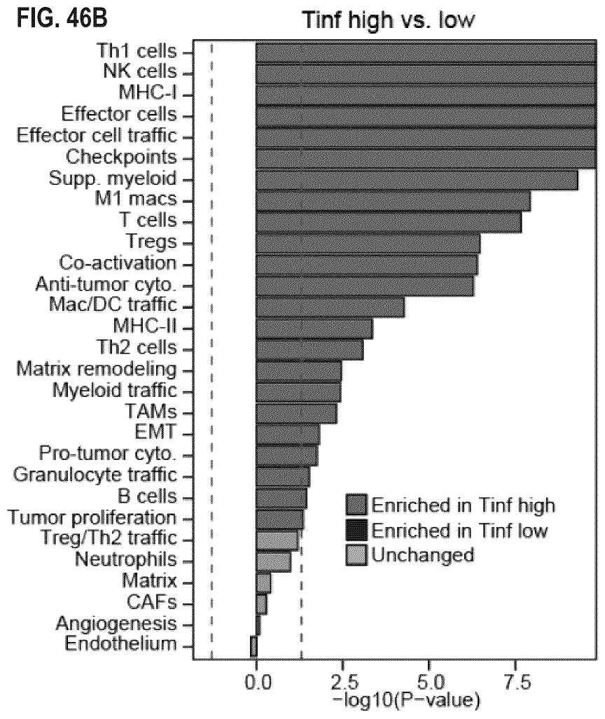

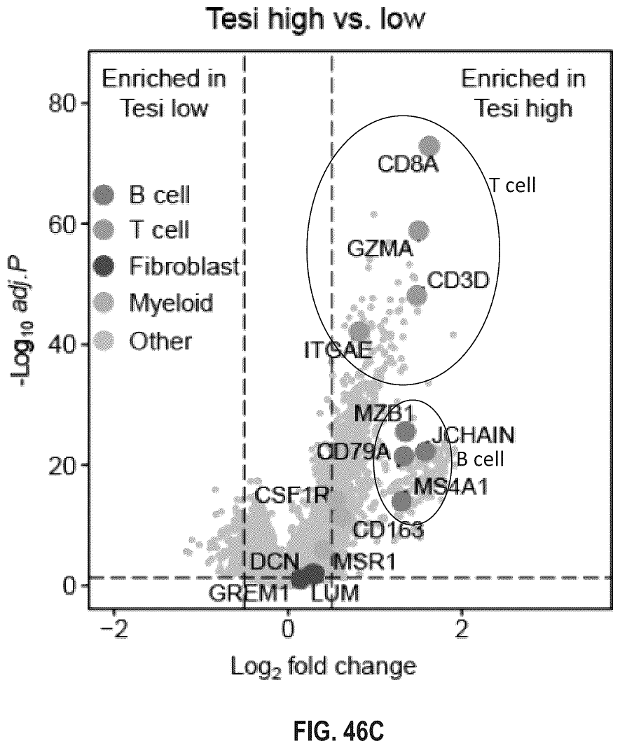

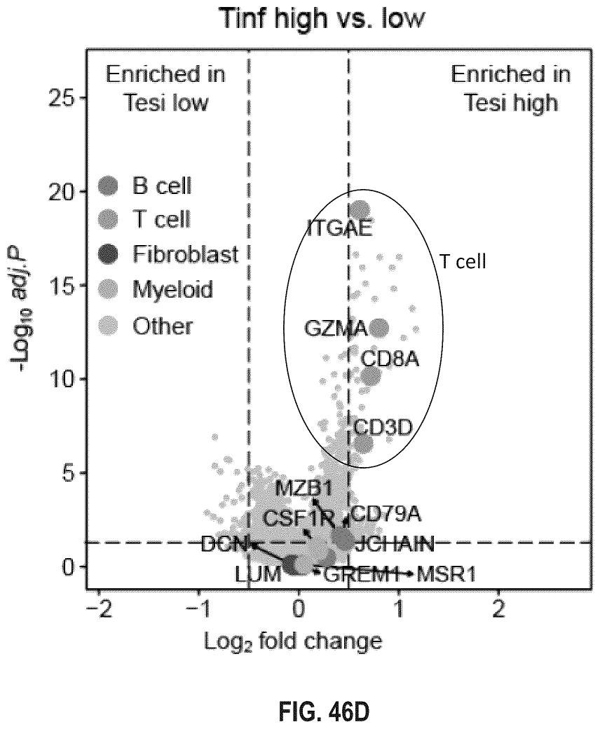

FIGS. 46A-B illustrate RNA-seq gene set analyses comparing T_{ESI} high vs low (FIG. 46A) or T_{inf} high vs, low (FIG. 46B), applying gene signatures corresponding to previously identified cancer and tumor microenvironment cell types and pathway signatures. Dotted lines indicate FDR p-value=0.05 while the sign on the x-axis denotes the direction where positive values indicate enrichment in the high subgroup while negative values indicate enrichment in the low subgroup. Bars extending beyond the axis limits indicate p-values of 0 (log(P-val) of infinity). Colors denote FDR p-value<0.05 matching the color of the specific subset where the gene signature is enriched, grey indicates enrichment indicates FDR p≥0.05. FIGS. 46C-D are RNA-seq volcano plots depicting differentially expressed genes comparing T_{ESI} high vs low (FIG. 46C) and T_{inf} high vs low (FIG. 46D). Highlighted are genes related to the indicated cell types.

Both T_{ESI} and T_{inf} displayed enrichment for T cell-related gene signatures (FIGS. 46A-B) and individual genes (FIGS. 46C-D). Broadly, T_{ESI} appears to strongly enrich signatures related to total immune cell infiltration. For example, various lymphocyte and myeloid genes and signatures were strongly enriched in the T_{ESI}-high subgroup. The T_{inf}-high subgroup displayed a more modest, though still significant, enrichment of pan-immune signatures. In analysis of individual genes, T_{ESI}-high tumors were enriched for both B and T cell related genes, while T_{Inf}-high tumors were specifically enriched for T cell-related genes. For both digital pathology metrics, the greatest enrichment was seen in genes related to CD8 T cells. Overall, this data suggests that both the T_{ESI} and T_{inf}-high groups were enriched for immune-infiltrated NSCLC tumors with a specific enrichment for tumors with high expression of CD8 T cell-related genes.

### Experiment 3: Digital Pathology Features as Biomarkers of Atezolizumab Benefit

Given the mechanism of action of atezolizumab, in which the tumor-killing ability of existing immune cells is unblocked, both T_{ESI} and T_{inf} can be useful predictors of patient survival after treatment with atezolizumab, but not with docetaxel. Greater immune cell density and infiltration into the tumor, as captured by T_{ESI} and T_{inf} would then be associated with better tumor killing and therefore better survival. Since the mechanism of action of docetaxel does not include spurring tumor killing by immune cells, the benefit of enhanced immune presence in the tumor would be lower.

A Cox proportional hazards model is fitted using T_{ESI} and T_{inf} to predict OS in the n=95 atezolizumab-treated patients of the training set. Collection type was included as a stratum in the model, since it was observed that training set patients whose tissue specimens came from a biopsy had worse OS (HR=1.31) than those who underwent resection or excision. This training produced the digital assessment of cytotoxic T-cell infiltration (DACTI) by fitting coefficients β1 and β2 in:

$$DACTI = {β}_{1} {T}_{ESI} + {β}_{2} {T}_{inf}$$

This model was then applied to the entire dataset to calculate each patient's DACTI. The entire training set was used to identify the DACTI threshold which maximized the difference in median survival time between the atezolizumab and docetaxel arms in patients above the threshold. This threshold was then used to categorize validation set patients as DACTI-low or DACTI-high.

This process for selecting an optimal threshold was repeated for each of the CD8 density measures, T_{dens(overal)}, T_{dens(epi)}, T_{dens(stroma)}.

Several statistical analyses were performed. First, the system measured the predictive ability of DACTI as a categorical low/high marker using the OS hazard ratio (HR) between atezolizumab-treated and docetaxel-treated patients in the DACTI-low and DACTI-high groups of the validation set. A biomarker associated with atezolizumab benefit would produce a difference in OS time between atezolizumab-treated patients with low and high DACTI values, without a difference between groups in the docetaxel arm.

Secondly, the system used Harrell's concordance index (c-index) to evaluate DACTI as a continuous marker in the validation set. The c-index measures association of a continuous score with left-censored survival data and ranges from 0 to 1 with a value of 0.5 equivalent to random guessing and a value of 1 reflecting perfect sorting of survival times in descending order. A marker associated with atezolizumab benefit would have a c-index very close to 0.5 in the docetaxel arm and a 95% confidence interval not including 0.5 in the atezolizumab arm. Calculations of c-indexes, HRs, and 95% confidence intervals, were performed using the Cox proportional hazards method of the lifelines Python package, version 0.27.4 (28).

In addition to testing the predictive power of DACTI in the validation set overall, it was also tested separately in the PD-L1-negative (SP142 TC=0 and IC=0) and PDL1-positive subpopulations, as well as in squamous and non-squamous subpopulations. Analysis of these subgroups was prespecified due to the known difference in outcomes between these groups with atezolizumab treatment. Data for a subset of validation set patients (n=401) who had PD-L1 TC scoring from another PD-L1 clone, 22C3, is included in the supplemental material.

Lastly, the interaction between DACTI-low/high category and atezolizumab treatment in predicting OS was tested in the validation set. A Cox regression model was fit on the validation set using DACTI category, treatment arm, and the interaction of the two. A p-value of less than 0.05 for the interaction term would indicate that DACTI category was associated with OS specifically in atezolizumab-treated patients, that is, that DACTI was associated with atezolizumab benefit.

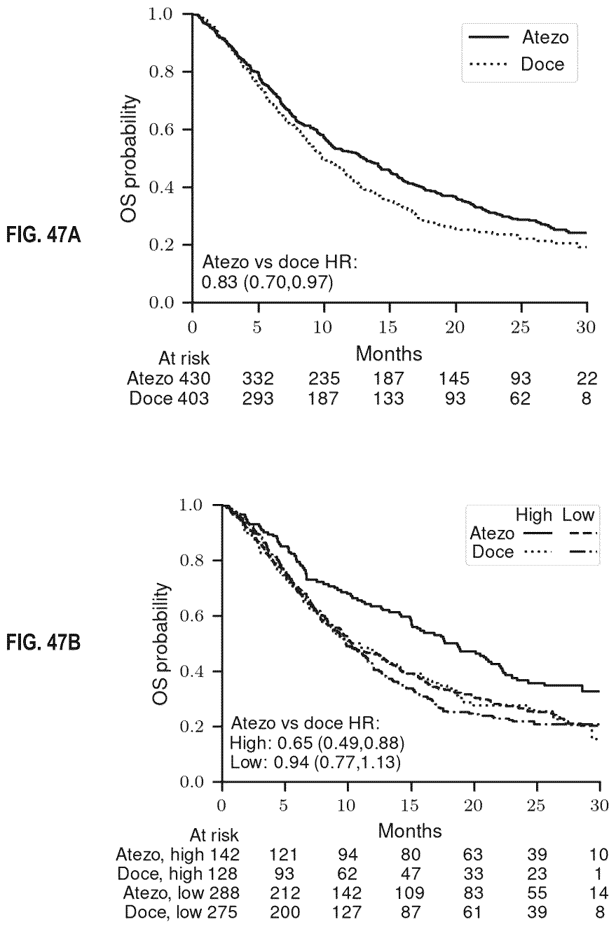

FIGS. 47A-B illustrate overall survival for patients in the validation set stratified by treatment arm (FIG. 47A) and both treatment arm and DACTI category (FIG. 47B). DACTI was positively correlated with OS in atezolizumab-treated patients, but not in docetaxel-treated patients. Among the 270 DACTI-high validation set patients, the atezolizumab arm had significantly longer survival than docetaxel arm (HR=0.65, 95% CI: 0.49-0.88). There was not a significant difference in OS between the arms in the 563 DACTI-low patients (HR=0.94, 95% CI: 0.77-1.13). As a continuous marker, increasing DACTI was significantly associated with longer survival in the atezolizumab arm (c-index=0.56, 95% CI: 0.53-0.59) but not in the docetaxel arm (c-index=0.52, 95% CI: 0.49-0.55).

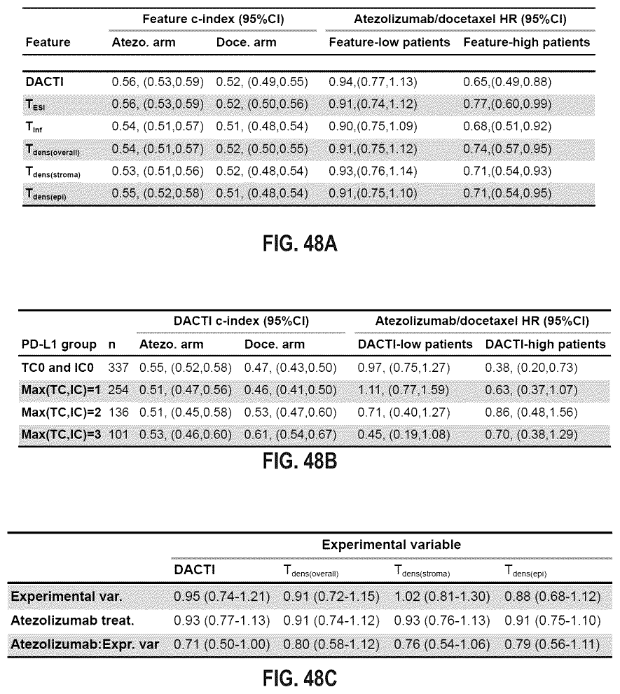

FIG. 48A illustrates predictive performance of DACTI, DACTI's individual component features and T_{ESI}, and T_{Inf},T as well as cell density features T_{dens(overal)}, T_{dens}(epi), T_{dens(stroma)}. DACTI has greater predictive power than either of its component features or any T cell density feature.

FIG. 48B illustrates performance of DACTI in subcohorts of the validation set. Sets are stratified by PD-L1 expression according to the SP142 assay. Shown are the c-index (95% CI) values of DACTI as a continuous measure in the atezolizumab and docetaxel arms, as well as the HRs (95% CI) between the arms in the DACTI-low and DACTI-high groups. DACTI was associated with longer OS on atezolizumab in the PD-L1-negative (TC0 and IC0) subcohort.

FIG. 48C illustrates HRs (95% CI) when using DACTI and three measures of CD8+ cell density as the experimental variable in Cox regression models fitted for OS on the validation set. Each variable was tested in a model containing the experimental variable, atezolizumab treatment, and an interaction term between the experimental variable and atezolizumab treatment. All variables were dichotomized at the optimal threshold identified on the training set. Only DACTI had a significant interaction with atezolizumab treatment.

When patients were stratified by PD-L1 expression by the SP142 assay, the association of DACTI with atezolizumab benefit was seen only in the PD-L1-negative patients. The HR (95% CI) of atezolizumab vs, docetaxel in the DACTI-high group was 0.45 (0.26-0.78) in the 379 IC0 patients and 0.47 (0.32-0.71) in the 591 TC0 patients, with no evidence of difference in OS between the arms in the DACTI-low groups.

DACTI-high status had significant interaction with atezolizumab treatment (HR=0.71 95% CI: 0.50-1.00, p=0.049) in a Cox regression model on the validation set (see Table 3). Neither DACTI category nor atezolizumab treatment alone were associated with OS in this model (p>0.05), suggesting that the majority of atezolizumab benefit was seen in the DACTI-high patients. None of the CD8 density measures had an interaction with atezolizumab treatment in Cox regression models with those terms.

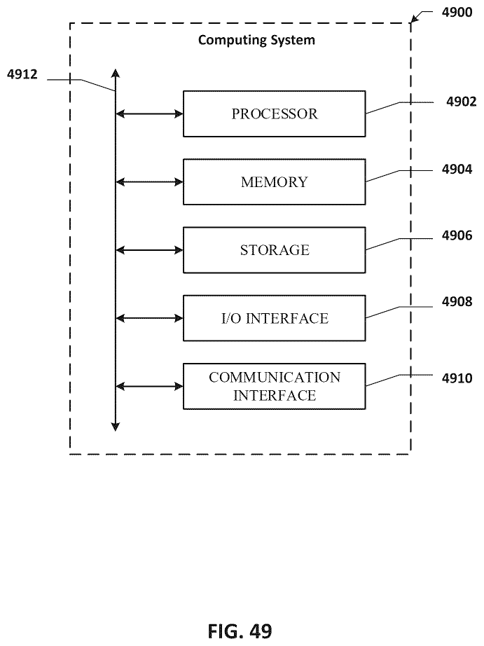

FIG. 49 illustrates an example computer system 4900. In particular embodiments, one or more computer systems 4900 perform one or more steps of one or more methods described or illustrated herein. In particular embodiments, one or more computer systems 4900 provide functionality described or illustrated herein. In particular embodiments, software running on one or more computer systems 4900 performs one or more steps of one or more methods described or illustrated herein or provides functionality described or illustrated herein. Particular embodiments include one or more portions of one or more computer systems 4900. Herein, reference to a computer system may encompass a computing device, and vice versa, where appropriate. Moreover, reference to a computer system may encompass one or more computer systems, where appropriate.

This disclosure contemplates any suitable number of computer systems 4900. This disclosure contemplates computer system 4900 taking any suitable physical form. As example and not by way of limitation, computer system 4900 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 4900 may include one or more computer systems 4900; be unitary or distributed; span multiple locations; span multiple machines; span multiple data centers; or reside in a cloud, which may include one or more cloud components in one or more networks. Where appropriate, one or more computer systems 4900 may perform without substantial spatial or temporal limitation one or more steps of one or more methods described or illustrated herein. As an example, and not by way of limitation, one or more computer systems 4900 may perform in real time or in batch mode one or more steps of one or more methods described or illustrated herein. One or more computer systems 4900 may perform at different times or at different locations one or more steps of one or more methods described or illustrated herein, where appropriate.

In particular embodiments, computer system 4900 includes a processor 4902, memory 4904, storage 4906, an input/output (I/O) interface 4908, a communication interface 4910, and a bus 4912. Although this disclosure describes and illustrates a particular computer system having a particular number of particular components in a particular arrangement, this disclosure contemplates any suitable computer system having any suitable number of any suitable components in any suitable arrangement.

In particular embodiments, processor 4902 includes hardware for executing instructions, such as those making up a computer program. As an example, and not by way of limitation, to execute instructions, processor 4902 may retrieve (or fetch) the instructions from an internal register, an internal cache, memory 4904, or storage 4906; decode and execute them; and then write one or more results to an internal register, an internal cache, memory 4904, or storage 4906. In particular embodiments, processor 4902 may include one or more internal caches for data, instructions, or addresses. This disclosure contemplates processor 4902 including any suitable number of any suitable internal caches, where appropriate. As an example, and not by way of limitation, processor 4902 may include one or more instruction caches, one or more data caches, and one or more translation lookaside buffers (TLBs). Instructions in the instruction caches may be copies of instructions in memory 4904 or storage 4906, and the instruction caches may speed up retrieval of those instructions by processor 4902. Data in the data caches may be copies of data in memory 4904 or storage 4906 for instructions executing at processor 4902 to operate on; the results of previous instructions executed at processor 4902 for access by subsequent instructions executing at processor 4902 or for writing to memory 4904 or storage 4906; or other suitable data. The data caches may speed up read or write operations by processor 4902. The TLBs may speed up virtual-address translation for processor 4902. In particular embodiments, processor 4902 may include one or more internal registers for data, instructions, or addresses. This disclosure contemplates processor 4902 including any suitable number of any suitable internal registers, where appropriate. Where appropriate, processor 4902 may include one or more arithmetic logic units (ALUs); be a multi-core processor; or include one or more processors 4902. Although this disclosure describes and illustrates a particular processor, this disclosure contemplates any suitable processor.

In particular embodiments, memory 4904 includes main memory for storing instructions for processor 4902 to execute or data for processor 4902 to operate on. As an example, and not by way of limitation, computer system 4900 may load instructions from storage 4906 or another source (such as, for example, another computer system 4900) to memory 4904. Processor 4902 may then load the instructions from memory 4904 to an internal register or internal cache. To execute the instructions, processor 4902 may retrieve the instructions from the internal register or internal cache and decode them. During or after execution of the instructions, processor 4902 may write one or more results (which may be intermediate or final results) to the internal register or internal cache. Processor 4902 may then write one or more of those results to memory 4904. In particular embodiments, processor 4902 executes only instructions in one or more internal registers or internal caches or in memory 4904 (as opposed to storage 4906 or elsewhere) and operates only on data in one or more internal registers or internal caches or in memory 4904 (as opposed to storage 4906 or elsewhere). One or more memory buses (which may each include an address bus and a data bus) may couple processor 4902 to memory 4904. Bus 4912 may include one or more memory buses, as described below. In particular embodiments, one or more memory management units (MMUs) reside between processor 4902 and memory 4904 and facilitate accesses to memory 4904 requested by processor 4902. In particular embodiments, memory 4904 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 4904 may include one or more memories 3404, where appropriate. Although this disclosure describes and illustrates particular memory, this disclosure contemplates any suitable memory.

In particular embodiments, storage 4906 includes mass storage for data or instructions. As an example, and not by way of limitation, storage 4906 may include a hard disk drive (HDD), a floppy disk drive, flash memory, an optical disc, a magneto-optical disc, magnetic tape, or a Universal Serial Bus (USB) drive or a combination of two or more of these. Storage 4906 may include removable or non-removable (or fixed) media, where appropriate. Storage 4906 may be internal or external to computer system 4900, where appropriate. In particular embodiments, storage 4906 is non-volatile, solid-state memory. In particular embodiments, storage 4906 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 4906 taking any suitable physical form. Storage 4906 may include one or more storage control units facilitating communication between processor 4902 and storage 4906, where appropriate. Where appropriate, storage 4906 may include one or more storages 3406. Although this disclosure describes and illustrates particular storage, this disclosure contemplates any suitable storage.

In particular embodiments, I/O interface 4908 includes hardware, software, or both, providing one or more interfaces for communication between computer system 4900 and one or more I/O devices. Computer system 4900 may include one or more of these I/O devices, where appropriate. One or more of these I/O devices may enable communication between a person and computer system 4900. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 4908 for them. Where appropriate, I/O interface 4908 may include one or more device or software drivers enabling processor 4902 to drive one or more of these I/O devices. I/O interface 4908 may include one or more I/O interfaces 4908, where appropriate. Although this disclosure describes and illustrates a particular I/O interface, this disclosure contemplates any suitable I/O interface.

In particular embodiments, communication interface 4910) includes hardware, software, or both providing one or more interfaces for communication (such as, for example, packet-based communication) between computer system 4900 and one or more other computer systems 4900 or one or more networks. As an example, and not by way of limitation, communication interface 4910 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 4910 for it. As an example, and not by way of limitation, computer system 4900 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 4900 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 4900 may include any suitable communication interface 4910 for any of these networks, where appropriate. Communication interface 4910 may include one or more communication interfaces 4910, where appropriate. Although this disclosure describes and illustrates a particular communication interface, this disclosure contemplates any suitable communication interface.

In particular embodiments, bus 4912 includes hardware, software, or both coupling components of computer system 4900 to each other. As an example and not by way of limitation, bus 4912 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 4912 may include one or more buses 4912, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICS (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

### EXAMPLE EMBODIMENTS

Embodiments disclosed herein may include:
1. A method for determining an immunophenotype of a tumor using a computing system, the method comprising:
receiving an image of a tumor;
dividing the image into a plurality of tiles each depicting at least one of tumor epithelium or tumor stroma;
for each of the plurality of tiles:
   calculating an epithelium-immune cell density of the tile based on a number of immune cells identified in the tumor epithelium or calculating a stroma-immune cell density of the tile based on a number of immune cells identified in the tumor stroma; and
   determining based on the stroma-immune cell density and/or the epithelium-immune cell density, an inflammation type of the tile as being a first inflammation type or a second inflammation type; and
determining a tumor immunophenotype for the image based on the inflammation type of the plurality of tiles.
2. The method of embodiment 1, wherein the tumor immunophenotype comprises:
desert based on a number of tiles of the plurality of tiles of the first inflammation type being less than a first threshold and a number of tiles of the plurality of tiles of the second inflammation type being less than a second threshold;
excluded based on the number of tiles of the plurality of tiles of the first inflammation type being greater than or equal to the first threshold and the number of tiles of the plurality of tiles of the second inflammation type being less than the second threshold; or
inflamed based on the number of tiles of the plurality of tiles of the first inflammation type being greater than or equal to the first threshold and the number of tiles of the plurality of tiles of the second inflammation type being greater than or equal to the second threshold.
3. The method of any one of embodiments 1-2, wherein the inflammation type comprises:
the first inflammation type based on (i) a first stroma criterion for the stroma-immune cell density being met and (ii) a second stroma criterion for the epithelium-immune cell density being met; or
the second inflammation type based on (iii) a first epithelium criterion for the stroma-immune cell density being met and (iv) a second epithelium criterion for the epithelium-immune cell density being met.
4. The method of embodiment 3, wherein;
the first stroma criterion for the stroma-immune cell density being met comprises the stroma-immune cell density being greater than or equal to a stroma-immune cell density threshold;
the second stroma criterion for the epithelium-immune cell density being met comprises the epithelium-immune cell density being less than or equal to an epithelium-immune cell density threshold;
the first epithelium criterion for the stroma-immune cell density being met comprises the stroma-immune cell density being less than the stroma-immune cell density threshold; and
the second epithelium criterion for the epithelium-immune cell density being met comprises the epithelium-immune cell density being less than the epithelium-immune cell density threshold.
5. The method of embodiment 4, wherein the stroma-immune cell density threshold and the epithelium-immune cell density threshold are based on a number of immune cells at an epithelium-stroma interface.
6. The method of embodiment 4, wherein the stroma-immune cell density threshold and the epithelium-immune cell density threshold are based on a number of immune cells at an epithelium-stroma interface divided by a total number of tiles of the plurality of tiles.
7. The method of embodiment 4, wherein the stroma-immune cell density threshold is based on a distribution of immune cells in the tumor stroma and the epithelium-immune cell density threshold is based on a distribution of immune cells in the tumor epithelium, wherein the distribution of immune cells in the tumor stroma and the distribution of immune cells in the tumor epithelium is based on a plurality of distance measurements.
8. The method of embodiment 7, further comprising:
determining the plurality of distance measurements, comprising:
   performing a color deconvolution to generate a color channel highlighting cell nuclei;
   identifying based on the color channel, a plurality of immune cell nuclei; and
   calculating the plurality of distance measurements each representing a distance from one of the plurality of immune cell nuclei to an epithelium-stroma interface.
9. The method of any one of embodiments 1-8, further comprising:
performing a color deconvolution to generate a plurality of color channels from the image, the plurality of color channels including at least a first color channel and a second color channel, wherein the first color channel highlights immune cells and the second color channel distinguishes the tumor epithelium from the tumor stroma.
10. The method of any one of embodiments 1-9, further comprising:
determining a correction factor based on a number of immune cells at an epithelium-stroma interface; and
modifying based on the correction factor, at least one of the calculated stroma-immune cell density or the calculated epithelium-immune cell density of at least some of the plurality of tiles.
11. The method of any one of embodiments 1-10, wherein at least some of the plurality of tiles are overlapping.
12. The method of any one of embodiments 1-11, wherein at least one of the plurality of tiles contains a unique portion of the image.
13. The method of any one of embodiments 1-12, wherein at least one of the plurality of tiles comprises a random or pseudo-random subset of the plurality of tiles of the image.
14. The method of any one of embodiments 1-13, wherein the image comprises the tumor stained with one or more stains, wherein the one or more stains comprise at least one of:
a pan-cytokeratin (panCK) stain used for highlighting the tumor epithelium;
a cluster of differentiation 8 (CD8) stain used for highlighting immune cells; or
a hematoxylin stain used for highlighting one or more of; cell nuclei, an extracellular matrix, or cell cytoplasm.
15. The method of any one of embodiments 1-14, further comprising:
identifying a boundary of the tumor in a digital pathology image; and
extracting based on the boundary, the image of the tumor from the digital pathology image.
16. The method of embodiment 15, wherein identifying the boundary comprises:
providing the digital pathology image to a computer vision model trained to detect the boundary of the tumor; and
receiving an indication of the boundary from the computer vision model.
17. The method of any one of embodiments 1-16, further comprising:
selecting based on the tumor immunophenotype, an immunotherapy for a patient.
18. The method of any one of embodiments 1-17, further comprising:
identifying artifacts in the image; and
removing the artifacts from the image.
19. A system for determining an immunophenotype of a tumor, comprising:
a computing system comprising
   one or more non-transitory computer-readable storage media storing computer program instructions; and
   one or more processors coupled to the one or more non-transitory computer-readable storage media, the one or more processors being configured to execute the computer program instructions to:
      receive an image of a tumor region;
      divide the image into a plurality of tiles each depicting at least one of tumor epithelium or tumor stroma;
      for each of the plurality of tiles:
         calculate an epithelium-immune cell density of the tile based on a number of immune cells identified in the tumor epithelium or calculate a stroma-immune cell density of the tile based on a number of immune cells identified in the tumor stroma; and
         determine, based on the stroma-immune cell density and/or the epithelium-immune cell density, an inflammation type of the tile as being a first inflammation type or a second inflammation type; and
      determine a tumor immunophenotype for the image based on the inflammation type of the plurality of tiles.
20. A non-transitory computer-readable medium comprising computer program instructions that, when executed by one or more processors of a computing system, effectuate operations comprising:
receiving an image of a tumor region;
dividing the image into a plurality of tiles each depicting at least one of tumor epithelium or tumor stroma;
for each of the plurality of tiles:
   calculating an epithelium-immune cell density of the tile based on a number of immune cells identified in the tumor epithelium or calculating a stroma-immune cell density of the tile based on a number of immune cells identified in the tumor stroma; and
   determining, based on the stroma-immune cell density and/or the epithelium-immune cell density, an inflammation type of the tile as being a first inflammation type or a second inflammation type; and
determine a tumor immunophenotype for the image based on the inflammation type of the plurality of tiles.
21. A method for determining an immunophenotype of a tumor using a computing system, the method comprising:
receiving an image of a tumor;
identifying, based on the image, an epithelium-stroma interface separating tumor epithelium and tumor stroma using one or more machine learning models;
determining an epithelium-stroma interface immune cell density based on the image, wherein the epithelium-stroma immune cell density represents a number of immune cells within a threshold distance of the epithelium-stroma interface;
determining an immune cell infiltration probability of immune cells in the tumor stroma infiltrating the tumor epithelium; and
determining a tumor immunophenotype of the image based on the epithelium-stroma interface immune cell density and the immune cell infiltration probability.
22. The method of embodiment 21, wherein identifying the epithelium-stroma interface comprises:
separating the image into a plurality of color channels, wherein a first color channel of the plurality of color channels highlighting the immune cells and a second color channel distinguishing the tumor epithelium from the tumor stroma.
23. The method of embodiment 22, wherein identifying the epithelium-stroma interface comprises:
identifying a boundary of the tumor in a digital pathology image the second color channel distinguishing the tumor epithelium and the tumor stroma, wherein the boundary comprises the epithelium-stroma interface; and
extracting the image of the tumor from the digital pathology image based on the boundary.
24. The method of embodiment 23, wherein the one or more machine learning models comprise a computer vision model, the method further comprises:
providing the image to the computer vision model trained to identify pixels highlighting the tumor epithelium and pixels highlighting the tumor stroma; and
receiving an indication of the epithelium-stroma interface from the computer vision model.
25. The method of any one of embodiments 21-24, wherein the image of the tumor is stained with one or more stains, wherein the one or more stains comprise at least one of:
a pan-cytokeratin (panCK) stain used for highlighting the tumor epithelium;
a cluster of differentiation 8 (CD8) stain used for highlighting immune cells; or
a hematoxylin stain used for highlighting one or more of; cell nuclei, an extracellular matrix, or cell cytoplasm.
26. The method of any one of embodiments 21-25, wherein determining the epithelium-stroma interface immune cell density comprises:
separating the image into a plurality of color channels, wherein the plurality of color channels comprises a first color channel of the plurality of color channels highlighting the immune cells and at least a second color channel of the plurality of color channels distinguishing the tumor epithelium from the tumor stroma;
determining, based on the plurality of color channels, the number of immune cells within the threshold distance of the epithelium-stroma interface.
27. The method of embodiment 26, wherein the threshold distance of the epithelium-stroma interface defines;
a first distance from the epithelium-stroma interface into the tumor stroma; and
a second distance from the epithelium-stroma interface into the tumor epithelium, and wherein the number of immune cells within the threshold distance comprises immune cells located within the first distance from the epithelium-stroma interface and immune cells located within the second distance from the epithelium-stroma interface.
28. The method of embodiment 27, wherein at least one of the first distance or the second distance comprises 1 micron or less from the epithelium-stroma interface, 2 microns or less from the epithelium-stroma interface, 5 microns or less from the epithelium-stroma interface, 10 microns or less of the epithelium-stroma interface, or 20 microns or less from the epithelium-stroma interface.
29. The method of any one of embodiments 21-28, wherein determining the immune cell infiltration probability comprises:
computing a ratio of a number of immune cells depicted in the image that are located within the tumor stroma and a number of immune cells depicted in the image that are located within the tumor epithelium, wherein the immune cell infiltration probability is based on the ratio.
30. The method of any one of embodiments 21-29, further comprising:
accessing tumor immunophenotype classification data representing a tumor immunophenotype classification of each of a plurality of images of tumors based on an epithelium-stroma interface immune cell density and an immune cell infiltration probability of each of the plurality of images, wherein determining the tumor immunophenotype of the image comprises:
   classifying the image of the tumor into one of a set of tumor immunophenotypes based on the tumor immunophenotype classification data, the epithelium-stroma interface immune cell density of the image, and the immune cell infiltration probability of the image.
31. The method of embodiment 30, wherein a trained classifier is used for classifying the image.
32. The method of any one of embodiments 30-31, further comprising:
for each of the plurality of images;
   identifying an epithelium-stroma interface;
   determining an epithelium-stroma interface immune cell density;
   determining an immune cell infiltration probability; and
   determining a tumor immunophenotype of the respective image based on the epithelium-stroma interface immune cell density of the respective image and the immune cell infiltration probability of the respective image.
33. The method of embodiment 32, wherein the epithelium-stroma interface immune cell density of each of the plurality of images is determined using the one or more machine learning models.
34. The method of any one of embodiments 32-33, wherein the set of tumor immunophenotypes comprises a first tumor immunophenotype and a second tumor immunophenotype.
35. The method of embodiment 34, further comprising:
computing a median epithelium-stroma interface immune cell density based the epithelium-stroma interface immune cell density of each of the plurality of images, wherein the image of the tumor is classified into one of the set of tumor immunophenotypes based on the median epithelium-stroma interface immune cell density and the immune cell infiltration probability.
36. The method of embodiment 35, wherein the image is classified into one of the set of tumor immunophenotypes based on the median epithelium-stroma interface immune cell density.
37. The method of any one of embodiments 35-36, wherein the median epithelium-stroma interface immune cell density is less than 40 immune cells/mm², less than 60 immune cells/mm², or less than 100 immune cells/mm².
38. The method of any one of embodiments 35-37, wherein the median epithelium-stroma interface immune cell density is approximately 56 immune cells/mm².
39. The method of any one of embodiments 35-38, wherein;
the first tumor immunophenotype is based on the epithelium-stroma interface immune cell density being less than the median epithelium-stroma interface immune cell density; and
the second tumor immunophenotype is based on the epithelium-stroma interface immune cell density being greater than or equal to the median epithelium-stroma interface immune cell density.
40. The method of any one of embodiments 32-39, wherein the set of tumor immunophenotypes comprises:
desert based on the epithelium-stroma interface immune cell density and the immune cell infiltration probability satisfying desert immunophenotype classification criteria;
excluded based on the epithelium-stroma interface immune cell density and the immune cell infiltration probability satisfying excluded immunophenotype classification criteria; or
inflamed based on the epithelium-stroma interface immune cell density and the immune cell infiltration probability satisfying inflamed immunophenotype classification criteria.
41. The method of embodiment 40, wherein the desert immunophenotype classification criteria being satisfied comprises:
the epithelium-stroma interface immune cell density being within a first threshold range of epithelium-stroma interface immune cell densities; and
the immune cell infiltration probability being within a first threshold range of immune cell infiltration probabilities.
42. The method of any one of embodiments 40-41, wherein the excluded immunophenotype classification criteria being satisfied comprises:
the epithelium-stroma interface immune cell density being within a second threshold range of epithelium-stroma interface immune cell densities; and
the immune cell infiltration probability being within a second threshold range of immune cell infiltration probabilities.
43. The method of any one of embodiments 40-42, wherein the inflamed immunophenotype classification criteria being satisfied comprises:
the epithelium-stroma interface immune cell density being within a third threshold range of epithelium-stroma interface immune cell densities; and
the immune cell infiltration probability being within a third threshold range of immune cell infiltration probabilities.
44. The method of any one of embodiments 21-43, further comprising:
selecting an immunotherapy for a patient based on the tumor immunophenotype.
45. A system for determining an immunophenotype of a tumor, comprising:
a computing system comprising
   one or more non-transitory computer-readable storage media storing computer program instructions; and
   one or more processors coupled to the one or more non-transitory computer-readable storage media, the one or more processors being configured to execute the computer program instructions to:
      receive an image of a tumor;
      identify, based on the image, an epithelium-stroma interface separating tumor epithelium and tumor stroma using one or more machine learning models;
      determine an epithelium-stroma interface immune cell density based on the image, wherein the epithelium-stroma interface immune cell density represents a number of immune cells within a threshold distance of the epithelium-stroma interface;
      determine an immune cell infiltration probability of immune cells in the tumor stroma infiltrating the tumor epithelium; and
      determine a tumor immunophenotype of the image based on the epithelium-stroma interface immune cell density and the immune cell infiltration probability.
46. A non-transitory computer-readable medium comprising computer program instructions that, when executed by one or more processors of a computing system, effectuate operations comprising:
receiving an image of a tumor;
identifying, based on the image, an epithelium-stroma interface separating tumor epithelium and tumor stroma using one or more machine learning models;
determining an epithelium-stroma interface immune cell density based on the image, wherein the epithelium-stroma interface immune cell density represents a number of immune cells within a threshold distance of the epithelium-stroma interface;
determining an immune cell infiltration probability of immune cells in the tumor stroma infiltrating the tumor epithelium; and
determining a tumor immunophenotype of the image based on the epithelium-stroma interface immune cell density and the immune cell infiltration probability.
47. A method for predicting a response to an anti-PD-L1 treatment by a patient, comprising:
receiving an image of a tumor of the patient;
identifying, based on the image, a plurality of immune cells in the image;
identifying, based on the image, an epithelium-stroma interface (ESI) separating tumor epithelium and tumor stroma in the image;
determining an ESI immune cell density based on the image, wherein the epithelium-stroma immune cell density represents a measure of immune cells within a threshold distance of the ESI;
determining immune cell infiltration based on the image, wherein the immune cell infiltration represents a measure of infiltration into the tumor epithelium by immune cells; and
predicting the response to the anti-PD-L1 treatment by inputting the ESI immune cell density and the immune cell infiltration into a model.
48. The method of embodiment 47, wherein the ESI immune cell density comprises a ratio of a number of immune cells within the threshold distance of the ESI and a total number of cells within the threshold distance of the ESI.
49. The method of embodiment 47, wherein the threshold distance is 8 microns.
50. The method of any one of embodiments 47-49, wherein the immune cell infiltration comprises a ratio between a first ratio and a second ratio, wherein the first ratio is between a number of immune cells and a total number of cells within an area of the tumor epithelium, and wherein the second ratio is between a number of immune cells and a total number of cells within an area of the tumor stroma.
51. The method of embodiment 50, wherein the area of the tumor epithelium is between a first distance and a second distance from the ESI in the tumor epithelium.
52. The method of embodiment 51, wherein the area of the tumor stroma is between the first distance and the second distance from the ESI in the tumor stroma.
53. The method of embodiments 50-52, wherein the first distance is 24 microns and the second distance is 8 microns.
54. The method of any one of embodiments 47-53, wherein identifying the plurality of immune cells comprises:
identifying a plurality of cells in the image; and
determining whether each cell of the plurality of cells in the image is an immune cell based on a color channel of the image, the color channel highlighting immune cells in the image.
55. The method of embodiment 54, wherein the color channel highlighting immune cells in the image corresponds to a CD8 stain map of the image.
56. The method of any one of embodiments 47-55, wherein identifying the ESI in the image comprises: identifying a tumor stroma area in the image.
57. The method of embodiment 56, wherein identifying the tumor stroma area in the image comprises: identifying a first group of pixels in the image based on a luminosity threshold and a color channel distinguishing between the tumor stroma and the tumor epithelium.
58. The method of embodiment 57, wherein the color channel distinguishing between the tumor stroma and the tumor epithelium corresponds to a panCK stain map of the image.
59. The method of embodiment 57 or 58, wherein identifying the tumor stroma area in the image further comprises: identifying a second group of pixels in the image based on a plurality of cell nuclei in the tumor stroma according to the color channel distinguishing between the tumor stroma and the tumor epithelium.
60. The method of embodiment 59, wherein the tumor stroma area is a union of the first group of pixels and the second group of pixels.
61. The method of any one of embodiments 47-60, wherein the model comprises a machine-learning model or a statistical model.
62. The method of embodiment 61, wherein the model comprises a fitted Cox proportional hazards model.
63. The method of any one of embodiments 47-62, wherein predicting the response to the anti-PD-L1 treatment comprises:
obtaining a treatment response prediction score from the model; and
comparing the treatment response against a predefined threshold.
64. The method of any one of embodiments 47-63, wherein the anti-PD-L1 treatment comprises: atezolizumab, avelumab, or durvalumab.
65. A system for determining an immunophenotype of a tumor, comprising:
one or more non-transitory computer-readable storage media storing computer program instructions; and
one or more processors coupled to the one or more non-transitory computer-readable storage media, the one or more processors being configured to execute the computer program instructions to:
   receive an image of a tumor of the patient;
   identify, based on the image, a plurality of immune cells in the image;
   identify, based on the image, an epithelium-stroma interface (ESI) separating tumor epithelium and tumor stroma in the image;
   determine an ESI immune cell density based on the image, wherein the epithelium-stroma immune cell density represents a measure of immune cells within a threshold distance of the ESI;
   determine immune cell infiltration based on the image, wherein the immune cell infiltration represents a measure of infiltration into the tumor epithelium by immune cells; and
   predict the response to the anti-PD-L1 treatment by inputting the ESI immune cell density and the immune cell infiltration into a model.
66. A non-transitory computer-readable medium comprising computer program instructions that, when executed by one or more processors of a computing system, effectuate operations comprising:
receiving an image of a tumor of the patient;
identifying, based on the image, a plurality of immune cells in the image;
identifying, based on the image, an epithelium-stroma interface (ESI) separating tumor epithelium and tumor stroma in the image;
determining an ESI immune cell density based on the image, wherein the epithelium-stroma immune cell density represents a measure of immune cells within a threshold distance of the ESI;
determining immune cell infiltration based on the image, wherein the immune cell infiltration represents a measure of infiltration into the tumor epithelium by immune cells;
and
predicting the response to the anti-PD-L1 treatment by inputting the ESI immune cell density and the immune cell infiltration into a model.

What we claim is:

1. A method for determining an immunophenotype of a tumor using a computing system, the method comprising:
receiving an image of a tumor:
dividing the image into a plurality of tiles each depicting at least one of tumor epithelium or tumor stroma:
for each of the plurality of tiles:
calculating an epithelium-immune cell density of the tile based on a number of immune cells identified in the tumor epithelium or calculating a stroma-immune cell density of the tile based on a number of immune cells identified in the tumor stroma; and
determining based on the stroma-immune cell density and/or the epithelium-immune cell density, an inflammation type of the tile as being a first inflammation type or a second inflammation type; and
determining a tumor immunophenotype for the image based on the inflammation type of the plurality of tiles.

2. The method of claim 1, wherein the tumor immunophenotype comprises:
desert based on a number of tiles of the plurality of tiles of the first inflammation type being less than a first threshold and a number of tiles of the plurality of tiles of the second inflammation type being less than a second threshold:
excluded based on the number of tiles of the plurality of tiles of the first inflammation type being greater than or equal to the first threshold and the number of tiles of the plurality of tiles of the second inflammation type being less than the second threshold; or
inflamed based on the number of tiles of the plurality of tiles of the first inflammation type being greater than or equal to the first threshold and the number of tiles of the plurality of tiles of the second inflammation type being greater than or equal to the second threshold.

3. The method of claim 1, wherein the inflammation type comprises:
the first inflammation type based on (i) a first stroma criterion for the stroma-immune cell density being met and (ii) a second stroma criterion for the epithelium-immune cell density being met; or
the second inflammation type based on (iii) a first epithelium criterion for the stroma-immune cell density being met and (iv) a second epithelium criterion for the epithelium-immune cell density being met.

4. The method of claim 3, wherein:
the first stroma criterion for the stroma-immune cell density being met comprises the stroma-immune cell density being greater than or equal to a stroma-immune cell density threshold;
the second stroma criterion for the epithelium-immune cell density being met comprises the epithelium-immune cell density being less than or equal to an epithelium-immune cell density threshold;
the first epithelium criterion for the stroma-immune cell density being met comprises the stroma-immune cell density being less than the stroma-immune cell density threshold; and
the second epithelium criterion for the epithelium-immune cell density being met comprises the epithelium-immune cell density being less than the epithelium-immune cell density threshold.

5. The method of claim 4, wherein the stroma-immune cell density threshold and the epithelium-immune cell density threshold are based on a number of immune cells at an epithelium-stroma interface.

6. The method of claim 4, wherein the stroma-immune cell density threshold and the epithelium-immune cell density threshold are based on a number of immune cells at an epithelium-stroma interface divided by a total number of tiles of the plurality of tiles.

7. The method of claim 4, wherein the stroma-immune cell density threshold is based on a distribution of immune cells in the tumor stroma and the epithelium-immune cell density threshold is based on a distribution of immune cells in the tumor epithelium, wherein the distribution of immune cells in the tumor stroma and the distribution of immune cells in the tumor epithelium is based on a plurality of distance measurements.

8. The method of claim 7, further comprising:
determining the plurality of distance measurements, comprising:
performing a color deconvolution to generate a color channel highlighting cell nuclei;
identifying based on the color channel, a plurality of immune cell nuclei; and
calculating the plurality of distance measurements each representing a distance from one of the plurality of immune cell nuclei to an epithelium-stroma interface.

9. The method of claim 1, further comprising:
performing a color deconvolution to generate a plurality of color channels from the image, the plurality of color channels including at least a first color channel and a second color channel, wherein the first color channel highlights immune cells and the second color channel distinguishes the tumor epithelium from the tumor stroma.

10. The method of claim 1, further comprising:
determining a correction factor based on a number of immune cells at an epithelium-stroma interface; and
modifying based on the correction factor, at least one of the calculated stroma-immune cell density or the calculated epithelium-immune cell density of at least some of the plurality of tiles.

11. The method of claim 1, wherein at least some of the plurality of tiles are overlapping.

12. The method of claim 1, wherein at least one of the plurality of tiles contains a unique portion of the image.

13. The method of claim 1, wherein at least one of the plurality of tiles comprises a random or pseudo-random subset of the plurality of tiles of the image.

14. The method of claim 1, wherein the image comprises the tumor stained with one or more stains, wherein the one or more stains comprise at least one of:
a pan-cytokeratin (panCK) stain used for highlighting the tumor epithelium;
a cluster of differentiation 8 (CD8) stain used for highlighting immune cells; or
a hematoxylin stain used for highlighting one or more of: cell nuclei, an extracellular matrix, or cell cytoplasm.

15. The method of claim 1, further comprising:
identifying a boundary of the tumor in a digital pathology image; and
extracting based on the boundary, the image of the tumor from the digital pathology image.

16. The method of claim 15, wherein identifying the boundary comprises:
providing the digital pathology image to a computer vision model trained to detect the boundary of the tumor; and
receiving an indication of the boundary from the computer vision model.

17. The method of claim 1, further comprising:
selecting based on the tumor immunophenotype, an immunotherapy for a patient.

18. The method of claim 1, further comprising:
identifying artifacts in the image; and
removing the artifacts from the image.

19. A system for determining an immunophenotype of a tumor, comprising:
a computing system comprising
one or more non-transitory computer-readable storage media storing computer program instructions; and
one or more processors coupled to the one or more non-transitory computer-readable storage media, the one or more processors being configured to execute the computer program instructions to:
receive an image of a tumor region;
divide the image into a plurality of tiles each depicting at least one of tumor epithelium or tumor stroma;
for each of the plurality of tiles:
calculate an epithelium-immune cell density of the tile based on a number of immune cells identified in the tumor epithelium or calculate a stroma-immune cell density of the tile based on a number of immune cells identified in the tumor stroma; and
determine, based on the stroma-immune cell density and/or the epithelium-immune cell density, an inflammation type of the tile as being a first inflammation type or a second inflammation type; and
determine a tumor immunophenotype for the image based on the inflammation type of the plurality of tiles.

20. A non-transitory computer-readable medium comprising computer program instructions that, when executed by one or more processors of a computing system, effectuate operations comprising:
receiving an image of a tumor region;
dividing the image into a plurality of tiles each depicting at least one of tumor epithelium or tumor stroma;
for each of the plurality of tiles:
calculating an epithelium-immune cell density of the tile based on a number of immune cells identified in the tumor epithelium or calculating a stroma-immune cell density of the tile based on a number of immune cells identified in the tumor stroma; and
determining, based on the stroma-immune cell density and/or the epithelium-immune cell density, an inflammation type of the tile as being a first inflammation type or a second inflammation type; and
determine a tumor immunophenotype for the image based on the inflammation type of the plurality of tiles.
